(19) United States
(12) Patent Application Publication
(10) Pub. No.: US 2024/0417443 A1
(43) Pub. Date: Dec. 19, 2024

(54) IL5RA CELL SURFACE MARKERS

(71) Applicant: Sonoma Biotherapeutics, Inc., South San Francisco, CA (US)

(72) Inventors: James MATTHAEI, Seattle, WA (US); Joshua BEILKE, Sammamish, WA (US); Trevor MILEUR, Seattle, WA (US)

(73) Assignee: Sonoma Biotherapeutics, Inc., South San Francisco, CA (US)

(21) Appl. No.: 18/693,418

(22) PCT Filed: Sep. 22, 2022
(86) PCT No.: PCT/US2022/076862

§ 371 Date: Mar. 19, 2024

Related U.S. Application Data
(60) Provisional application No. 63/247,239, filed on Sep. 22, 2021.

(51) Int. Cl. C07K 14/715; A61K 39/00; C07K 16/18; C12N 5/0783

(52) U.S. Cl. CPC C07K 14/7155; A61K 39/4611; A61K 39/4631; A61K 39/4643; C07K 16/18; C12N 5/0637; C07K 2317/732; C07K 2319/02; C07K 2319/03; C12N 2510/00

(57) ABSTRACT

The present invention provides cellular tags including an extracellular region, a transmembrane region, and an optional intracellular region. The extracellular region comprises an IL5 receptor alpha (IL5Ra) sequence linked to a transmembrane domain, and the recombinant polypeptide cannot function in signal transduction. The cellular tags can be operably linked to transgenes. The expression of the cellular tag allows identification, detection, selection, and ablation of cells expressing the transgene and the cellular tag. In some embodiments the genetically modified host cell comprises a transgene comprising a polynucleotide coding for a chimeric antigen receptor comprising a ligand binding domain, and a polynucleotide coding for a cellular tag. Pharmaceutical formulations produced by the method, and methods of using the same, are also described.

### CROSS-REFERENCE TO RELATED APPLICATION(S)

The application claims the benefit of U.S. Provisional Application No. 63/247,239, filed Sep. 22, 2021, which is hereby incorporated by reference in its entirety.

### REFERENCE TO AN ELECTRONIC SEQUENCE LISTING

The content of the electronic sequence listing (237752000640SEQLIST.xml; Size: 314,569 bytes; and Date of Creation: Sep. 21, 2022) is herein incorporated by reference in its entirety.

### FIELD

The present disclosure relates to cellular tags including an extracellular region and a transmembrane region. The cellular tags allow for identification, detection, selection and ablation of cells modified to express the cellular tags. In some embodiments, the present disclosure provides cellular tags incapable of signal transduction, which may be expressed on the surface of a cell modified to express a chimeric antigen receptor.

### BACKGROUND

The success of the CD19 CAR T cell therapies (Kymriah® marketed by Novartis Pharmaceuticals Corp., Yescarta® marketed by Kite Pharma, Inc., and Breyanzi® marketed by Juno Therapeutics, Inc.) has changed the way patients with B-cell malignancies are treated. In fact, this success is changing the way doctors and scientist are looking to treat cancer and other diseases via cell therapies. Scientists use different synthetic biology approaches to transduce a cell with a transgene that imparts the cell with a new or enhanced ability, e.g., to fight cancer. For these therapies to be effective scientist must have the ability to understand the percentage of cells that express the transgene (enrich them if the percentage is low), to know where the transgenic cells are trafficking in vivo, and, if a therapy is having an adverse effect, to ablate the transgenic cells. Cell surface markers have been used to effectively meet all or some of these criteria. A cell surface marker is usually designed from a membrane protein that is then truncated to make it relatively inert on the cell surface. The truncated protein is the cell surface tag and usually has the ability to be bound by a small molecule or antibody. CD19, CD20, CD34, CTLA-4, EGFR and HER2 are some of the surface proteins that have given rise to cell surface tags. Only tags from EGFR, HER2, CD19, and a hybrid tag of CD20 and CD34 possess all the attributes listed above and have made it into human clinical trials.

While these tags exist, the need to have more cellular tags is clear. Ideally the tag would be orthogonal to the cell type it is being used on to be a unique identifier. As cell therapies expand to use cells other than effector T cells this need becomes more pressing. Also, as multiplexing in cell therapies increases in use, so does the need to have multiple tags.

The most well-known tag is EGFRt. This coding region of this tag is roughly 1100 base-pairs in length, which encompasses a large percentage of the payload of a transgene that can effectively be transduced. Therefore, the ability to shrink the size of the coding region of the tag is important for efficacious delivery of the transgenic payload. Finally, more tags that are able to facilitate ex vivo purification of transgenic cells, monitoring of in vivo trafficking of the transgenic cells, and ablation of the transgenic cells in vivo via antibodies are needed. This invention addresses these needs.

### SUMMARY

The present disclosure provides a recombinant polypeptide comprising a cell surface tag. The tag comprises an extracellular region, a transmembrane region, and an optional intracellular region, wherein the extracellular region comprises an IL5 receptor alpha (IL5Ra) sequence linked to a transmembrane domain, wherein the recombinant polypeptide cannot function in signal transduction.

### BRIEF DESCRIPTION OF DRAWINGS

The accompanying drawings, which are incorporated herein and form a part of the specification, illustrate exemplary embodiments and, together with the description, further serve to enable a person skilled in the pertinent art to make and use these embodiments and others that will be apparent to those skilled in the art. The invention will be more particularly described in conjunction with the following drawings.

FIG. 1 shows a representation of some possible uses for cellular tags, including in vitro selection, in vivo detection and ablation of cells in a subject. The reference for flow and IHC images is Wang et al., Blood 118, 1255-1263, 2011.

FIG. 2 depicts flow cytometry plots showing expression of surface proteins (CD25, VEGFR2, and IL5Ra) in primary Treg cells from two human donors.

FIG. 3A depicts an expression cassette used to express an IL5Rat tag (pSB_0166) in K562 cells. The promoter is a human elongation factor-1 alpha promoter (EF1a). The coding region for a chimeric antigen receptor (CAR) is separated from the coding region of the IL5Ra tag by a coding region of P2A ribosome-skipping peptide. A woodchuck hepatitis virus posttranscriptional regulatory element (WPRE) is included to increase mRNA stability and protein expression. FIG. 3B-3E shows flow cytometry plots of transiently transfected and untransfected K652 cells stained with a CV-biotin that has been bound to streptavidin-FITC for CAR detection and either a commercial anti-IL5Ra-PE or benralizumab (Creative Biolab) followed by a secondary anti-human Fab-PE. FIG. 3B shows a plot of untransfected K562 control cells stained with the full antibody cocktail. FIG. 3C shows a plot of transfected K562 cells that were stained with only the anti-human IgG Fab secondary antibody. FIG. 3D shows a plot of transfected K562cells that were stained with a CV-biotin that has been bound to streptavidin-FITC for CAR detection and a commercial anti-IL5Ra-PE antibody. FIG. 3E shows a plot of transfected K562cells that were stained with a CV-biotin that has been bound to streptavidin-FITC for CAR detection and benralizumab (Creative Biolab) followed by a secondary anti-human Fab-PE for IL5Rat tag detection.

FIG. 4A depicts an expression cassette used to express IL5Rat tags and the conical EGFRt tag in Jurkat cells. FIG. 4B is a flow cytometry plot showing expression of anti-CV CAR and EGFRt in transduced Jurkat cells. FIG. 4C is a flow cytometry plot showing expression of anti-CV CAR and an IL5Rat tag (pSB_0166) in transduced Jurkat cells.

FIG. 5 shows IL5Rat tag expression on transduced Jurkat cells (pSB_0166) that were put through positive selection via the IL5Rat tag to yield a pure population of transduced cells.

FIG. 6 is a plot of an antibody-dependent cell-mediated cytotoxicity (ADCC) reporter assay showing selective ablation of transduced cells (pSB_0166) expressing an L5Rat tag.

FIG. 7A illustrates the structure of three different IL5Rat tags. FIG. 7B includes flow cytometry plots showing expression of IL5Rat tags on transduced Jurkat cells (pSB195, pSB196, or pSB198). FIG. 7C is a plot showing levels of expression of IL5Rat tags on transduced Jurkat cells. FIG. 7D is a plot of an ADCC reporter assay showing selective ablation of transduced cells expressing L5Rat tags.

FIG. 8A illustrates the structure of three different IL5Rat tags. FIG. 8B are flow cytometry plots showing expression of IL5Rat tags on transduced Jurkat cells (pSB198, pSB323, or pSB326). FIG. 8C is a plot showing levels of expression of IL5Rat tags on transduced Jurkat cells. FIG. 8D is a plot of an ADCC reporter assay showing ablation of transduced cells expressing IL5Rat tags.

FIG. 9A is a plot showing percentages of transduced Jurkat cells expressing IL5Rat tags (pSB198 or pSB590). FIG. 9B is a plot showing levels of expression of IL5Rat tags on transduced Jurkat cells. FIG. 9C is a plot of an ADCC reporter assay showing ablation of transduced cells expressing IL5Rat tags.

FIG. 10 is a plot showing binding of recombinant IL-5 to transduced Jurkat cells expressing IL5Rat tags. Transduced cells expressing IL-5Rat tags with mutant extracellular domains exhibit diminished IL-5 binding (pSB540, pSB541, pSB546, or pSB552) relative to transduced cells expressing IL-5Rat tags with wild type extracellular domains (pSB511 or pSB198).

FIG. 11A includes flow cytometry plots showing expression of IL5Rat tags and CARs on transduced Jurkat cells. FIG. 11B is a plot showing levels of expression of IL5Rat tags on transduced Jurkat cells. FIG. 11C is a plot showing percentages of transduced Jurkat cells expressing CARS and IL5Rat tags.

FIG. 12 is a plot of an ADCC reporter assay showing ablation of transduced cells expressing IL5Rat tags.

FIG. 13A includes flow cytometry plots showing expression of IL5Rat tags on transduced Jurkat cells, and a plot showing levels of expression of IL5Rat tags on transduced Jurkat cells. FIG. 13B is a plot showing binding of recombinant IL-5 to transduced Jurkat cells expressing IL5Rat tags. FIG. 13C is a plot of an ADCC reporter assay showing ablation of transduced cells expressing IL5Rat tags.

FIG. 14A is a plot showing percentages of transduced human Treg cells expressing a CAR and cell surface tag after the cells were subjected to a 14-day expansion protocol. FIG. 14B are plots showing levels of expression of IL5Rat tags on transduced human Treg cells after the cells were subjected to a 14-day expansion protocol.

### DETAILED DESCRIPTION

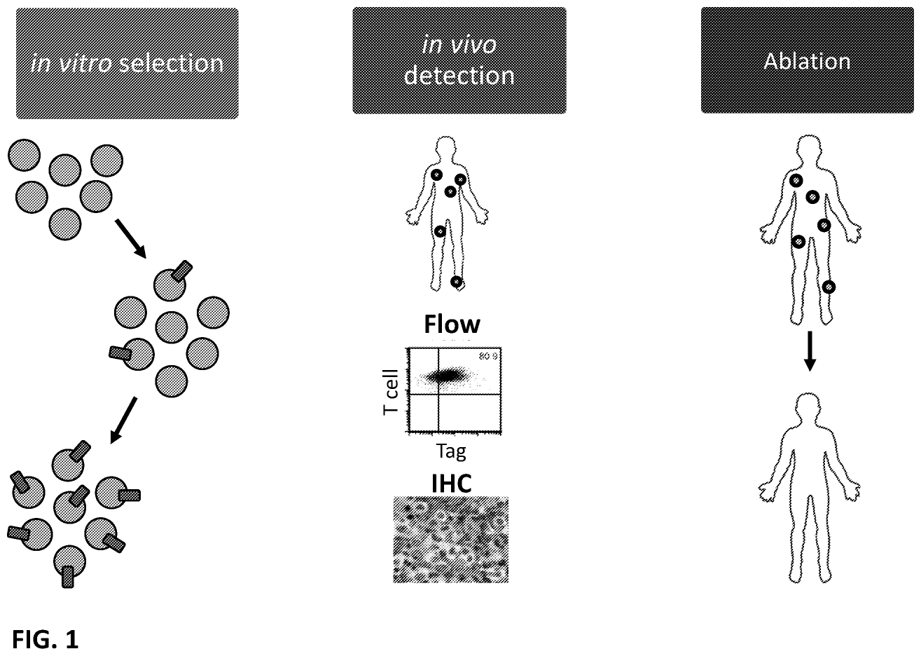

This disclosure provides novel cell surface markers that can be used for detecting, selecting, and enriching engineered cells, and for in vivo cell ablation. One aspect of the disclosure provides a genetic tag for transgene expression that provides stable expression of the transgene in cells. FIG. 1 shows some possible uses for a genetic tag in cell therapy. In some embodiments, the genetic tag provides for selection of transduced cells that express the transgene. In some embodiments, the genetic tag is expressed on the cell surface, has decreased immunogenicity, does not substantially increase the genetic payload in a vector, and/or provides for transgene expression in a variety of cells.

In some embodiments, the genetic tag is a fragment of IL-5 receptor alpha designated as IL5Rat that at least includes an epitope recognized by an anti-IL5Ra antibody. In some embodiments, the antibody specifically binds to Domain I of IL5Ra. In some embodiments, the anti-IL5Ra antibody is an antibody therapeutically useful for treating a disease or condition, e.g., cancer. In some embodiments, the epitope is recognized by benralizumab.

### Definitions

As used herein, the following meanings apply unless otherwise specified. The word “may” is used in a permissive sense (i.e., meaning having the potential to), rather than the mandatory sense (i.e., meaning must). The singular forms “a.” “an,” and “the” include plural referents. Thus, for example, reference to “an element” includes a combination of two or more elements, notwithstanding use of other terms and phrases for one or more elements, such as “one or more.” The phrase “at least one” includes “one”, “one or more”, “one or a plurality” and “a plurality”. The term “or” is, unless indicated otherwise, non-exclusive, i.e., encompassing both “and” and “or.” The term “any of” between a modifier and a sequence means that the modifier modifies each member of the sequence. So, for example, the phrase “at least any of 1, 2 or 3” means “at least 1, at least 2 or at least 3”.

Compositions or methods “comprising” or “including” one or more recited elements may include other elements not specifically recited (e.g., open-ended terms meaning including but not limited to). For example, a composition that “comprises” or “includes” a Treg cell may contain the Treg cell alone or in combination with other ingredients, such as excipients, culture medium, etc. In contrast, the phrase “consisting of” is closed, indicating that such embodiments do not include additional elements. The term “consisting essentially of” refers to the inclusion of recited elements and other elements that do not materially affect the basic and novel characteristics of a claimed combination (e.g., partially closed term). It is understood that aspects and embodiments described herein as “comprising” include “consisting of” and “consisting essentially of” embodiments.

As used herein, the terms “antigen,” “immunogen,” and “antibody target,” refer to a molecule, compound, or complex that is recognized by an antibody, i.e., can be bound by the antibody. The term can refer to any molecule that can be recognized by an antibody, e.g., a polypeptide, polynucleotide, carbohydrate, lipid, chemical moiety, or combinations thereof (e.g., phosphorylated or glycosylated polypeptides, etc.). One of skill will understand that the term does not indicate that the molecule is immunogenic in every context, but simply indicates that it can be targeted by an antibody.

As used herein, the term “epitope” refers to the localized site on an antigen that is recognized and bound by an antibody. Epitopes can include a few amino acids or portions of a few amino acids, e.g., 5 or 6, or more, e.g., 20 or more amino acids, or portions of those amino acids. In some cases, the epitope includes non-protein components, e.g., from a carbohydrate, nucleic acid, or lipid. In some cases, the epitope is a three-dimensional moiety. Thus, for example, where the target is a protein, the epitope can be comprised of consecutive amino acids, or amino acids from different parts of the protein that are brought into proximity by protein folding (e.g., a discontinuous epitope).

As used herein, the term “antibody” refers to a polypeptide comprising a framework region from an immunoglobulin gene, that specifically bind and recognize an antigen. Typically, the “variable region” contains the antigen-binding region of the antibody (or its functional equivalent) and is most critical in specificity and affinity of binding. An exemplary immunoglobulin (antibody) structural unit comprises a tetramer. Each tetramer is composed of two identical pairs of polypeptide chains, each pair having one “light” (about 25 kD) and one “heavy” chain (about 50-70 kD).

Antibodies can be of (i) any of the five major classes of immunoglobulins, based on the identity of their heavy-chain constant domains-alpha (IgA), delta (IgD), epsilon (IgE), gamma (IgG) and mu (IgM), or (ii) subclasses (isotypes) thereof (E.g., IgG1, IgG2, IgG3, IgG4, IgA1 and IgA2). The light chains can be either lambda or kappa.

The term “about” as used herein in reference to a value, encompasses from 90% to 110% of that values (e.g., about 50 amino acids, refers to 45 to 55 amino acids).

As used herein, an amino acid sequence “consists of” only the amino acids in that sequence.

As used herein, a first amino acid sequence “consists essentially of” a second amino acid sequence if the first amino acid sequence (1) comprises the second amino sequence and (2) is no more than 1, no more than 2 or no more than 3 amino acids longer than the second amino acid sequence.

As used herein, a first amino acid sequence is a “fragment” of a second amino acid sequence if the second amino acid sequence comprises the first amino acid sequence. In certain embodiments, a first amino acid sequence that is a fragment of a second amino acid sequence may have no more than any of 1, 2, 3, 4, 5, 6, 7, 8, 9 or 10 fewer amino acids than the second amino acid sequence.

As used herein, a “functional equivalent” of a reference amino acid sequence is a sequence that is not identical to the reference sequence, but that contains minor alterations such as, for example, insertion, deletion or substitution of one or a few amino acids. A functionally equivalent sequence retains the function (e.g., immunogenicity) of the reference sequence to which it is equivalent. If a functionally equivalent amino acid sequence contains substitution of one or more amino acids with respect to the reference sequence, these will generally be conservative amino acid substitutions.

As used herein, a “conservative amino acid substitution” is one in which one amino acid residue is replaced with another amino acid residue without abolishing the protein's desired properties. Suitable conservative amino acid substitutions can be made by substituting amino acids with similar hydrophobicity, polarity, and R-chain length for one another. Examples of conservative amino acid substitution include the following (Note, some categories are not mutually exclusive):

Conservative Substitutions

| Type of Amino Acid | Substitutable Amino Acids |
|---|---|
| Hydrophilic | Ala, Pro, Gly, Glu, Asp, Gln, Asn, Ser, Thr |
| Sulphydryl | Cys |
| Aliphatic (non-polar, | Ala, Val, Ile, Leu, Met, Gly, Pro |
| hydrophobic) | |
| Basic | Lys, Arg, His |
| Aromatic | Phe, Tyr, Trp |

As used herein, the term “substantially identical” refers to identity between a first amino acid sequence that contains a sufficient or minimum number of amino acid residues that are i) identical to, or ii) conservative substitutions of aligned amino acid residues in a second amino acid sequence such that the first and second amino acid sequences have a common structural domain and/or common functional activity and/or common immunogenicity. For example, amino acid sequences that contain a common structural or antigenic domain having at least about 90%, 91%, 92%, 93%, 94%, 95%, 96%, 97%, 98% or 99% identity are termed sufficiently or substantially identical. In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity, or encode polypeptides having the same immunogenic properties.

The term “sequence identity” as used herein refers to the percentage of sequence identity between two polypeptide sequences or two nucleic acid sequences. To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions times 100%). In one embodiment, the two sequences are the same length. The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules of the present application. BLAST protein searches can be performed with the XBLAST program parameters set, e.g., to score-50, wordlength=3 to obtain amino acid sequences homologous to a protein molecule described herein. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website). Another preferred, non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

Percent amino acid sequence identity may be determined using the sequence comparison program NCBI-BLAST2 (Altschul et al., Nucleic Acids Res. 25:3389-3402 (1997)). The NCBI-BLAST2 sequence comparison program may be obtained from the National Institute of Health, Bethesda, Md. NCBI-BLAST2 uses several search parameters, wherein all of those search parameters are set to default values including, for example, unmask=yes, strand=all, expected occurrences=10, minimum low complexity length=15/5, multi-pass e-value=0.01, constant for multi-pass=25, dropoff for final gapped alignment=25 and scoring matrix=BLOSUM62.

In situations where NCBI-BLAST2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program NCBI-BLAST2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

The terms “nucleic acid sequence” and “nucleotide sequence” as used herein refer to a sequence of nucleoside or nucleotide monomers consisting of naturally occurring bases, sugars and intersugar (backbone) linkages and includes cDNA. The term also includes modified or substituted sequences comprising non-naturally occurring monomers or portions thereof. The nucleic acid sequences of the present application may be deoxyribonucleic acid sequences (DNA) or ribonucleic acid sequences (RNA) and may include naturally occurring bases including adenine, guanine, cytosine, thymidine and uracil. The sequences may also contain modified bases. Examples of such modified bases include aza and deaza adenine, guanine, cytosine, thymidine and uracil; and xanthine and hypoxanthine. It is understood that polynucleotides comprising non-transcribable nucleotide bases may be useful as probes in, for example, hybridization assays. The nucleic acid can be either double stranded or single stranded, and represents the sense or antisense strand. Further, the term “nucleic acid” includes the complementary nucleic acid sequences as well as codon optimized or synonymous codon equivalents.

The term “isolated nucleic acid” as used herein refers to a nucleic acid substantially free of cellular material or culture medium when produced by recombinant DNA techniques, or chemical precursors, or other chemicals when chemically synthesized. An isolated nucleic acid is also substantially free of sequences that naturally flank the nucleic acid (i.e. sequences located at the 5′ and 3′ ends of the nucleic acid) from which the nucleic acid is derived.

As used herein, the term “expression construct” refers to a polynucleotide comprising an expression control sequence operatively linked with a heterologous nucleotide sequence (i.e., a sequence to which the expression control sequence is not normally connected to in nature) that is to be the subject of expression. As used herein, the term “expression vector” refers to a polynucleotide comprising an expression construct and sequences sufficient for replication in a host cell or insertion into a host chromosome. Plasmids and viruses are examples of expression vectors. As used herein, the term “expression control sequence” refers to a nucleotide sequence that regulates transcription and/or translation of a nucleotide sequence operatively linked thereto. Expression control sequences include promoters, enhancers, repressors (transcription regulatory sequences) and ribosome binding sites (translation regulatory sequences).

As used herein, a nucleotide sequence is “operatively linked” with an expression control sequence when the expression control sequence functions in a cell to regulate transcription of the nucleotide sequence. This includes promoting transcription of the nucleotide sequence through an interaction between a polymerase and a promoter.

The term “vector” as used herein comprises any intermediary vehicle for a nucleic acid molecule which enables said nucleic acid molecule, for example, to be introduced into prokaryotic and/or eukaryotic cells and/or integrated into a genome, and include plasmids, phagemids, bacteriophages or viral vectors such as retroviral based vectors, lentiviral vectors, Adeno Associated viral vectors and the like. The term “plasmid” as used herein generally refers to a construct of extrachromosomal genetic material, usually a circular DNA duplex, which can replicate independently of chromosomal DNA.

“Transfection” refers to the introduction of new genetic material into a cell. It includes transformation (the direct uptake and incorporation of exogenous genetic material from its surroundings through the cell membrane), transduction (the introduction of foreign DNA by a bacteriophage virus into a host cell) and conjugation.

As used herein, a “host cell” refers to a recombinant cell comprising an expression construct.

As used herein, the term “biological sample” refers to a sample containing cells (e.g., peripheral blood mononuclear cells) or biological molecules derived from cells.

As used herein, the term terms “therapy,” “treatment,” “therapeutic intervention” and “amelioration” refer to any activity resulting in a reduction in the severity of symptoms. The terms “treat” and “prevent” are not intended to be absolute terms. Treatment and prevention can refer to any delay in onset, amelioration of symptoms, improvement in patient survival, increase in survival time or rate, etc. Treatment and prevention can be complete or partial. The effect of treatment can be compared to an individual or pool of individuals not receiving the treatment, or to the same patient prior to treatment or at a different time during treatment. In some aspects, the severity of disease is reduced by at least 10%, as compared, e.g., to the individual before administration or to a control individual not undergoing treatment. In some aspects, the severity of disease is reduced by at least 25%, 50%, 75%, 80%, or 90%, or in some cases, no longer detectable using standard diagnostic techniques. “Treating” and “Treatment” can also mean prolonging survival as compared to expected survival if not receiving treatment. “Treating” and “treatment” as used herein also include prophylactic treatment.

Compositions or methods “comprising” or “including” one or more recited elements may include other elements not specifically recited (e.g., open-ended terms meaning including but not limited to). For example, a composition that “comprises” or “includes” an antibody may contain the antibody alone or in combination with other ingredients. In contrast, the phrase “consisting of” is closed, indicating that such embodiments do not include additional elements. The term “consisting essentially of” refers to the inclusion of recited elements and other elements that do not materially affect the basic and novel characteristics of a claimed combination (e.g., partially closed term). It is understood that aspects and embodiments described herein as “comprising” include “consisting of” and “consisting essentially of” embodiments.

### IL-5 Receptor Alpha

The interleukin-5 receptor is a type I cytokine receptor. It is a heterodimer of the interleukin 5 receptor alpha subunit (IL5Ra) and CSF2RB. The IL5 receptor (IL5R) belongs to the type I cytokine receptor family and is a heterodimer composed of two polypeptide chains, one a subunit, which binds IL5 and confers upon the receptor cytokine specificity, and one β subunit, which contains the signal transduction domains.

The IL5Ra chain is expressed by cosinophils, some basophils and murine B1 cells or B cell precursors. Like many other cytokine receptors, alternative splicing of the α-chain gene results in expression of either a membrane bound or soluble form of the bα-chain. The soluble form does not lead to signal transduction and therefore has an antagonistic effect on IL5 signalling. Both monomeric forms of IL5Ra are low affinity receptors, while dimerization with the β-chain produces a high affinity receptor. In either case, the α-chain exclusively binds IL5 and the intra-cellular portion of IL5Ra is associated with Janus kinase (JAK) 2, a protein tyrosine-kinase essential in IL5 signal transduction.

The present disclosure provides novel IL5Ra-derived cell surface tags. In some embodiments, these tags are truncated (i.e., not full length) IL5Ra surface proteins, that have been truncated to remove some or all the intracellular signalling domain making the protein relatively inert. In some embodiments, these proteins lack the ligand-binding and/or signal transduction functions of wild-type IL5Ra but can still be recognized by common anti-IL5Ra antibodies. In some embodiments, the extracellular domain of the IL5Rat tag can no longer bind IL5 allowing for the cell surface tag to be even more inert on the surface. However, in some variations the IL5Rat tags still have the ability to bind IL5 and still be appropriate for clinical use.

In some embodiments, the IL5Ra tags are from about 250 to 450 amino acids in length. In some embodiments, the IL5Ra tags are greater than (lower limit) about 250, 275, 300, 325, 350, 375, 400 or 425 amino acids in length. In some embodiments, the IL5Ra tags are less than (upper limit) about 450, 425, 400, 375, 350, 325, 300, or 275 amino acids in length. That is, the length is in the range of from about 250 to 450 in which the lower limit is less than the upper limit. For instance, in some embodiments, the IL5Ra tags are from about 325 to 425 amino acids in length. Unless otherwise indicated, the length range refers to the IL5Ra tag comprising a signal peptide, as opposed to a mature form of the IL5Ra tag in which the signal peptide has been removed.

In some embodiments, the IL5Rat cellular tags of the present disclosure are expressed on the cell surface and do not substantially increase the genetic payload in a vector, and/or do facilitate for transgene expression in a variety of cells.

In some embodiments, the present IL5Rat tags can be expressed at high levels on cell surface and therefore can be used as a safety switch for cell ablation in cell therapy. When the engineered cells in the therapy are no longer needed in the body, a pharmaceutical grade anti-IL5Ra antibody such as benralizumab can be administered to the patient, thereby removing the engineered cells through antibody-dependent cellular cytotoxicity (ADCC), complement-dependent cytotoxicity (CDC), and/or antibody-dependent cellular phagocytosis (ADCP). The use of benralizumab for in vivo cell ablation has the benefit that the side effects of benralizumab are very mild to the patients.

The IL5Rat tags of this disclosure can be use on all different types of cells. In some embodiments, the IL5Rat tags of this disclosure are used in Treg cells.

Unless otherwise indicated, IL5Ra is as used herein refers to human IL5Ra. A human IL5Ra polypeptide sequence may be found at the Uniprot database (Identifier No. Q01344) and may have the following sequence:

| (SEQ ID NO: 1) | |
|---|---|
| * signal peptide * # extracellular region | |
| 1 | MIIVAHVLLI LLGATEILQA DLLPDEKISL LPPVNFTIKV TGLAQVLLQW |
| 51 | KPNPDQEQRN VNLEYQVKIN APKEDDYETR ITESKCVTIL HKGFSASVRT |
| 101 | ILQNDHSLLA SSWASAELHA PPGSPGTSIV NLTCTTNTTE DNYSRLRSYQ |
| 151 | VSLHCTWLVG TDAPEDTQYF LYYRYGSWTE ECQEYSKDTL GRNIACWFPR |
| 201 | TFILSKGRDW LAVLVNGSSK HSAIRPFDQL FALHAIDQIN PPLNVTAEIE |
| 251 | GTRLSIQWEK PVSAFPIHCF DYEVKIHNTR NGYLQIEKLM TNAFISIIDD |
| #& | |
| trans- | |
| 301 | LSKYDVQVRA AVSSMCREAG LWSEWSQPIY VGNDEHKPLR EWFVIVIMAT |
| membrane &$ intracellular domain | |
| 351 | ICFILLILSL ICKICHLWIK LFPPIPAPKS NIKDLFVTTN YEKAGSSETE |
| $ | |
| 401 | IEVICYIEKP GVETLEDSVF. |

In the sequence above, the various IL5RA domains are delineated as follows. The signal peptide spans amino acids 1-20 (* . . . *). The extracellular region (SEQ ID NO:59) spans amino acids 21-342 (# . . . #), wherein Domain I (SEQ ID NO:60), Domain II (SEQ ID NO: 61), and Domain III (SEQ ID NO:62), span amino acids 32-123 (single underline), 124-242 (double underline), and 243-334 single underline), respectively. The transmembrane domain (SEQ ID NO:12) spans amino acids 343-362 (& . . . &). The intracellular domain (SEQ ID NO: 13) spans amino acids 363-420 ($ . . . $).

### IL-5 Receptor Alpha Derived Cellular Tags

The cellular tags of the present disclosure are derived from IL5Ra, comprising at least a portion of the extracellular sequence of IL5Ra. They do not comprise the entire sequence of IL5Ra, for example, they may comprise a truncated sequence of IL5Ra, for example, wherein the intracellular domain is truncated.

The cellular tags of the present disclosure are configured not to function in signal transduction. This can be accomplished by truncating the intracellular domain of IL5Ra so that it is not capable of performing signal transduction activity. It also can be accomplished by truncating the extracellular sequence of IL5Ra so that this sequence cannot bind its natural target as is necessary in signal transduction.

a. Extracellular Domain

The extracellular region of the present IL5Ra-derived cellular tag comprises an epitope bound by an anti-IL5Ra antibody. In some embodiments, the antibody is Benralizumab. By way of example, the region may comprise Domain I of IL5Ra, such as the following Domain I sequence, or a functional variant thereof:

(SEQ ID NO: 2)
PPVNFTIKVTGLAQVLLQWKPNPDQEQRNVNLEYQVKINAPKEDDYETR
ITESKCVTILHKGFSASVRTILQNDHSLLASSWASAELHAPPG.

In some embodiments, a fragment of IL5Ra comprises, consists essentially of, or consists of amino acids 32-123 (Domain I) or 32-242 (Domains I and II), or 32-334 (Domains I, II and III), or 1-334 (Domains I, II and III). An amino acid sequence “consists essentially of” a second amino acid sequence if it comprises the second amino acid sequence and no more than any of 1, 2, 3, 4, 5, 6, 7, 8, 9 or 10 additional amino acids.

In some embodiments, Domain I of IL5Ra comprises any one of the amino acid sequences listed below:

(SEQ ID NO: 67)
DLLPDEKISLLPPVNFTIKVTGLAQVLLQWKPNPDQEQRNVNLEYQVKI
NAPKEDDYETRITESKCVTILHKGFSASVRTILQNDHSLLASSWASAEL
HAPPG;
(SEQ ID NO: 68)
DLLPDEKISLLPPVNFTIKVTGLAQVLLQWKPNPDQEQRNVNLEYQVKI
NAPKEDDYETRITESKCVTALHKGFSASVRTILQNDHSLLASSWASAEL
HAPPG;
and
(SEQ ID NO: 69)
DLLPDEKISLLPPVNFTIKVTGLAQVLLQWKPNPDQEQRNVNLEYQVKI
NAPKEDDYETRITEAKCVTILHKGFSASVRTILQNDHSLLASSWASAEL
HAPPG.

In In some embodiments, Domain II of IL5Ra comprises any one of the amino acid sequences listed below:

(SEQ ID NO: 61)
SPGTSIVNLTCTTNTTEDNYSRLRSYQVSLHCTWLVGTDAPEDTQYFLY
YRYGSWTEECQEYSKDTLGRNIACWFPRTFILSKGRDWLAVLVNGSSKH
SAIRPFDQLFALHAIDQINPP;
(SEQ ID NO: 70)
SPGTSIVNLTCTTNTTEDNYSRLRSYQVSLHCTWLVGTDAPEDTQYFLY
YRYGSWTEECQEYSKDTLGRNIACWFPRTFALSKGRDWLAVLVNGSSKH
SAIRPFDQLFALHAIDQINPP;
(SEQ ID NO: 71)
SPGTSIVNLTCTTNTTEDNYSRLRSYQVSLHCTWLVGTDAPEDTQYFLY
YRYGSWTEECQEYSKDTLGRNIACWFPRTFILSAGRDWLAVLVNGSSKH
SAIRPFDQLFALHAIDQINPP;
and
(SEQ ID NO: 72)
SPGTSIVNLTCTTNTTEDNYSRLRSYQVSLHCTWLVGTDAPEDTQYFLY
YRYGSWTEECQEYSKDTLGRNIACWFPRTFILSKGADWLAVLVNGSSKH
SAIRPFDQLFALHAIDQINPP.

In some embodiments, Domain III of IL5Ra comprises any one of the amino acid sequences listed below:

(SEQ ID NO: 73)
LNVTAEIEGTRLSIQWEKPVSAFPIHCFDYEVKIHNTRNGYLQIEKLMT
NAFISIIDDLSKYDVQVRAAVSSMCREAGLWSEWSQPIYVGNDEHKPLR
EW;
(SEQ ID NO: 74)
LNVTAEIEGTRLSIQWEKPVSAFPIHCFDYEVKIHNTRNGYLQIEKLMT
NAFISIIDDLSKYDVQVRAAVSSACREAGLWSEWSQPIYVGNDEHKPLR
EW;
(SEQ ID NO: 75)
LNVTAEIEGTRLSIQWEKPVSAFPIHCFDYEVKIHNTRNGYLQIEKLMT
NAFISIIDDLSKYDVQVRAAVSSMAREAGLWSEWSQPIYVGNDEHKPLR
EW;
(SEQ ID NO: 76)
LNVTAEIEGTRLSIQWEKPVSAFPIHCFDYEVKIHNTRNGYLQIEKLMT
NAFISIIDDLSKYDVQVRAAVSSMCAEAGLWSEWSQPIYVGNDEHKPLR
EW;
and
(SEQ ID NO: 77)
LNVTAEIEGTRLSIQWEKPVSAFPIHCFDYEVKIHNTRNGYLQIEKLMT
NAFISIIDDLSKYDVQVRAAVSSMCRAAGLWSEWSQPIYVGNDEHKPLR
EW.

In some embodiments, the IL5Ra-derived cellular tag comprises a variant of the IL5Ra fragment that has at least any of 90%, 91%, 92%, 93%, 94%, 95%, 96%, 97%, 98%, or 99% sequence identity with the extracellular domain of sequence of SEQ ID NO 1 or a percentage sequence identity that is between a range defined by any two of the aforementioned percentages. In some embodiments, the variant fragment has at least any of 9, 8, 7, 6, 5, 4, 3, 2, or 1 amino acid substitutions, preferably conservative amino acid substitutions.

In some embodiments, the IL5Ra-derived cellular tag comprises a variant of the Domain I of IL5Ra that has at least any of 90%, 91%, 92%, 93%, 94%, 95%, 96%, 97%, 98%, or 99% sequence identity with the extracellular domain of sequence of SEQ ID NO 2. In some embodiments, the extracellular region may further comprise additional IL5Ra sequences such as sequences derived from Domain II and/or III. In other embodiments, the extracellular region excludes some or all sequences of Domain II and/or III.

In some embodiments, the IL5Ra-derived cellular tag comprises a variant of the Domain II of IL5Ra that has at least any of 90%, 91%, 92%, 93%, 94%, 95%, 96%, 97%, 98%, or 99% sequence identity with the extracellular domain of sequence of SEQ ID NO 61. In some embodiments, the extracellular region may further comprise additional IL5Ra sequences such as sequences derived from Domain I and/or III. In other embodiments, the extracellular region excludes some or all sequences of Domain I and/or III.

In some embodiments, the IL5Ra-derived cellular tag comprises a variant of the Domain III of IL5Ra that has at least any of 90%, 91%, 92%, 93%, 94%, 95%, 96%, 97%, 98%, or 99% sequence identity with the extracellular domain of sequence of SEQ ID NO 73. In some embodiments, the extracellular region may further comprise additional IL5Ra sequences such as sequences derived from Domain I and/or II. In other embodiments, the extracellular region excludes some or all sequences of Domain I and/or II.

In some embodiments, the IL5Ra-derived cellular tag comprises a variant of the IL5Ra fragment that has decreased binding to IL-5.

In some embodiments, the IL5Ra-derived cellular tag comprises a variant of the Domain I of IL5Ra that has at least any of 90%, 91%, 92%, 93%, 94%, 95%, 96%, 97%, 98%, or 99% sequence identity with the extracellular domain of sequence of SEQ ID NO 67, SEQ ID NO 68, or SEQ ID NO 69. In some embodiments, the extracellular region may further comprise additional IL5Ra sequences such as sequences derived from Domain II and/or III. In other embodiments, the extracellular region excludes some or all sequences of Domain II and/or III.

In some embodiments, the IL5Ra-derived cellular tag comprises a variant of the Domain II of IL5Ra that has at least any of 90%, 91%, 92%, 93%, 94%, 95%, 96%, 97%, 98%, or 99% sequence identity with the extracellular domain of sequence of SEQ ID NO 70, SEQ ID NO 71, or SEQ ID NO 72. In some embodiments, the extracellular region may further comprise additional IL5Ra sequences such as sequences derived from Domain I and/or III. In other embodiments, the extracellular region excludes some or all sequences of Domain I and/or III.

In some embodiments, the IL5Ra-derived cellular tag comprises a variant of the Domain III of IL5Ra that has at least any of 90%, 91%, 92%, 93%, 94%, 95%, 96%, 97%, 98%, or 99% sequence identity with the extracellular domain of sequence of SEQ ID NO 74, SEQ ID NO 75, SEQ ID NO 76, or SEQ ID NO 77. In some embodiments, the extracellular region may further comprise additional IL5Ra sequences such as sequences derived from Domain I and/or II. In other embodiments, the extracellular region excludes some or all sequences of Domain I and/or II.

In some embodiments, the genetic tag comprises amino acid sequences that are heterologous to IL5Ra, that is, sequences that are not native to the IL5Ra protein. One example of a heterologous sequence is a sequence of a transmembrane region from a gene other than IL5Ra.

b. Transmembrane Domain

The transmembrane region of the present polypeptides contains a hydrophobic sequence. This region may comprise an artificial sequence or may be derived from any transmembrane protein. When the source is natural, the domain can be derived from any membrane-bound or transmembrane protein. Transmembrane regions comprise for example the transmembrane region(s) of the alpha, beta or zeta chain of the T-cell receptor, CD28, CD3, CD45, CD4, CD8, CD9, CD16, CD22; CD33, CD37, CD64, CD80, CD86, CD134, CD137, CD154, members of the endothelial growth factor receptor family (EGRF/ErbB1/HER1; ErbB2/HER2/neu ErbB3/HER3; ErbB4/HER4), hepatocyte growth factor receptor (HGFR/c-MET), insulin-like growth factor receptor-1 (IGF-1R), EpCAM, VEGFR, integrins, TNF receptor superfamily (e.g., TRAILR1, TRAIL-R2), PDGF Receptor, interferon receptor, folate receptor, GPNMB, ICAM-1, HLA-DR, CEA, CA-125, MUC1, TAG-72, IL-6 receptor, 5T4, GD2, GD3, or other clusters of differentiation (e.g., CD2, CD5, CD11, CD11a/LFA-1, CD15, CD18/ITGB2, CD19, CD20, CD23/IgE Receptor, CD30, CD38, CD40, CD41, CD44, CD51, CD52, CD62L, CD74, CD125, CD147/basigin, CD152/CTLA-4, CD195/CCR5, CD319/SLAMF7). In a specific alternative, the transmembrane domain comprises the amino acid sequence of the IL5Ra transmembrane domain with the sequence of amino acids 343-362 of SEQ ID NO:1.

In a specific alternative, the transmembrane domain may be derived from any transmembrane protein, which may be, for example, CD28, EGFR, Her2, SlamF7, VEGFR2, CD34, PDGFRa, CD8, or CD4. In some embodiments, the transmembrane domain comprises any one of the amino acid sequences listed below:

(SEQ ID NO: 3)
FWVLVVVGGVLACYSLLVTVAFIIFWV;
(SEQ ID NO: 4)
IATGMVGALLLLLVVALGIGLFM;
(SEQ ID NO: 5)
SIISAVVGILLVVVLGVVFGILI;
(SEQ ID NO: 6)
VLLCLLLVPLLLSLFVLGLFL
(SEQ ID NO: 7)
IIILVGTAVIAMFFWLLLVII;
(SEQ ID NO: 8)
LIALVTSGALLAVLGITGYFL;
(SEQ ID NO: 9)
AAVLVLLVIVIISLIVLVVIW;
(SEQ ID NO: 10)
IYIWAPLAGTCGVLLLSLVIT;
and
(SEQ ID NO: 11)
MALIVLGGVAGLLLFIGLGIFF

In some embodiments, the transmembrane domain comprises any one of the amino acid sequences listed below:

(SEQ ID NO: 12)
FVIVIMATICFILLILSLIC;
(SEQ ID NO: 7)
IIILVGTAVIAMFFWLLLVII;
(SEQ ID NO: 5)
SIISAVVGILLVVVLGVVFGILI;
(SEQ ID NO: 9)
AAVLVLLVIVIISLIVLVVIW;
(SEQ ID NO: 78)
VSAVTLAYLIFCLCSLVGILHL;
(SEQ ID NO: 6)
VLLCLLLVPLLLSLFVLGLFL;
and
(SEQ ID NO: 79)
IVAVIFGLLLGAALLLGILVF

In some embodiments, synthetic or variant transmembrane domains comprise predominantly hydrophobic residues, such as leucine and valine. In some embodiments, a transmembrane domain can have at least any of 80%, 85%, 90%, 95%, or 100% amino acid sequence identity with a transmembrane domain FVIVIMATICFILLILSLIC (SEQ ID NO: 12) or percentage sequence identity that is between a range defined by any two of the aforementioned percentages. Variant transmembrane domains preferably have a hydrophobic score of at least 50 as calculated by Kyte Doolittle.

In some embodiments, a fragment of IL5Ra comprises the transmembrane domains describe above and an extracellular domain comprising amino acids 32-123 (Domain I) or 32-242 (Domains I and II), or 32-334 (Domains I, II and III).

c. Intracellular Region

In some embodiments, the present polypeptides contain an intracellular region. In some embodiments, a intracellular region can have at least any of 80%, 85%, 90%, 95%, or 100% amino acid sequence identity with an intracellular region KICHLWIKLFPPIPAPKSNIKDLFVTTNYEKAGSSETEIEVICYIEKPGVETLEDSVF (SEQ ID NO:13) or percentage sequence identity that is between a range defined by any two of the aforementioned percentages. The intracellular region of the cellular tags described herein can be 1 to 9 (e.g., 2-9, 3-9, 4-9, 5-9, 1-4, 1-5, 1-6, or 5-8) amino acids long. They also can be longer than 9 amino acids.

In some embodiments, the present polypeptides contain an intracellular region. In some embodiments, a intracellular region can have at least any of 80%, 85%, 90%, 95%, or 100% amino acid sequence identity with a intracellular region KICHLWIK (SEQ ID NO:14) or percentage sequence identity that is between a range defined by any two of the aforementioned percentages.

In some embodiments, the cytoplasmic domain comprises any one of the amino acid sequences listed below:

(SEQ ID NO: 14)
KICHLWIK;
(SEQ ID NO: 80)
LRTVKRANGG;
(SEQ ID NO: 81)
KRRQQKIRK;
(SEQ ID NO: 82)
KQKPRYEIRWRVIESIS;
(SEQ ID NO: 83)
QRALVLRRKRKR;
(SEQ ID NO: 84)
WFLKRERQ;
and
(SEQ ID NO: 85)
RSRRAQRQRQQRQRDR

In some embodiments, a fragment of IL5Ra comprises the intracellular domains describe above and an extracellular domain comprising amino acids 32-123 (Domain I) or 32-242 (Domains I and II), or 32-334 (Domains I, II and III). In some embodiments, a fragment of IL5Ra comprises, consists essentially of, or consists of amino acids 32-370 (Domains I, II and III, transmembrane domain and a fragment of the intracellular domain).

d. Signal Peptide

In some embodiments, the cellular tags described herein includes a peptide that enhances surface expression of the cellular tags. The signal peptide, also referred to herein as a signal sequence, may be derived from that of any cell surface protein or secreted protein. Such peptides include, for example, including the granulocyte macrophage stimulating factor signal sequence, endogenous HER2 leader peptide (aa 1-22), type I signal peptides, IgGK signal peptide, GM-CSFRa signal sequence and/or CD8 leader sequence. In some embodiments, the signal peptide has a sequence of: MIIVAHVLLILLGATEILQA (SEQ ID NO: 58) or MLLLVTSLLLCELPHPAFLLIP (SEQ ID NO:15). In some embodiments, the signal peptide comprises any one of the amino acid sequences listed below:

(SEQ ID NO: 58)
MIIVAHVLLILLGATEILQA;
(SEQ ID NO: 15)
MLLLVTSLLLCELPHPAFLLIP;
(SEQ ID NO: 63)
MQSKVLLAVALWLCVETRA;
(SEQ ID NO: 64)
MELAALCRWGLLLALLPPGAAS;
(SEQ ID NO: 65)
MGTSHPAFLVLGCLLTGLSLILC;.
or
(SEQ ID NO: 66)
MAGSPTCLTLIYILWQLTGSAA.

The various domains described above for the extracellular, transmembrane, and intracellular regions of the present polypeptides may be linked directly or through a peptide linker.

e. Linker Sequence

Optionally, a linker sequence can precede the cellular tag sequence and/or separate one or more functional domains (e.g. peptide to enhance surface expression, genetic tag, transmembrane domain) of the cellular tag. Linker sequences are optionally cleavable, for example, T2A sequences or IRES sequences. Cleavable linker sequences are typically placed to precede the cellular tag sequence in a nucleic acid construct. Other linker sequences are typically short peptides, of about 2 to 15 amino acids and are located between functional domains of the cellular tag including the peptide to enhance surface expression, cellular tag, and transmembrane domain. In some embodiments, the linkers are between 2, 3, 4, 5, 6, 7, 8, 9, 10, 11, 12, 13, 14 or 15 amino acids and are located between functional domains of the cellular tag including the peptide to enhance surface expression, cellular tag, and transmembrane domain. In some embodiments the linker is a cleavable linker. In some embodiments the linker is a cleavable T2A sequence. In some embodiments, the linker comprises IRES sequences.

In some embodiments, the linker comprises one of the following sequences

IgG4 hinge:
(SEQ ID NO: 16)
ESKYGPPCPPCP;
or
IgG4 hinge to CH3:
(SEQ ID NO: 17)
ESKYGPPCPPCPGQPREPQVYTLPPSQEEMTKNQVSLTCLVKGFYPSDI
AVEWESNGQPENNYKTTPPVLDSDGSFFLYSRLTVDKSRWQEGNVFSCS
VMHEALHNHYTQKSLSLSLGK;
or
IgG4 to CH2 (with optional mutations L235D,
N297Q) to CH3:
(SEQ ID NO: 18)
ESKYGPPCPPCPAPEFDGGPSVFLFPPKPKDTLMISRTPEVTCVVVDVS
QEDPEVQFNWYVDGVEVHNAKTKPREEQFQSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVESCSVMHEALHNHYTQKSLSLSLGK;
or
CD28 hinge:
(SEQ ID NO: 19)
IEVMYPPPYLDNEKSNGTIIHVKGKHLCPSPLFPGPSKP;
or
CD8:
(SEQ ID NO: 20)
TTTPAPRPPTPAPTIASQPLSLRPEACRPAAGGAVHTRGLDFACD.

In some embodiments, the linker comprises one of the following glycine-rich sequences:

GGGGS repeats (1-6 repeats or more):
(SEQ ID NO: 21)
GGGGS;
or
GGGSGGG linker:
(SEQ ID NO: 22)
GGGSGGG

f. Examples of IL5Rat Cellular Tags

In some embodiments, the present polypeptide comprises, consists of, or consists essentially of IL5Ra Domain I, Domain II, Domain III, transmembrane domain, and with or without a signal peptide, e.g. endogenous signal sequence (shown in the sequence):

(SEQ ID NO: 23)
MIIVAHVLLILLGATEILQADLLPDEKISLLPPVNFTIKVTGLAQVLLQ
WKPNPDQEQRNVNLEYQVKINAPKEDDYETRITESKCVTILHKGFSASV
RTILQNDHSLLASSWASAELHAPPGSPGTSIVNLTCTTNTTEDNYSRLR
SYQVSLHCTWLVGTDAPEDTQYFLYYRYGSWTEECQEYSKDTLGRNIAC
WFPRTFILSKGRDWLAVLVNGSSKHSAIRPFDQLFALHAIDQINPPLNV
TAEIEGTRLSIQWEKPVSAFPIHCFDYEVKIHNTRNGYLQIEKLMTNAF
ISIIDDLSKYDVQVRAAVSSMCREAGLWSEWSQPIYVGNDEHKPLREWF
VIVIMATICFILLILSLIC.

In some embodiments, the present polypeptide comprises, consists of, or consists essentially of IL5Ra Domain I, Domain II, Domain III, transmembrane domain, and with or without a signal peptide, e.g. GM-CSFRa signal sequence (shown in the sequence):

(SEQ ID NO: 24)
MLLLVTSLLLCELPHPAFLLIPDLLPDEKISLLPPVNFTIKVTGLAQVL
LQWKPNPDQEQRNVNLEYQVKINAPKEDDYETRITESKCVTILHKGFSA
SVRTILQNDHSLLASSWASAELHAPPGSPGTSIVNLTCTTNTTEDNYSR
LRSYQVSLHCTWLVGTDAPEDTQYFLYYRYGSWTEECQEYSKDTLGRNI
ACWFPRTFILSKGRDWLAVLVNGSSKHSAIRPFDQLFALHAIDQINPPL
NVTAEIEGTRLSIQWEKPVSAFPIHCFDYEVKIHNTRNGYLQIEKLMTN
AFISIIDDLSKYDVQVRAAVSSMCREAGLWSEWSQPIYVGNDEHKPLRE
WFVIVIMATICFILLILSLIC.

In some embodiments, the present polypeptide comprises, consists of, or consists essentially of IL5Ra Domain I, Domain II, Domain III, transmembrane domain, a fragment of the intracellular domain and with or without a signal peptide (shown in the sequence):

(SEQ ID NO: 25)
MLLLVTSLLLCELPHPAFLLIPDLLPDEKISLLPPVNFTIKVTGLAQVL
LQWKPNPDQEQRNVNLEYQVKINAPKEDDYETRITESKCVTILHKGFSA
SVRTILQNDHSLLASSWASAELHAPPGSPGTSIVNLTCTTNTTEDNYSR
LRSYQVSLHCTWLVGTDAPEDTQYFLYYRYGSWTEECQEYSKDTLGRNI
ACWFPRTFILSKGRDWLAVLVNGSSKHSAIRPFDQLFALHAIDQINPPL
NVTAEIEGTRLSIQWEKPVSAFPIHCFDYEVKIHNTRNGYLQIEKLMTN
AFISIIDDLSKYDVQVRAAVSSMCREAGLWSEWSQPIYVGNDEHKPLRE
WFVIVIMATICFILLILSLICKICHLWIK.

In some embodiments, the present polypeptide comprises, consists of, or consists essentially of IL5Ra Domain I, Domain II, Domain III, transmembrane domain, a fragment of the intracellular domain with C to G mutation and with or without a signal peptide, e.g., GM-CSFRa signal sequence (shown in the sequence):

(SEQ ID NO: 26)
MLLLVTSLLLCELPHPAFLLIPDLLPDEKISLLPPVNFTIKVTGLAQVL
LQWKPNPDQEQRNVNLEYQVKINAPKEDDYETRITESKCVTILHKGFSA
SVRTILQNDHSLLASSWASAELHAPPGSPGTSIVNLTCTTNTTEDNYSR
LRSYQVSLHCTWLVGTDAPEDTQYFLYYRYGSWTEECQEYSKDTLGRNI
ACWFPRTFILSKGRDWLAVLVNGSSKHSAIRPFDQLFALHAIDQINPPL
NVTAEIEGTRLSIQWEKPVSAFPIHCFDYEVKIHNTRNGYLQIEKLMTN
AFISIIDDLSKYDVQVRAAVSSMCREAGLWSEWSQPIYVGNDEHKPLRE
WFVIVIMATICFILLILSLICKIGHLWIK.

In some embodiments, the present polypeptide comprises, consists of, or consists essentially of IL5Ra Domain I, Domain II, Domain III, transmembrane domain, a fragment of the intracellular domain with an additional four amino acids at the end, and with or without a signal peptide, e.g., GM-CSFRa signal sequence (shown in the sequence):

(SEQ ID NO: 27)
MLLLVTSLLLCELPHPAFLLIPDLLPDEKISLLPPVNFTIKVTGLAQVL
LQWKPNPDQEQRNVNLEYQVKINAPKEDDYETRITESKCVTILHKGFSA
SVRTILQNDHSLLASSWASAELHAPPGSPGTSIVNLTCTTNTTEDNYSR
LRSYQVSLHCTWLVGTDAPEDTQYFLYYRYGSWTEECQEYSKDTLGRNI
ACWFPRTFILSKGRDWLAVLVNGSSKHSAIRPFDQLFALHAIDQINPPL
NVTAEIEGTRLSIQWEKPVSAFPIHCFDYEVKIHNTRNGYLQIEKLMTN
AFISIIDDLSKYDVQVRAAVSSMCREAGLWSEWSQPIYVGNDEHKPLRE
WFVIVIMATICFILLILSLICKICH.

In some embodiments, the present polypeptide comprises, consists of, or consists essentially of IL5Ra Domain I, a G₄SG₃ Linker, transmembrane domain, and with or without a signal peptide, e.g., GM-CSFRa signal sequence (shown in the sequence):

(SEQ ID NO: 28)
MLLLVTSLLLCELPHPAFLLIPDLLPDEKISLLPPVNFTIKVTGLAQVL
LQWKPNPDQEQRNVNLEYQVKINAPKEDDYETRITESKCVTILHKGFSA
SVRTILQNDHSLLASSWASAELHAPPGGGGSGGGFVIVIMATICFILLI
LSLIC.

In some embodiments, the present polypeptide comprises, consists of, or consists essentially of IL5Ra Domain I, a (G₃S)₃ Linker, transmembrane domain, and with or without a signal peptide, e.g. GM-CSFRa signal sequence (shown in the sequence):

(SEQ ID NO: 29)
MLLLVTSLLLCELPHPAFLLIPDLLPDEKISLLPPVNFTIKVTGLAQVL
LQWKPNPDQEQRNVNLEYQVKINAPKEDDYETRITESKCVTILHKGFSA
SVRTILQNDHSLLASSWASAELHAPPGGGGGSGGGGSGGGGSFVIVIMA
TICFILLILSLIC.

In some embodiments, the present polypeptide comprises, consists of, or consists essentially of IL5Ra Domain I, a IgG4 hinge Linker, transmembrane domain, and with or without a signal peptide, e.g., GM-CSFRa signal sequence (shown in the sequence):

(SEQ ID NO: 30)
MLLLVTSLLLCELPHPAFLLIPDLLPDEKISLLPPVNFTIKVTGLAQVL
LQWKPNPDQEQRNVNLEYQVKINAPKEDDYETRITESKCVTILHKGFSA
SVRTILQNDHSLLASSWASAELHAPPGESKYGPPCPPCPFVIVIMATIC
FILLILSLIC.

In some embodiments, the present polypeptide comprises, consists of, or consists essentially of IL5Ra Domain I, a IgG4 hinge Linker, transmembrane domain, and with or without a signal peptide, e.g., endogenous signal sequence (shown in the sequence):

(SEQ ID NO: 31)
MIIVAHVLLILLGATEILQADLLPDEKISLLPPVNFTIKVTGLAQVLLQ
WKPNPDQEQRNVNLEYQVKINAPKEDDYETRITESKCVTILHKGFSASV
RTILQNDHSLLASSWASAELHAPPGESKYGPPCPPCPFVIVIMATICFI
LLILSLIC.

In some embodiments, the present polypeptide comprises, consists of, or consists essentially of IL5Ra Domain I, a (G₃SG₃) Linker, transmembrane domain with an additional four amino acids at the end, and with or without a signal peptide, e.g., GM-CSFRa signal sequence (shown in the sequence):

(SEQ ID NO: 32)
MLLLVTSLLLCELPHPAFLLIPDLLPDEKISLLPPVNFTIKVTGLAQVL
LQWKPNPDQEQRNVNLEYQVKINAPKEDDYETRITESKCVTILHKGFSA
SVRTILQNDHSLLASSWASAELHAPPGGGGSGGGFVIVIMATICFILLI
LSLICKICH.

In some embodiments, the present polypeptide comprises, consists of, or consists essentially of IL5Ra Domain I, a (G3S)3 Linker, transmembrane domain with an additional four amino, and with or without a signal peptide, e.g., GM-CSFRa signal sequence (shown in the sequence):

(SEQ ID NO: 33)
MLLLVTSLLLCELPHPAFLLIPDLLPDEKISLLPPVNFTIKVTGLAQVL
LQWKPNPDQEQRNVNLEYQVKINAPKEDDYETRITESKCVTILHKGFSA
SVRTILQNDHSLLASSWASAELHAPPGGGGGSGGGGSGGGGSFVIVIMA
TICFILLILSLICKICH.

In some embodiments, the present polypeptide comprises, consists of, or consists essentially of IL5Ra Domain I, a IgG4 hinge Linker, transmembrane domain with an additional four amino, and with or without a signal peptide, e.g., GM-CSFRa signal sequence (shown in the sequence):

(SEQ ID NO: 34)
MLLLVTSLLLCELPHPAFLLIPDLLPDEKISLLPPVNFTIKVTGLAQVL
LQWKPNPDQEQRNVNLEYQVKINAPKEDDYETRITESKCVTILHKGFSA
SVRTILQNDHSLLASSWASAELHAPPGESKYGPPCPPCPFVIVIMATIC
FILLILSLICKICH.

In some embodiments, the present polypeptide comprises, consists of, or consists essentially of IL5Ra Domain I, a IgG4 hinge Linker, transmembrane domain with an additional four amino, and with or without a signal peptide, e.g., endogenous signal sequence (shown in the sequence):

(SEQ ID NO: 35)
MIIVAHVLLILLGATEILQADLLPDEKISLLPPVNFTIKVTGLAQVLLQ
WKPNPDQEQRNVNLEYQVKINAPKEDDYETRITESKCVTILHKGFSASV
RTILQNDHSLLASSWASAELHAPPGESKYGPPCPPCPFVIVIMATICFI
LLILSLICKICH.

In some embodiments, the present polypeptide comprises, consists of, or consists essentially of IL5Ra Domain I, a IgG4_CH3 hinge Linker, transmembrane domain with an additional four amino, and with or without a signal peptide, e.g., GM-CSFRa signal sequence (shown in the sequence):

(SEQ ID NO: 36)
MLLLVTSLLLCELPHPAFLLIPDLLPDEKISLLPPVNFTIKVTGLAQVL
LQWKPNPDQEQRNVNLEYQVKINAPKEDDYETRITESKCVTILHKGFSA
SVRTILQNDHSLLASSWASAELHAPPGESKYGPPCPPCPGQPREPQVYT
LPPSQEEMTKNQVSLTCLVKGFYPSDIAVEWESNGQPENNYKTTPPVLD
SDGSFFLYSRLTVDKSRWQEGNVFSCSVMHEALHNHYTQKSLSLSLGKF
VIVIMATICFILLILSLICKICH.

In some embodiments, the present polypeptide comprises, consists of, or consists essentially of IL5Ra Domain I, a IgG4_CH2 hinge (L235D)_CH3 Linker, transmembrane domain, and with or without a signal peptide, e.g., GM-CSFRa signal sequence (shown in the sequence):

(SEQ ID NO: 37)
MLLLVTSLLLCELPHPAFLLIPDLLPDEKISLLPPVNFTIKVTGLAQVL
LQWKPNPDQEQRNVNLEYQVKINAPKEDDYETRITESKCVTILHKGFSA
SVRTILQNDHSLLASSWASAELHAPPGESKYGPPCPPCPAPEFDGGPSV
FLFPPKPKDTLMISRTPEVTCVVVDVSQEDPEVQFNWYVDGVEVHNAKT
KPREEQFQSTYRVVSVLTVLHQDWLNGKEYKCKVSNKGLPSSIEKTISK
AKGQPREPQVYTLPPSQEEMTKNQVSLTCLVKGFYPSDIAVEWESNGQP
ENNYKTTPPVLDSDGSFFLYSRLTVDKSRWQEGNVFSCSVMHEALHNHY
TQKSLSLSLGKFVIVIMATICFILLILSLICKICH.

In some embodiments, the present polypeptide comprises, consists of, or consists essentially of IL5Ra Domain I, a (G3S)3_D1_IgG4) linker and hinge, transmembrane domain with an additional four amino, and with or without a signal peptide, e.g., GM-CSFRa signal sequence (shown in the sequence):

(SEQ ID NO: 38)
MLLLVTSLLLCELPHPAFLLIPDLLPDEKISLLPPVNFTIKVTGLAQVL
LQWKPNPDQEQRNVNLEYQVKINAPKEDDYETRITESKCVTILHKGFSA
SVRTILQNDHSLLASSWASAELHAPPGGGGSGGGPPVNFTIKVTGLAQV
LLQWKPNPDQEQRNVNLEYQVKINAPKEDDYETRITESKCVTILHKGFS
ASVRTILQNDHSLLASSWASAELHAPPGESKYGPPCPPCPFVIVIMATI
CFILLILSLICKICH.

In some embodiments, the present polypeptide comprises, consists of, or consists essentially of IL5Ra Domain I, a (G₄S)3_IgG4) linker and hinge, transmembrane domain with an additional four amino, and with or without a signal peptide, e.g., GM-CSFRa signal sequence (shown in the sequence):

(SEQ ID NO: 39)
MLLLVTSLLLCELPHPAFLLIPDLLPDEKISLLPPVNFTIKVTGLAQVL
LQWKPNPDQEQRNVNLEYQVKINAPKEDDYETRITESKCVTILHKGFSA
SVRTILQNDHSLLASSWASAELHAPPGGGGGSGGGGSGGGGSPPVNFTI
KVTGLAQVLLQWKPNPDQEQRNVNLEYQVKINAPKEDDYETRITESKCV
TILHKGFSASVRTILQNDHSLLASSWASAELHAPPGESKYGPPCPPCPF
VIVIMATICFILLILSLICKICH.

In some embodiments, the present polypeptide comprises, consists of, or consists essentially of IL5Ra Domain I, a ((G₄S)₃_D1_G3SG3) linker and hinge, transmembrane domain with an additional four amino, and with or without a signal peptide, e.g., GM-CSFRa signal sequence (shown in the sequence):

(SEQ ID NO: 40)
MLLLVTSLLLCELPHPAFLLIPDLLPDEKISLLPPVNFTIKVTGLAQVL
LQWKPNPDQEQRNVNLEYQVKINAPKEDDYETRITESKCVTILHKGFSA
SVRTILQNDHSLLASSWASAELHAPPGGGGGSGGGGSGGGGSPPVNFTI
KVTGLAQVLLQWKPNPDQEQRNVNLEYQVKINAPKEDDYETRITESKCV
TILHKGFSASVRTILQNDHSLLASSWASAELHAPPGGGGSGGGFVIVIM
ATICFILLILSLICKICH.

In some embodiments, the recombinant polypeptide comprises, consists of, or consists essentially of the amino acid sequence of the IL5Ra tag [IL5Rat(K186A)EC_Her2(TMIC)(S1)] of pSB_0693 with or without a signal peptide, e.g., GM-CSFRa signal sequence (shown in the sequence):

(SEQ ID NO: 208)
MLLLVTSLLLCELPHPAFLLIPDLLPDEKISLLPPVNFTIKVTGLAQVL
LQWKPNPDQEQRNVNLEYQVKINAPKEDDYETRITESKCVTILHKGFSA
SVRTILQNDHSLLASSWASAELHAPPGSPGTSIVNLTCTTNTTEDNYSR
LRSYQVSLHCTWLVGTDAPEDTQYFLYYRYGSWTEECQEYSKDTLGRNI
ACWFPRTFILSAGRDWLAVLVNGSSKHSAIRPFDQLFALHAIDQINPPL
NVTAEIEGTRLSIQWEKPVSAFPIHCFDYEVKIHNTRNGYLQIEKLMTN
AFISIIDDLSKYDVQVRAAVSSMCREAGLWSEWSQPIYVGNDEHKPLRE
WSIISAVVGILLVVVLGVVFGILIKRRQQK.

In some embodiments, the recombinant polypeptide comprises, consists of, or consists essentially of the amino acid sequence of the IL5Ra tag [IL5Rat (K186A)] of pSB_0540, with or without a signal peptide, e.g., GM-CSFRa signal sequence (shown in the sequence):

(SEQ ID NO: 174)
MLLLVTSLLLCELPHPAFLLIPDLLPDEKISLLPPVNFTIKVTGLAQVL
LQWKPNPDQEQRNVNLEYQVKINAPKEDDYETRITESKCVTILHKGFSA
SVRTILQNDHSLLASSWASAELHAPPGSPGTSIVNLTCTTNTTEDNYSR
LRSYQVSLHCTWLVGTDAPEDTQYFLYYRYGSWTEECQEYSKDTLGRNI
ACWFPRTFILSAGRDWLAVLVNGSSKHSAIRPFDQLFALHAIDQINPPL
NVTAEIEGTRLSIQWEKPVSAFPIHCFDYEVKIHNTRNGYLQIEKLMTN
AFISIIDDLSKYDVQVRAAVSSMCREAGLWSEWSQPIYVGNDEHKPLRE
WFVIVIMATICFILLILSLICKICH.

In some embodiments, the recombinant polypeptide comprises, consists of, or consists essentially of the amino acid sequence of the IL5Ra tag [IL5RatEC_Her2(TMIC)(S1))] of pSB_0590, with or without a signal peptide, e.g., GM-CSFRa signal sequence (shown in the sequence):

(SEQ ID NO: 189)
MLLLVTSLLLCELPHPAFLLIPDLLPDEKISLLPPVNFTIKVTGLAQVL
LQWKPNPDQEQRNVNLEYQVKINAPKEDDYETRITESKCVTILHKGFSA
SVRTILQNDHSLLASSWASAELHAPPGSPGTSIVNLTCTTNTTEDNYSR
LRSYQVSLHCTWLVGTDAPEDTQYFLYYRYGSWTEECQEYSKDTLGRNI
ACWFPRTFILSKGRDWLAVLVNGSSKHSAIRPFDQLFALHAIDQINPPL
NVTAEIEGTRLSIQWEKPVSAFPIHCFDYEVKIHNTRNGYLQIEKLMTN
AFISIIDDLSKYDVQVRAAVSSMCREAGLWSEWSQPIYVGNDEHKPLRE
WSIISAVVGILLVVVLGVVFGILIKRRQQK.

In some embodiments, the recombinant polypeptide comprises, consists of, or consists essentially of the amino acid sequence of the IL5Ra tag [IL5Rat(S3)] of pSB_0198, with or without a signal peptide, e.g., GM-CSFRa signal sequence (shown in the sequence):

(SEQ ID NO: 152)
MLLLVTSLLLCELPHPAFLLIPDLLPDEKISLLPPVNFTIKVTGLAQVL
LQWKPNPDQEQRNVNLEYQVKINAPKEDDYETRITESKCVTILHKGFSA
SVRTILQNDHSLLASSWASAELHAPPGSPGTSIVNLTCTTNTTEDNYSR
LRSYQVSLHCTWLVGTDAPEDTQYFLYYRYGSWTEECQEYSKDTLGRNI
ACWFPRTFILSKGRDWLAVLVNGSSKHSAIRPFDQLFALHAIDQINPPL
NVTAEIEGTRLSIQWEKPVSAFPIHCFDYEVKIHNTRNGYLQIEKLMTN
AFISIIDDLSKYDVQVRAAVSSMCREAGLWSEWSQPIYVGNDEHKPLRE
WFVIVIMATICFILLILSLICKICH.

### Nucleic Acids, and Vectors.

Another aspect of the disclosure includes nucleic acid constructs and variants thereof coding for the cellular tags as described herein.

In some embodiments, the nucleic acid codes for an amino acid sequence of a fragment IL5Ra or a variant thereof. In some embodiments, the cellular tag sequence is an IL5 receptor alpha subunit fragment as described herein. Exemplary polynucleotides encoding the truncated IL5Ra tags are set forth as SEQ ID NOs: 41-57 and 86-147. The nucleic acids include nucleic acid sequences that are codon optimized for expression in humans, degenerate sequences, and/or variant sequences.

### Vectors.

In some embodiments, a vector comprises a nucleic acid coding for a cellular tag. A nucleic acid coding for a cellular tag can be packaged in a vector as a separate construct or linked to a nucleic acid coding for a transgene. In some embodiments, a nucleic acid coding for a cellular tag is packaged in a vector as a separate construct or linked to a nucleic acid coding for a transgene.

A variety of vector combinations can be constructed to provide for efficiency of transduction and transgene expression. In some embodiments, the vector is a dual packaged or single (all in one) viral vector. In other embodiments, the vectors can include a combination of viral vectors and plasmid vectors. Other viral vectors include foamy virus, adenoviral vectors, retroviral vectors, and lentiviral vectors. In some embodiments, the vector is a lentiviral vector.

In some embodiments, a plasmid vector or a viral vector comprises a nucleic acid comprising a polynucleotide coding for a cellular tag. In some embodiments, the cellular tag comprises a polynucleotide coding for IL5Rat, and further comprises a promoter, a polynucleotide coding for a peptide to enhance surface expression and/or a polynucleotide coding for a transmembrane domain. In a specific alternative, the first nucleic acid codes for a polypeptide having a sequence of SEQ ID NO:2, SEQ ID NO:23-40, or variant thereof having at least any of 80%, 85%, 90%, 95%, or 100% sequence identity with the polypeptide, and operably linked to a promoter.

In some embodiments, a plasmid or viral vector comprises a promoter operably linked to a polynucleotide coding for a chimeric antigen receptor operably linked to a polynucleotide coding for a cellular tag. In some embodiments, the polynucleotide coding for the CAR is operably linked to the cellular tag with a self-cleavable linker.

Each element of the nucleic acid can be separated from one another with a linker sequence, for example, a self-cleaving linker such as a T2A self-cleaving sequence.

In some embodiments, IRES can be used. IRES sequences are often used in molecular biology to co-express several genes under the control of the same promoter, thereby mimicking a polycistronic mRNA. In some embodiments, several genes can be place on one plasmid with one promotor and terminator. The advantage of this technique is that molecular handling is improved.

In other embodiments, the heterogeneous (heterogeneous to the vector, e.g., lentiviral vector) nucleic acid sequence is limited by the amount of additional genetic components that can be packaged in the vector. In some embodiments, a construct contains at least two genes heterogeneous to the viral vector. In some embodiments, the construct contains at least than 4 genes heterogeneous to the viral vector. The number of genes heterogeneous to the viral vector that can be packaged in the vector can be determined by detecting the expression of one or more transgenes, and selecting vector constructs that provide for transduction of at least 10% of the cells and/or detectable expression levels of the transgene in at least 10% of the cells.

In some embodiments, a lentivirus is a dual packaged virus. A dual packaged virus contains at least one nucleic acid comprising a polynucleotide coding for a chimeric antigen receptor and a first cellular tag. Optionally the nucleic acid further comprises a polynucleotide coding for a cytokine, and/or a chemokine receptor. A dual packaged virus contains at least one nucleic acid comprising a polynucleotide coding for a chimeric antigen receptor and a second cellular tag. Optionally the nucleic acid further comprises a polynucleotide coding for a cytokine, and/or a chemokine receptor. In some embodiments of a system with two constructs, each construct can be packaged in a separate viral vector and the viral vectors can be mixed together for transduction in a cell population. In some embodiments, the first and second cellular tags are different from one another. In some embodiments, the dual packaged virus provides for expression of at least two different transgenes, (e.g. CAR constructs) in a single cell type. Using different cellular tags provides for selection of dual transduced cells.

In some embodiments, the vector is a minicircle. Minicircles are episomal DNA vectors that are produced as circular expression cassettes devoid of any bacterial plasmid DNA backbone. Their smaller molecular size enables more efficient transfections and offers sustained expression over a period of weeks as compared to standard plasmid vectors that only work for a few days. In some embodiments, a minicircle comprises a promoter linked to a polynucleotide coding for a chimeric antigen receptor operably linked to a cellular tag. One or more minicircles can be employed. In some embodiments, a minicircle comprises a promoter linked to a polynucleotide coding for a chimeric antigen receptor and first cellular tag, another minicircle comprises a promoter linked to a polynucleotide coding for a chimeric antigen receptor and a second and different cellular tag. In some embodiments, each element of the constructs is separated by a nucleic acid, such as one coding for a self-cleaving T2A sequence. In some embodiments, each minicircle differs from one another in the chimeric antigen receptor including but not limited to the spacer length and sequence, the intracellular signalling domain, and/or the cellular tag sequence.

In some embodiments, the vector is a PiggyBac transposon. The PiggyBac (PB) transposon is a mobile genetic element that efficiently transposes between vectors and chromosomes via a “cut and paste” mechanism. During transposition, the PB transposase recognizes transposon-specific inverted terminal repeat sequences (ITRs) located on both ends of the transposon vector and efficiently moves the contents from the original sites and efficiently integrates them into TTAA chromosomal sites. The powerful activity of the PiggyBac transposon system enables genes of interest between the two ITRs in the PB vector to be easily mobilized into target genomes.

In some embodiments, a PB contains a promoter linked to a polynucleotide coding for a chimeric antigen receptor operably linked to a genetic tag. One or more PB transposons can be employed. In some embodiments, a PB comprises a promoter linked to a polynucleotide coding for a chimeric antigen receptor and a first genetic tag, another PB comprises a promoter linked to a polynucleotide coding for a chimeric antigen receptor, and a second and different cellular tag. Each element of the constructs is separated by a nucleic acid, such as that coding for a self-cleaving T2A sequence. In some embodiments, each PB differs from one another in the chimeric antigen receptor including but not limited to the spacer length and sequence, the intracellular signalling domain, and/or the cellular tag sequence.

In some embodiments, a first nucleic acid comprises a first promoter operably linked to a polynucleotide coding for chimeric antigen receptor comprising a ligand binding domain, wherein the ligand binding domain binds to a ligand, wherein the ligand is a disease specific molecule, viral molecule, or any other molecule expressed on a target cell population that is suitable to mediate recognition by a lymphocyte; a polynucleotide coding for a polypeptide spacer, wherein the spacer provides for increased T cell proliferation and/or cytokine production in response to the ligand as compared to a reference chimeric receptor; a polynucleotide coding for a transmembrane domain; and d) a polynucleotide coding for an intracellular signalling domain. In some embodiments, the first nucleic acid further comprises a cellular tag.

In some embodiments, a second nucleic acid comprises a polynucleotide coding for a second and different chimeric antigen receptor. The first and second chimeric antigen receptor can differ from one another in the ligand binding domain, the target antigen, an epitope of the target antigen, the spacer domain in length and sequence (short medium or long), and in the intracellular signalling domains. In some embodiments, the second nucleic acid further comprises a second and different cellular tag from that of the first nucleic acid.

In some embodiments, in a single lentivirus construct the first and second nucleic acids can be separated by a genomic insulator nucleic acid such as the sea urchin insulator chromatin domain.

In some embodiments, promoters used herein can be inducible or constitutive promoters. Inducible promoters include a tamoxifen inducible promoter, tetracycline inducible promoter, and doxocycline inducible promoter. Constitutive promoters include SV40, CMV, UBC, EF1 alpha, PGK, and CAGG.

One or more of these vectors can be used in conjunction with one another to transduce target cells and provide for expression of a chimeric antigen receptor.

### Transgenes

Several transgenes are also aspects of the invention. The cellular tags as described herein are useful for the selection, tracking, and killing of cells transduced with and expressing a transgene. The cellular tags can be utilized with any number of different transgenes. In this disclosure, chimeric antigen receptor transgenes are exemplified but similar principals apply to the design, identification and selection of other transgenes expressed in transduced cells.

In some embodiments, the transgene expresses an antigen receptor and/or another additional polypeptide. The antigen receptor may be, for example, an antibody, an engineered antibody such as an scFv, a CAR, an engineered TCR, a TCR mimic or a chimeric antibody-T cell receptor, or a chimeric signaling receptor. The antigen receptor may target an antigen of interest (e.g., a tumor antigen or an antigen of a pathogen). The antigens may include, without limitation, AFP (alpha-fetoprotein), αvβ6 or another integrin, BCMA, B7-H3, B7-H6, CA9 (carbonic anhydrase 9), CCL-1 (C-Cmotif chemokine ligand 1), CD5, CD19, CD20, CD21, CD22, CD23, CD24, CD30, CD33, CD38, CD40, CD44, CD44v6, CD44v7/8, CD45, CD47, CD56, CD66c, CD70, CD74, CD79a, CD79b, CD98, CD123, CD138, CD171, CD352, CEA (carcinoembryonic antigen), Claudin, c-MET, DLL3 (delta-like protein 3), DLL4, ENPP3 (ectonucleotide pyrophosphatase/phosphodiesterase family member 3), EpCAM, EPG-2 (epithelial glycoprotein 2), EPG-40, ephrinB2, EPHa2 (ephrine receptor A2), ERBB dimers, estrogen receptor, ETBR (endothelin B receptor), FAP-α (fibroblast activation protein α), fetal AchR (fetal acetylcholine receptor), FBP (a folate binding protein), FCRL5, FR-α (folate receptor alpha), GCC (guanyl cyclase C), GD2, GD3, GPC2 (glypican-2), GPC3, gp100 (glycoprotein 100), GPNMB (glycoprotein NMB), GPRC5D (G Protein Coupled Receptor 5D), HER2, HER3, HER4, hepatitis B surface antigen, HLA-A1 (human leukocyte antigen A1), HLA-A2 (human leukocyte antigen A2), HMW-MAA (human high molecular weight-melanoma-associated antigen), IGFIR (insulin-like growth factor 1 receptor), Ig kappa, Ig lambda, IL-22Ra (IL-22 receptor alpha), IL-13Ra2 (IL-13 receptor alpha 2), KDR (kinase insert domain receptor), LI cell adhesion molecule (LI-CAM), Liv-1, LRRC8A (leucine rich repeat containing 8 Family member A), Lewis Y, melanoma-associated antigen (MAGE)-A1, MAGE-A3, MAGE-A6, MART-1 (melan A), murine cytomegalovirus (MCMV), MCSP (melanoma-associated chondroitin sulfate proteoglycan), mesothelin, mucin 1 (MUC1), MUC16, MHC/peptide complexes (e.g., HLA-A complexed with peptides derived from AFP, KRAS, NY-ESO, MAGE-A, and WT1), NCAM (neural cell adhesion molecule), Nectin-4, NKG2D (natural killer group 2 member D) ligands, NY-ESO, oncofetal antigen, PD-1, PD-L1, PRAME (preferentially expressed antigen of melanoma), progesterone receptor, PSA (prostate specific antigen), PSCA (prostate stem cell antigen), PSMA (prostate specific membrane antigen), ROR1, ROR2, SIRPa (signal-regulatory protein alpha), SLIT. SLITRK6 (NTRK-like protein 6), STEAP1 (six transmembrane epithelial antigen of the prostate 1), survivin, TAG72 (tumor-associated glycoprotein 72), TPBG (trophoblast glycoprotein), Trop-2, VEGFR1 (vascular endothelial growth factor receptor 1), VEGFR2, and antigens from HIV, HBV, HCV, HPV, and other pathogens.

### Host Cells and Compositions: T Lymphocyte Populations.

The compositions described herein provide for genetically modified host cells with the vectors and/or constructs as described herein. In some embodiments, the host cells are CD4+ and/or CD8+T lymphocytes. In some embodiments, the host cells are Treg cells. In some embodiments, the host cells are precursor T cells. In some embodiments, the host cells are hematopoietic stem cells.

T lymphocytes can be collected in accordance with known techniques and enriched or depleted by known techniques such as affinity binding to antibodies such as flow cytometry and/or immunomagnetic selection. After enrichment and/or depletion steps, in vitro expansion of the desired T lymphocytes can be carried out in accordance with known techniques or variations thereof that will be apparent to those skilled in the art. In some embodiments, the T cells are autologous T cells obtained from the patient.

The T lymphocytes expanded include CD8+ cytotoxic T lymphocytes (CTL) and CD4+ helper T lymphocytes that can be specific for an antigen present on a human tumor or a pathogen. The T lymphocytes expanded include Treg cells.

In some embodiments, the expansion method can further comprise the step of adding anti-CD3 and/or anti CD28 antibody to the culture medium. Optionally, the expansion method can further comprise the step of adding IL-2 and/or IL-15 to the culture medium.

After isolation of T lymphocytes both cytotoxic and helper T lymphocytes can be sorted into naive, memory, effector T cell and Treg cell subpopulations either before or after expansion.

### Compositions

The disclosure provides for an adoptive cellular immunotherapy composition comprising a genetically modified cell preparation as described herein, e.g., genetically modified lymphocyte cells preparation. These cells are, for example, multipotent cells such as hematopoietic stem cells, various progenitor or precursor cells of hematopoietic lineages, and various immune cells (e.g., human autologous or allogeneic T, natural killer (NK), dendritic, or B cells). These cells may also be pluripotent stem cells (PSCs) such as human embryonic stem cells and induced PSCs, which can be used to generate therapeutic cell populations. In some embodiments, pluripotent and multipotent cells are differentiated into a desired cell type in vitro before being implanted into the patient.

In some embodiments, the genetically modified cell preparation is a T lymphocyte cell preparation. In some embodiments the T lymphocyte cell preparation comprises CD4+ T cells that have a chimeric receptor comprising an extracellular antibody variable domain specific for a ligand associated with the disease or disorder, a spacer region, a transmembrane domain, and an intracellular signalling domain of a T cell receptor and a cellular tag as described herein. In other embodiments, an adoptive cellular immunotherapy composition further comprises a chimeric receptor modified CD8+ cytotoxic T lymphocyte cell preparation that provides a cellular immune response, wherein the cytotoxic T lymphocyte cell preparation comprises CD8+ T cells that have a chimeric receptor comprising an extracellular single chain antibody specific for a ligand associated with the disease or disorder, a spacer region, a transmembrane domain, and an intracellular signalling domain of a T cell receptor and a cellular tag as described herein. In some embodiments, the chimeric receptor modified T cell population of the disclosure can persist in vivo for at least about 3 days or longer. In alternative each of these populations can be combined with one another or other cell types to provide a composition. In some embodiments, the host cells are Treg cells.

Embodiments include CD4 and/or CD8 host cells as described herein. In some embodiments, a host cell comprises an isolated nucleic acid, such as a nucleic acid coding for an isolated polypeptide comprising at least 95% sequence identity to a IL5Rat polypeptide having a sequence of amino acids 32 to 123, or 32 to 242, or 32 to 334, or 32 to 370 of SEQ ID NO: 1 linked to a transmembrane domain, wherein the isolated polypeptide specifically binds to an antibody that binds to an epitope in Domain I of IL5Ra, and a second nucleic acid coding for a second chimeric antigen receptor and a second cellular tag. In some embodiments, the host cells are Treg cells.

In other embodiments, a composition comprises a first host cell comprising a first isolated nucleic acid, such as a nucleic acid coding for an isolated polypeptide comprising at least 95% sequence identity to a IL5Rat polypeptide having a sequence of amino acids 32 to 123, or 32 to 242, or 32 to 334, or 32 to 370 of SEQ ID NO: 1 linked to a transmembrane domain, wherein the isolated polypeptide specifically binds to an antibody that binds to an epitope in Domain I of IL5Ra, and a second host cell comprising a second nucleic acid coding for a second chimeric antigen receptor and a second cellular tag. In some embodiments, the first host cell and the second host cell can be the same or different type of host cells, for example, the first host cell can be a CD8 cell, and the second host cell can be a CD4 cell. In some embodiments, first and second host cells are each selected from the group consisting of CD8 T cells, CD4 T cells, CD4 naïve T cells, CD8 naive T cells, CD8 central memory cells, CD4 central memory cells, Treg cells and combinations thereof.

In some embodiments, the CD4+T helper lymphocyte cell is selected from the group consisting of naive CD4+ T cells, central memory CD4+ T cells, effector memory CD4+ T cells, or bulk CD4+ T cells. In some embodiments, CD4+ helper lymphocyte cell is a naive CD4+ T cell, wherein the naive CD4+ T cell comprises a CD45RO−, CD45RA+, CD62L+ CD4+ T cell.

In some embodiments, the CD8+T cytotoxic lymphocyte cell is selected from the group consisting of naive CD8+ T cells, central memory CD8+ T cells, effector memory CD8+ T cells or bulk CD8+ T cells. In some embodiments, the CD8+ cytotoxic T lymphocyte cell is a central memory T cell wherein the central memory T cell comprises a CD45RO+, CD62L+, CD8+ T cell. In yet other embodiments, the CD8+ cytotoxic T lymphocyte cell is a central memory T cell and the CD4+ helper T lymphocyte cell is a naïve or central memory CD4+ T cell.

In some embodiments, the Treg cells are CD4+CD25+CD12710.

### Methods

The disclosure provides methods of making adoptive immunotherapy compositions and uses or methods of using these compositions for performing cellular immunotherapy in a subject having a disease or disorder.

Embodiments include methods of manufacturing compositions comprising host cells as described herein. In some embodiments, a method comprises introducing an isolated nucleic acid, such as a nucleic acid coding for isolated polypeptide comprising at least 95% sequence identity to a IL5Ra polypeptide having a sequence of amino 32 to 123, or 32 to 242, or 32 to 334, or 32 to 370 of SEQ ID NO:1 linked to a transmembrane domain, wherein the isolated polypeptide specifically binds to an antibody that binds to an epitope in Domain I of IL5Ra, into a host cell; and culturing the host cells in a medium comprising at least one growth factor. In some embodiments, a method further comprises selecting the host cells for expression of IL5Rat before or after or both before and after the culturing step. In other embodiments, a method of manufacturing further comprises introducing a second nucleic acid coding for a second chimeric antigen receptor and a second cellular tag into the host cell. In some embodiments, the method further comprises selecting the host cells for expression of the second cellular tag before or after or both before and after the culturing step. In some embodiments, the host cells are T cells. In some embodiments, the host cells are Treg cells.

In other embodiments, a method comprises introducing a first isolated nucleic acid, such as a nucleic acid coding for isolated polypeptide comprising at least 95% sequence identity to a IL5Ra polypeptide having a sequence of amino acids 32 to 123, or 32 to 242, or 32 to 334, or 32 to 370 of SEQ ID NO: 1 linked to a transmembrane domain, wherein the isolated polypeptide specifically binds to an antibody that binds to an epitope in Domain I of IL5Ra, into a first host cell; selecting first host cells that express IL5Rat, introducing a second nucleic acid coding for a second chimeric antigen receptor and a second cellular tag into a second host cell, selecting second host cells for expression of the second cellular tag, and optionally, culturing the first and second host cells in a medium comprising at least one growth factor. In some embodiments, a composition comprises a first and second host cell population.

In some embodiments, a method comprises introducing an isolated nucleic acid, such as a nucleic acid coding for isolated polypeptide comprising at least 95% sequence identity to a IL5Ra polypeptide having a sequence of amino 32 to 123, or 32 to 242, or 32 to 334, or 32 to 370 of SEQ ID NO: 1 linked to a transmembrane domain, wherein the isolated polypeptide has decreased binding to IL-5, into a host cell; and culturing the host cells in a medium comprising at least one growth factor. In some embodiments, the isolated polypeptide comprises a variant of the Domain 1 of IL5Ra having the amino acid sequence of SEQ ID NO 67, SEQ ID NO 68, or SEQ ID NO 69. In some embodiments, the isolated polypeptide comprises a variant of the Domain 2 of IL5Ra having the amino acid sequence of SEQ ID NO 70, SEQ ID NO 71, or SEQ ID NO 72. In some embodiments, the isolated polypeptide comprises a variant of the Domain 3 of IL5Ra having the amino acid sequence of SEQ ID NO 74, SEQ ID NO 75, SEQ ID NO 76, or SEQ ID NO 77. In some embodiments, a method further comprises selecting the host cells for expression of IL5Rat before or after or both before and after the culturing step. In other embodiments, a method of manufacturing further comprises introducing a second nucleic acid coding for a second chimeric antigen receptor and a second cellular tag into the host cell. In some embodiments, the method further comprises selecting the host cells for expression of the second cellular tag before or after or both before and after the culturing step. In some embodiments, the host cells are T cells. In some embodiments, the host cells are Treg cells.

In some embodiments, the disclosure provides a method of manufacturing the compositions comprises obtaining a modified naïve, central memory or regulatory CD4+ T cell, wherein the modified CD4+T lymphocyte cell preparation comprises CD4+ T cells that have a chimeric receptor comprising a ligand binding domain specific for an antigen associated with a disease, a spacer domain, a transmembrane domain, and an intracellular signalling domain and a cellular tag as described herein.

In another embodiments, the disclosure provides a method comprises obtaining a modified CD8+ T cell, wherein the CD8 T lymphocyte cell preparation comprises CD8+ cells that have a chimeric receptor comprising a ligand binding domain specific for an antigen associated with a disease, a spacer domain, a transmembrane domain, and an intracellular signalling domain and a cellular tag as described herein. In other embodiments, CD8+ cells have a cytokine or chemokine receptor under the control of an inducible promoter.

The preparation of the cells that are modified with a chimeric receptor has been described above as well as in the examples. Cells can be obtained from a patient having the disease or disorder or by a healthy donor. Cells be prepared by in vitro stimulation of T lymphocytes in the presence of antigen. Subpopulations of cells can also be isolated as described herein and combined in the methods of manufacturing. Cell populations are advantageously selected for expression of the IL5Ra tags described herein.

The disclosure also provides methods of performing cellular immunotherapy in a subject having a disease or disorder comprising administering a composition of cells (e.g. lymphocytes) expressing one or more chimeric antigen receptor and cellular tag as described herein. In some embodiments, a method of performing cellular immunotherapy in a subject having a disease or disorder is provided, wherein the method comprises administering a composition of cells expressing one or more chimeric antigen receptor and cellular tag.

In some embodiments, if the modified cells are no longer desired in a subject (e.g. a patient having a disease or disorder) an antibody that binds the cellular tag is administered. The antibody can bind to and kill the modified cells of the composition, e.g. in order to avoid toxic and/or fatal side effects. In some embodiments, the antibody or antigen binding fragment preferable contains a Fc fragment in order to activate an immune reaction such as ADCC, ADCP or CDC reactions. In other embodiments, the antibody or antigen binding fragment is conjugated to a cytotoxic agent. Cytotoxic agents include cantansinoids, calicheamicin and/or auristatins. In some embodiments, the cytotoxic agents comprise cantansinoids, calicheamicin and/or auristatins.

In some embodiments, an antibody is detectably labelled in order to allow tracking of the modified cells in vivo. In some embodiments, when the antibody is used for detection in vivo, it is preferred that the antibody or antigen binding fragment lacks all or a portion of the Fc region in order to avoid ADCC reactions. Detectable labels include biotin, His tags, myc tags, radiolabels, and/or fluorescent labels. In some embodiments the detectable labels comprise biotin, His tags, myc tags, radiolabels, and/or fluorescent labels.

Subjects that can be treated by the present invention are, in general, human and other primate subjects, such as monkeys and apes for veterinary medicine purposes. The subjects can be male or female and can be any suitable age, including infant, juvenile, adolescent, adult, and geriatric subjects. In some embodiments, the subject is a primate subject or a human.

Cells prepared as described above can be utilized in methods and compositions for adoptive immunotherapy in accordance with known techniques, or variations thereof that will be apparent to those skilled in the art based on the instant disclosure.

A therapeutically effective number of modified cells are administered to the subject. As used herein, the term “therapeutically effective” refers to a number of cells or amount of pharmaceutical composition that is sufficient, when administered to a subject suffering from or susceptible to a disease, disorder, and/or condition, to treat, prevent, and/or delay the onset or progression of the symptom(s) of the disease, disorder, and/or condition. The number of cells will depend upon the ultimate use for which the composition is intended, as will the type of cells included therein. For example, if cells that are specific for a particular antigen are desired, then the population will contain greater than 70%, generally greater than 80%>, 85% and 90-95% of such cells or any percent amount of cells within a range defined by any two of the aforementioned percentages.

The modified cells can be administered by a single infusion, or by multiple infusions over a range of time. However, since different individuals are expected to vary in responsiveness, the type and amount of cells infused, as well as the number of infusions and the time range over which multiple infusions are given are determined by the attending physician, and can be determined by routine examination.

In some embodiments, the composition as described herein are administered intravenously, intraperitoneally, intratumorly, into the bone marrow, into the lymph node, and/or into cerebrospinal fluid. In some embodiments, the chimeric receptor engineered compositions are delivered to the site of disease, e.g., tumor or inflammation site. In some embodiments, the compositions as described herein are administered with chemotherapeutic agents and/or immunosuppressants.

### Exemplary Embodiments

1. A recombinant polypeptide comprising an extracellular region, a transmembrane region, and an optional intracellular region, wherein the extracellular region comprises an IL5 receptor alpha (IL5Ra) sequence linked to a transmembrane domain, wherein the recombinant polypeptide cannot function in signal transduction.
2. The recombinant polypeptide of embodiment 1, wherein the recombinant polypeptide comprises an amino acid sequence having at least any of 80%, 85%, 90%, 95%, or 100% sequence identity with amino acids 32-123 (Domain I) of SEQ ID NO:1.
3. The recombinant polypeptide of embodiment 2, wherein the recombinant polypeptide further comprises an amino acid sequence having at least any of 80%, 85%, 90%, 95%, or 100% sequence identity with amino acids 124-242 (Domains II) of SEQ ID NO:1.
4. The recombinant polypeptide of embodiments 2 or 3, wherein the recombinant polypeptide further comprises an amino acid sequence having at least any of 80%, 85%, 90%, 95%, or 100% amino acids sequence identity with 243-334 (Domain III) of SEQ ID NO:1.
5. The recombinant polypeptide of embodiment 1, wherein extracellular region comprises a truncated IL5Ra extracellular region comprising no more than some or all of Domain I, no more than some or all of Domain II and/or no more than some or all of Domain III of IL5Ra.
6. The recombinant polypeptide of embodiment 1, wherein extracellular region comprises a truncated IL5Ra extracellular region comprising some or all of Domain I, but excluding Domain II and/or Domain III of IL5Ra
7. The recombinant polypeptide of embodiment 1, wherein the extracellular region binds benralizumab.
8. The recombinant polypeptide of any preceding embodiment, wherein the transmembrane region comprises an amino acid sequence having at least any of 80%, 85%, 90%, 95%, or 100% amino acid sequence identity with sequences 343-362 of SEQ ID NO:1.
9. The recombinant polypeptide of any one of embodiments 1 to 7, wherein the transmembrane region comprises an amino acid sequence having at least any of 80%, 85%, 90%, 95%, or 100% amino acids sequence identity with amino acid sequence selected from SEQ ID NO:3-12.
10. The recombinant polypeptide of any preceding embodiment, wherein the recombinant polypeptide comprises an intracellular domain.
11. The recombinant polypeptide of embodiment 10 wherein the intracellular domain comprises a truncated portion of the intracellular domain of IL5Ra, which truncated portion has lost signal transduction ability.
12. The recombinant polypeptide of embodiment 10 wherein the intracellular domain consists of amino acids 363-370 of SEQ ID NO:1.
13. The recombinant polypeptide of embodiments 10 or 12, wherein the intracellular domain comprises amino acids 363-366 of SEQ ID NO:1.
14. The recombinant polypeptide of embodiment 1, wherein the recombinant polypeptide comprises an amino acid sequence having at least any of 80%, 85%, 90%, 95%, or 100% sequence identity with amino acids 32-123 (Domain I) or 32-242 (Domains I and II), or 32-334 (Domains I, II and III) of SEQ ID NO:1.
15. The recombinant polypeptide of embodiment 1, wherein the recombinant polypeptide comprises an amino acid sequence having at least any of 80%, 85%, 90%, 95%, or 100% sequence identity with amino acids 32-370 (Domains I, II and III, transmembrane domain and a fragment of the intracellular domain) of SEQ ID NO:1.
16. The recombinant polypeptide of embodiment 1, wherein the recombinant polypeptide comprises an amino acid sequence having at least 95% sequence identity to an IL5Ra polypeptide having a sequence of amino acids 32 to 123, or 32 to 242, or 32 to 334, or 32 to 370 of SEQ ID NO:1.
17. The recombinant polypeptide of any preceding embodiment, further comprising a signal peptide.
18. The recombinant polypeptide of embodiment 17, wherein the signal peptide comprises an amino acid sequence having at least any of 80%, 85%, 90%, 95%, or 100% amino acids sequence identity with amino acid sequence consisting of SEQ ID NO:58, and SEQ ID NO:15.
19. The recombinant polypeptide of any preceding embodiment, further comprising a linker.
20. The recombinant polypeptide of embodiment 19, wherein the linker comprises an amino acid sequence having at least any of 80%, 85%, 90%, 95%, or 100% amino acids sequence identity with amino acid sequence selected from SEQ ID NO:16-22.
21. The recombinant polypeptide of embodiment 1, comprising an intracellular region, wherein the intracellular region has an amino acid sequence consisting or consisting essentially of SEQ ID NO:13, SEQ ID NO:14; or an amino acid sequence at least any of 80%, 85%, 90%, or 95% identical thereto.
22. The recombinant polypeptide of embodiment 1, having an amino acid sequence comprising, consisting essentially of, or consisting of any of SEQ ID NOs: 23-40; or an amino acid sequence at least any of 80%, 85%, 90%, or 95% identical thereto.
23. The recombinant polypeptide of embodiment 1, comprising at least a portion that is heterologous to IL5 Ra.
24. A nucleic acid molecule comprising a coding sequence for the recombinant polypeptide of any one of the preceding embodiments.
25. The nucleic acid molecule of embodiment 24, comprising a nucleotide sequence having at least any of 80%, 85%, 90%, 95% or 100% sequence identity to any of SEQ ID NOs: 41-57.
26. The nucleic acid molecule of embodiment 24, further comprising a coding sequence for a chimeric antigen receptor (CAR).
27. The nucleic acid molecule of embodiment 26, wherein the coding sequences for the recombinant polypeptide and the CAR are operably linked to the same promoter such that the two coding sequences are co-transcribed.
28. The nucleic acid molecule of any one of embodiments 24-27, wherein the nucleic acid molecule is a viral vector, optionally a lentiviral or retroviral vector.
29. A cell comprising the nucleic acid molecule of any one of embodiments 24-28.
30. The cell of embodiment 29, wherein the cell is a human T cell.
31. The cell of embodiment 30, wherein the cell is a human Treg cell.
32. A pharmaceutical composition comprising the cell of embodiments 29 to 31, or the nucleic acid molecule of any one of embodiments 21-28; and a pharmaceutically acceptable carrier.
33. A method of treating a patient in need thereof, comprising administering the cell of embodiments 29 to 31 to the patient, optionally wherein the cell is derived from the patient.
34. The method of embodiment 33, comprising administering the cell of embodiments 30 or 31 to the patient, wherein the cell expresses a CAR specific for an antigen present in a disease.
35. The method of embodiment 33 or 34, further comprising administering to the patient an effective amount of an antibody specific for IL5Ra once the patient has been treated, wherein the antibody elicits cytotoxicity against cells expressing the recombinant polypeptide, and optionally the antibody is IgG1 or IgG2.
36. A method of making a genetically engineered human cell, comprising providing an isolated human cell, and introducing the nucleic acid molecule of any one of embodiments 24-28 into the human cell.
37. The method of embodiment 36, wherein the human cell is a human T cell.
38. The method of embodiment 37, wherein the human cell is a human Treg cell.
39. A population of cells comprising a nucleic acid encoding a recombinant polypeptide comprising an extracellular region, a transmembrane region, and an optional intracellular region, wherein the extracellular region comprises an IL5Ra sequence linked to a transmembrane domain.
40. The population of embodiment 39, wherein the recombinant polypeptide comprises an amino acid sequence having at least any of 80%, 85%, 90%, 95%, or 100% sequence identity with amino acids 32-123 (Domain I) or 32-242 (Domains I and II), or 32-334 (Domains I, II and III) of SEQ ID NO:1.
41. The population of embodiment 39, wherein the recombinant polypeptide comprises an amino acid sequence having at least any of 80%, 85%, 90%, 95%, or 100% sequence identity with amino acids 32-370 (Domains I, II and III, transmembrane domain and a fragment of the intracellular domain) of SEQ ID NO:1.
42. The population of embodiment 39, wherein the recombinant polypeptide comprises an amino acid sequence having at least 95% sequence identity to an IL5Ra polypeptide having a sequence of amino acids 32 to 123, or 32 to 242, or 32 to 334, or 32 to 370 of SEQ ID NO:1.
43. The population of embodiment 39, wherein the recombinant polypeptide comprises SEQ ID NO:23-40; or an amino acid sequence at least any of 80%, 85%, 90%, or 95% identical thereto.
44. The population of embodiment 39, wherein the nucleic acid further comprising a coding sequence for a CAR.
45. The population of embodiment 44, wherein the coding sequences for the recombinant polypeptide and the CAR are operably linked to the same promoter such that the two coding sequences are co-transcribed.
46. The population of embodiments 39 to 45, wherein the nucleic acid molecule is a viral vector, optionally a lentiviral or retroviral vector.
47. The population of any one of embodiments 32 to 46, wherein the populations of cells are human T cells.
48. The population of embodiment 47, wherein the populations of cells are human Treg cells.
49. A pharmaceutical composition comprising the population of embodiments 39 to 48, and a pharmaceutically acceptable carrier.

### Further Exemplary Embodiments

1. A recombinant polypeptide comprising an extracellular region, and a transmembrane region, wherein the extracellular region comprises some or all of an IL5 receptor alpha (IL5Ra) extracellular domain, and the extracellular region is linked to the transmembrane domain, wherein the recombinant polypeptide cannot function in signal transduction, and optionally wherein the recombinant polypeptide has decreased binding to IL-5.
2. The recombinant polypeptide of embodiment 1, wherein the recombinant polypeptide comprises an amino acid sequence having at least 80%, 85%, 90%, 95%, or 100% sequence identity with amino acids 32-123 (Domain I) of SEQ ID NO:1.
3. The recombinant polypeptide of embodiment 2, wherein the recombinant polypeptide further comprises an amino acid sequence having at least 80%, 85%, 90%, 95%, or 100% sequence identity with amino acids 124-242 (Domain II) of SEQ ID NO:1.
4. The recombinant polypeptide of embodiment 2 or embodiment 3, wherein the recombinant polypeptide further comprises an amino acid sequence having at least 80%, 85%, 90%, 95%, or 100% sequence identity with amino acids 243-334 (Domain III) of SEQ ID NO:1.
5. The recombinant polypeptide of embodiment 1, wherein the extracellular region comprises a truncated IL5Ra extracellular region comprising no more than some or all of:
i) the amino acid sequence of SEQ ID NO:2, SEQ ID NO: 60, SEQ ID NO:67, SEQ ID NO: 68 or SEQ ID NO:69;
ii) the amino acid sequence of SEQ ID NO: 61, SEQ ID NO: 70, SEQ ID NO: 71, or SEQ ID NO: 72; and/or
iii) the amino acid sequence of SEQ ID NO 73, SEQ ID NO 74, SEQ ID NO 75, SEQ ID NO 76, or SEQ ID NO 77.
6. The recombinant polypeptide of embodiment 1, wherein the extracellular region comprises a truncated IL5Ra extracellular region comprising some or all of the amino acid sequence of SEQ ID NO:2, SEQ ID NO: 60, SEQ ID NO:67, SEQ ID NO:68 or SEQ ID NO:69, but not the amino acid sequence of amino acids 124-342 of SEQ ID NO:1.
7. The recombinant polypeptide of any one of embodiments 1-6, wherein the extracellular region binds benralizumab.
8. The recombinant polypeptide of any one of embodiments 1-7, wherein the transmembrane region comprises an amino acid sequence having at least 80%, 85%, 90%, 95%, or 100% identity with amino acids 343-362 of SEQ ID NO:1.
9. The recombinant polypeptide of any one of embodiments 1-7, wherein the transmembrane region comprises an amino acid sequence having at least 80%, 85%, 90%, 95%, or 100% sequence identity with one amino acid sequence selected from the group consisting of SEQ ID NO:3, SEQ ID NO:4, SEQ ID NO:5, SEQ ID NO:6, SEQ ID NO:7, SEQ ID NO:8, SEQ ID NO: 9, SEQ ID NO:10, SEQ ID NO:11, SEQ ID NO:12, SEQ ID NO:78 and SEQ ID NO:79.
10. The recombinant polypeptide of any one of embodiments 1-9, wherein the recombinant polypeptide further comprises an intracellular domain.
11. The recombinant polypeptide of embodiment 10, wherein the intracellular domain comprises a truncated IL5Ra intracellular domain, optionally wherein the truncated IL5Ra intracellular domain does not associate with Janus kinase 2 (JAK2), optionally wherein the truncated IL5Ra intracellular domain has lost its signal transduction ability.
12. The recombinant polypeptide of embodiment 11, wherein
(i) the intracellular domain consists of amino acids 363-370 of SEQ ID NO:1; or
(ii) the intracellular domain comprises amino acids 363-366 of SEQ ID NO: 1 but not the amino acid sequence of SEQ ID NO:13.
13. The recombinant polypeptide of embodiment 10, wherein the intracellular domain is no longer than 25, 30, 35, 40, 45 or 50 amino acids in length and comprises the amino acid sequence of SEQ ID NO:14, SEQ ID NO:80, SEQ ID NO:81, SEQ ID NO:82, SEQ ID NO:83, SEQ ID NO:84 or SEQ ID NO:85.
14. The recombinant polypeptide of embodiment 1, wherein the extracellular domain comprises an amino acid sequence having at least 80%, 85%, 90%, 95%, or 100% sequence identity with amino acids 32-123 (Domain I) or amino acids 32-242 (Domains I and II) or amino acids 32-334 (Domains I, II and III) of SEQ ID NO:1.
15. The recombinant polypeptide of embodiment 1, wherein the recombinant polypeptide comprises an amino acid sequence having at least 80%, 85%, 90%, 95%, or 100% sequence identity with amino acids 32-370 (Domains I, II and III, transmembrane domain and a fragment of the intracellular domain) of SEQ ID NO:1.
16. The recombinant polypeptide of embodiment 1, wherein the recombinant polypeptide comprises an amino acid sequence having at least 95% sequence identity with amino acids 32 to 123, or 32 to 242, or 32 to 334, or 32 to 342, or 32 to 370 of SEQ ID NO:1.
17. The recombinant polypeptide of any one of embodiments 1-16, further comprising a signal peptide.
18. The recombinant polypeptide of embodiment 17, wherein the signal peptide comprises an amino acid sequence having at least 80%, 85%, 90%, 95%, or 100% sequence identity with the amino acid sequence of SEQ ID NO:58, SEQ ID NO:15, SEQ ID NO:63, SEQ ID NO: 64, SEQ ID NO:65, or SEQ ID NO:66.
19. The recombinant polypeptide of one of embodiments 1-18, further comprising a linker between the extracellular domain and the transmembrane domain.
20. The recombinant polypeptide of embodiment 19, wherein the linker comprises an amino acid sequence having at least 80%, 85%, 90%, 95%, or 100% sequence identity with the amino acid sequence of SEQ ID NO:16, SEQ ID NO: 17, SEQ ID NO:18, SEQ ID NO: 19, SEQ ID NO: 20, SEQ ID NO:21 and SEQ ID NO:22.
21. The recombinant polypeptide of embodiment 10, wherein the intracellular domain consists of the amino acid sequence of SEQ ID NO: 14, SEQ ID NO:80, SEQ ID NO:81, SEQ ID NO: 82, SEQ ID NO:83, SEQ ID NO:84, SEQ ID NO:85; or an amino acid sequence at least 80%, 85%, 90%, or 95% identical thereto.
22. The recombinant polypeptide of embodiment 1, wherein the recombinant polypeptide comprises the amino acid sequence:
i) selected from the group consisting of SEQ ID NOs: 23-40, or an amino acid sequence at least 80%, 85%, 90%, or 95% identical thereto; or
(ii) selected from the group consisting of SEQ ID NOs: 148-208, or an amino acid sequence at least 80%, 85%, 90%, or 95% identical thereto; or
(iii) selected from the group consisting of SEQ ID NOs: 23-40 or from the group consisting of 148-208, or an amino acid sequence at least 80%, 85%, 90%, or 95% identical thereto.
23. The recombinant polypeptide of embodiment 1, comprising a heterologous region of at least 4 amino acids in length, wherein the amino acid sequence of the heterologous region is heterologous to IL5Ra, optionally wherein the amino acid sequence of the heterologous region is not present in the amino acid sequence of human IL5Ra of SEQ ID NO:1. That is, the amino acid sequence of the heterologous region is not 100% identical to fragment of human IL5 Ra of SEQ ID NO: 1.
24. A nucleic acid molecule comprising a coding sequence for the recombinant polypeptide of any one of the preceding embodiments.
25. The nucleic acid molecule of embodiment 24, wherein the nucleic acid molecule comprises the nucleotide sequence:
(i) selected from the group consisting of SEQ ID NOs: 41-57 and 86, or a nucleotide sequence at least 80%, 85%, 90%, 95% or 100% identical thereto; or
(ii) selected from the group consisting of SEQ ID NOs: 87-147, or a nucleotide sequence at least 80%, 85%, 90%, 95% or 100% identical thereto; or
(iii) selected from the group consisting of SEQ ID NOs: 41-57 and 86, or from the group consisting of SEQ ID NOs: 87-147, or an nucleotide sequence at least 80%, 85%, 90%, 95% or 100% identical thereto.
26. The nucleic acid molecule of embodiment 24, further comprising a coding sequence for a chimeric antigen receptor (CAR).
27. The nucleic acid molecule of embodiment 26, wherein the coding sequences for the recombinant polypeptide and the CAR are operably linked to the same promoter such that the two coding sequences are co-transcribed.
28. The nucleic acid molecule of any one of embodiments 24-27, wherein the nucleic acid molecule is present in a viral vector, optionally wherein the viral vector is a lentiviral vector or a retroviral vector.
29. A cell comprising the nucleic acid molecule of any one of embodiments 24-28.
30. The cell of embodiment 29, wherein the cell is a human T cell.
31. The cell of embodiment 30, wherein the cell is a human Treg cell.
32. A pharmaceutical composition comprising: (i) the cell of any one of embodiments 29-31, or the nucleic acid molecule of any one of embodiments 21-28; and (ii) a pharmaceutically acceptable carrier.
33. A method of treating a patient in need thereof, comprising administering the cell of any one of embodiments 29 to 31 to the patient, optionally wherein the cell is derived from the patient.
34. A method of treating a patient in need thereof, comprising administering the cell of embodiment 30 or embodiment 31 to the patient, wherein the cell expresses a CAR specific for an antigen present in a disease the patient is suffering from.
35. The method of embodiment 33 or embodiment 34, further comprising administering to the patient an effective amount of an antibody specific for IL5Ra once the patient has been treated, wherein the antibody elicits cytotoxicity against cells expressing the recombinant polypeptide, optionally wherein the antibody is IgG1 or IgG2.
36. A method of making a genetically engineered human cell, comprising providing an isolated human cell, and introducing the nucleic acid molecule of any one of embodiments 24-28 into the human cell.
37. The method of embodiment 36, wherein the human cell is a human T cell.
38. The method of embodiment 37, wherein the human cell is a human Treg cell.
39. The method of any one of embodiments 36-38, further comprising culturing the human cell under conditions for expression of the recombinant polypeptide on the surface of the genetically engineered human cell.
40. A pharmaceutical composition comprising: (i) a plurality of the cells (e.g., a population of cells) of any one of embodiments 29-31, and (ii) a pharmaceutically acceptable carrier.
41. A pharmaceutical composition comprising: (i) a plurality of the genetically engineered human cells (e.g., a population of cells) produced by the method of any one of embodiments 36-39, and (ii) a pharmaceutically acceptable carrier.
42. The recombinant polypeptide of any one of embodiments 1-23, wherein the recombinant polypeptide has decreased binding to IL-5.

### EXAMPLES

Abbreviations: ADCC (antibody-dependent cell-mediated cytotoxicity); CAR (chimeric antigen receptor); CV (citrullinated vimentin); FI (fluorescence intensity); (IL-5Ra (interleukin-5 receptor alpha); IL-5Rat (IL-5Ra truncated); mAb (monoclonal antibody); MFI (median fluorescence intensity).

### Example 1: Expression of Surface Markers in Primary Treg Cells

Treg cells were purified from PBMCs. CD4+ were enriched by negative selection from PBMCs by magnetic cell sorting (Miltenyi Biotec). CD4 T cells were then stained with fluorochrome-labelled mAb specific for CD4, CD25 and CD127 and sorted by flow cytometry into CD4+CD25high CD127low cells. Purified primary Tregs were expanded via anti-CD3 and anti-CD28 coated Dynabeads at a ratio 1:2 in the presence of IL-2 (300 U/ml) in T cell media, RPMI with 10% FBS. Fresh media containing IL-2 was added every 2 days and cells were split when needed.

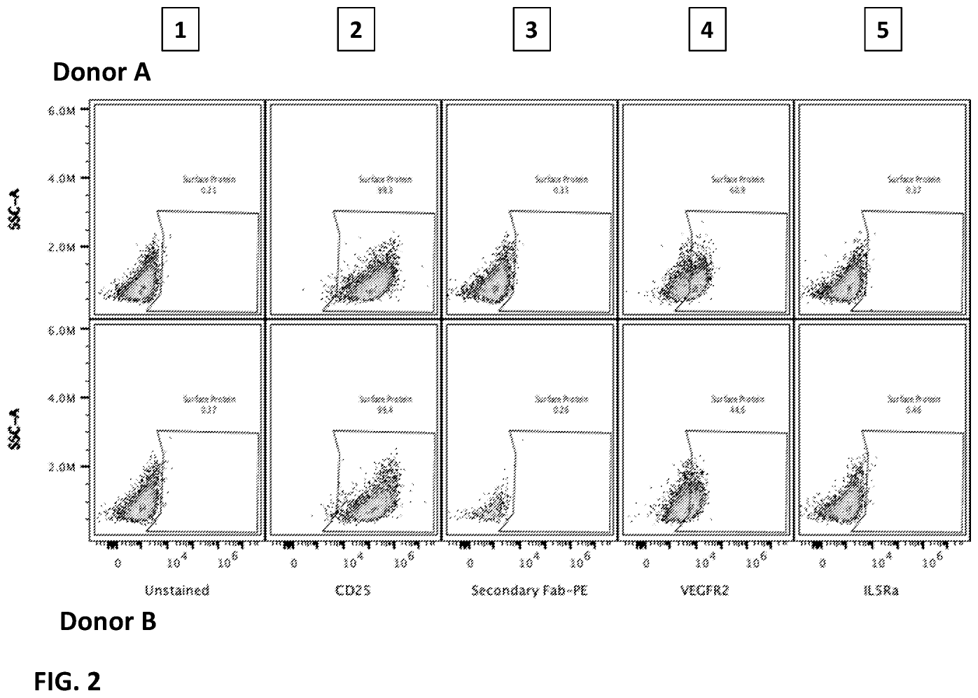

On day 14 of expansion, the cells were stained for the expression of surface proteins. Two different donors were used. Donor B received Fc block during the staining protocol whereas Donor A did not. After staining these cells were run on a flow cytometer. The anti-Human VEGF Receptor 2 Therapeutic Antibody (Ramucirumab) and anti-Human IL5RA Therapeutic Antibody (Benralizumab) were purchased from Creative Biolabs. Each of these antibodies were used to stain the primary Tregs followed by washing and staining with a secondary anti-human Fab-PE antibody. The results are shown in FIG. 2.

FIG. 2, dot plots (1) show unstained primary Tregs. FIG. 2 dot plots (2) show a positive control where the primary Tregs were stained with an anti-CD25-PE antibody. As expected, the purified Treg cells were positive for CD25. FIG. 2 dot plots (3) show the cells that were stained with only the secondary anti-human Fab-PE antibody. No background staining was detected. FIG. 2 dot plots (4) and (5) show staining with an anti-Human VEGF Receptor 2 Therapeutic Antibody (Ramucirumab) and anti-Human IL5RA Therapeutic Antibody (Benralizumab), respectively. The results demonstrate that Ramucirumab can bind to VEGF Receptor 2 which is on the surface of activated Tregs and that Benralizumab cannot bind to activated Tregs because of their lack of expression of IL5Ra on the Treg cell surface. Thus, this makes IL5Ra orthogonal to activated Tregs.

### Example 2: Expression of Cellular Tags in Cells

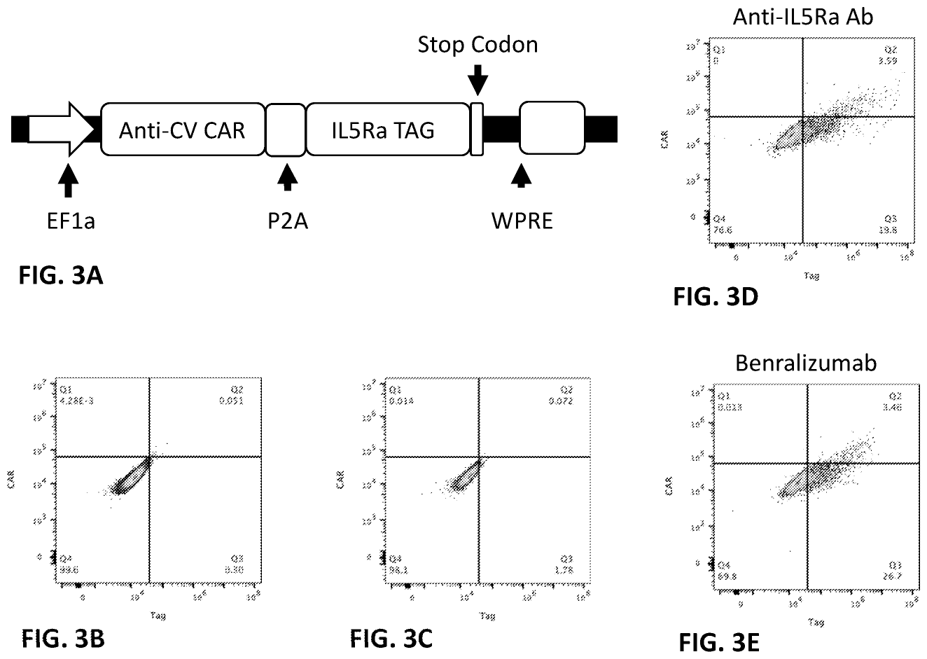

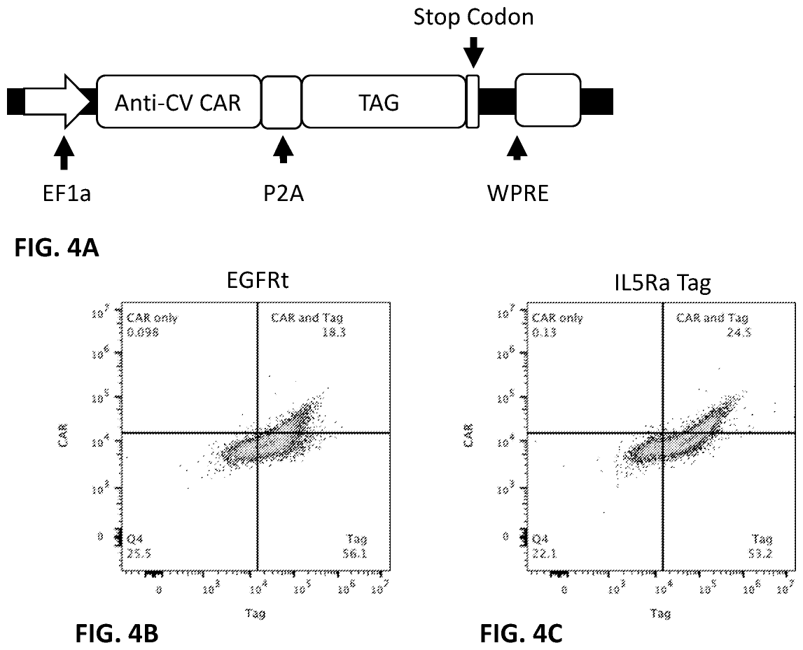

In order to test the expression of the IL5Rat cellular tag, a plasmid encoding a IL5Rat cellular tag was first transiently transfected into K562 cells. Upon the success of the transient transfection the plasmid was made into lentivirus where it was transduced into Jurkat cells. FIG. 3A and FIG. 4A depict diagrams of the expression cassette. The promoter EF1a is followed by an anti-Citrullinated Vimentin (CV) chimeric antigen receptor (CAR). This is followed by a ribosomal skip sequence, P2A, the tag and the stop codon. The end of the cassette is followed by WPRE. In these experiments cell cultures were transduced with the plasmid pSB_0166, a plasmid backbone designed for 3rd generation lentivirus. The IL5Rat tag in the pSB_0166 plasmid contains the full extracellular domain of IL5Rat to the transmembrane domain to the first 4 amino acids of the intracellular domain:

(SEQ ID NO: 27)
MLLLVTSLLLCELPHPAFLLIPDLLPDEKISLLPPVNFTIKVTGLAQVL
LQWKPNPDQEQRNVNLEYQVKINAPKEDDYETRITESKCVTILHKGESA
SVRTILQNDHSLLASSWASAELHAPPGSPGTSIVNLTCTTNTTEDNYSR
LRSYQVSLHCTWLVGTDAPEDTQYFLYYRYGSWTEECQEYSKDTLGRNI
ACWFPRTFILSKGRDWLAVLVNGSSKHSAIRPFDQLFALHAIDQINPPL
NVTAEIEGTRLSIQWEKPVSAFPIHCFDYEVKIHNTRNGYLQIEKLMTN
AFISIIDDLSKYDVQVRAAVSSMCREAGLWSEWSQPIYVGNDEHKPLRE
WFVIVIMATICFILLILSLICKICH.

The plasmid was transiently transfected into cell lines via electroporation. The Amaxa® 4D-Nucleofector® protocol was used for transient transfection following the protocol from Lonza. Lentivirus was made and titered. The titered virus was stained for both CAR expression via protein L staining and the presence of tag by an antiIL5Ra-PE antibody (data no shown). pSB_0166 can be made into high grade lentivirus.

After 48 hours the cells were stained and run on the flow cytometer using a CV peptide conjugated with biotin that has been bound to streptavidin-FITC for CAR detection and either a commercial antiIL5Ra-PE or Benralizumab (Creative Biolab) followed by a secondary anti-human Fab-PE for IL5Ra detection, in this case the IL5Rat tags.

FIG. 3A shows the expression cassette of the IL5Rat tag in plasmid pSB_0166, which was used to transiently transfect K562 cells. FIG. 3B shows dot plots of K562 control cells that have not been transiently transfected after staining with the full antibody cocktail. FIG. 3C show dot plots of transfected cells that were stained with a CV-biotin that has been bound to streptavidin-FITC for CAR detection and an anti-human Fab-PE antibody, which demonstrates that no staining of the CAR is detected from the anti-human Fab-PE antibody. FIG. 3D-3E show dot plots of transfected cells that were stained with a CV-biotin that has been bound to streptavidin-FITC for CAR detection and either a commercial anti-IL5Ra-PE or Benralizumab (Creative Biolab) followed by a secondary anti-human Fab-PE. pSB_0166 work equally as well with each staining routine. Thus, the IL5Rat tag can be used as a cellular marker to detect transfected cells. FIG. 4A shows the expression cassettes for expression cell surface tags. FIG. 4B shows expression of an EGFRt tag and FIG. 4C shows expression of an IL5Rat tag in transduced Jurkat cells. The IL5Rat tag was expressed upon transduction with a lentivirus produced from the pSB_0166 plasmid, while the EGFRt tag was expressed upon transduction with a lentivirus produced from a plasmid identical to pSB_0166 except that it harbors the coding region of the conical EGFRt tag instead of the coding region of the IL5Rat tag. Jurkat cells were transduced at an MOI of 3 with each of the viruses. On day 3 post transduction, each of the transduced cells were stained for the presence of the anti-CV CAR and the tag. The dot plots of FIG. 4B and FIG. 4C show that the IL5Rat tag express comparably well to EGFRt tag.

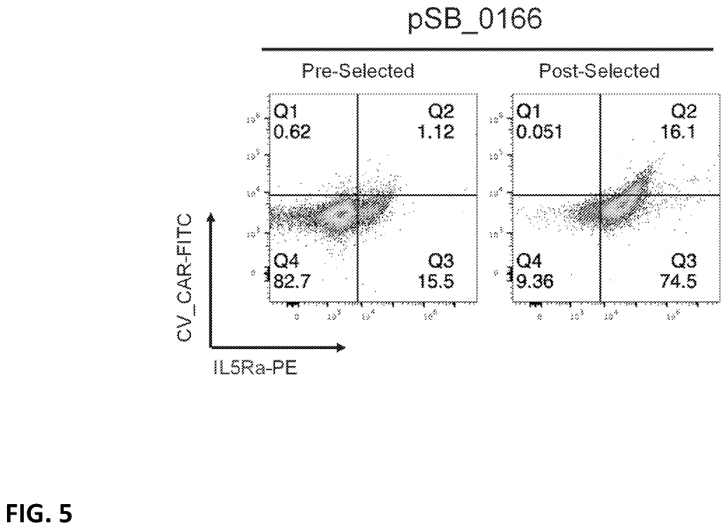

To determine whether cells can be positively selected with the IL5Rat tag, Jurkat cells were transduced at an MOI of 0.5 with viruses made from pSB_0166. On day 7 post transduction, each of the transduced cells were placed through a positive selection process. The IL5Rat tag was stained with an anti-CD125-PE antibody and then anti-PE MicroBeads (Miltenyi Biotec) were applied. The stained cells were placed on an LS column (Miltenyi Biotech) for positive selection. Two days post positive selection cultures of pre-selected and post-selected cells were stained for the CAR and the IL5Rat tag and run-on a flow cytometer. FIG. 5 shows that the IL5Rat tag can be positively selected and yield a pure population of cells expressing the IL5Rat tag.

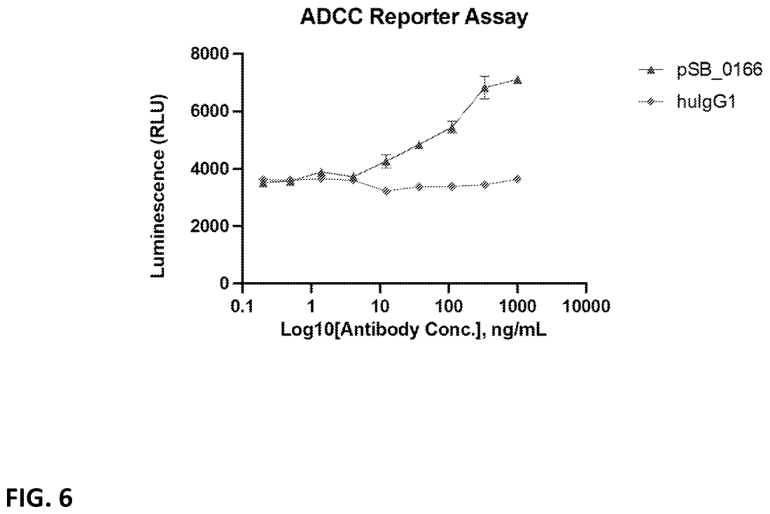

Ablation of cells expressing the IL5Rat tag will happen by binding of benralizumab to the tag exposed on the cell surface leading to Fc receptor-mediated antibody-dependent cell-mediated cytotoxicity (ADCC). To test this feature, target Jurkat cells expressing the IL5Rat tag (pSB_166) were placed into a ADCC reporter assay. The ADCC reporter assay measures FcgR engagement which correlates to ADCC capabilities. ADCC reporter assay uses a stable effector Jurkat cell line expressing human FcgRIIIa V158 and NFAT-induced luciferase. The effector to target ratio was 1:1 with variable amounts of Benralizumab or human IgG1 (negative control). The cells were incubated overnight before Bio-Glo™ (Promega) was added to activate the luciferase present in the effector cells. Luminescence was measured on a Molecular Devices SpectraMax iD3 plate reader. FIG. 6 shows that IL5Rat tag expression upon transduction with a lentivirus produced from pSB_0166 activates the effector cells in a dose dependent way, which indicated that the IL5Rat tag is a suitable target for ablation via ADCC.

### Example 3: Expression of IL5Rat Tags in Cells

Different combinations of transmembrane domains and intracellular domains were tested by transduction of Jurkat cells with lentivirus comprising a bicistronic expression cassette for expression of a CV-CAR and an IL5Rat tag. The methods employed in this example were as described in Example 2 above. The amino acid sequences of exemplary IL5Rat tags and the nucleotide sequences encoding the exemplary IL5Rat tags are set forth in the SEQ ID NOs. of Table 3-1. In the table below, ID refers to the plasmid name, SEQ-N refers to the SEQ ID NO for the nucleotide sequence, SEQ-P refers to the SEQ ID NO for the amino acid sequence, and AA refers to the length of the IL5RAt tag.

TABLE 3-1
Exemplary IL5Rat tag Sequences

| ID | SEQ-N | Description | SEQ-P | AA |
|---|---|---|---|---|
| pSB_0194 | 87 | IL5Rat(TM1) | 148 | 363 |
| pSB_0195 | 88 | IL5Rat(TM2) | 149 | 365 |
| pSB_0196 | 89 | IL5Rat(S1) | 150 | 373 |
| pSB_0197 | 90 | IL5Rat(S2(C367G)) | 151 | 373 |
| pSB_0198 | 91 | IL5Rat(S3) | 152 | 369 |
| pSB_0322 | 92 | IL5Rat(S4(C367G)) | 153 | 369 |
| pSB_0323 | 93 | IL5Rat(S5) | 154 | 371 |
| pSB_0324 | 94 | IL5Rat(S6(C367G)) | 155 | 371 |
| pSB_0325 | 95 | IL5Rat(S7-KRR) | 156 | 368 |
| pSB_0326 | 96 | IL5Rat(S8-WFLKRE) | 157 | 371 |
| pSB_0327 | 97 | IL5Rat(I69A) | 158 | 369 |
| pSB_0328 | 98 | IL5Rat(S64A) | 159 | 369 |
| pSB_0330 | 99 | IL5Rat(R188A) | 160 | 369 |
| pSB_0331 | 100 | IL5Rat(I69A, S64A) | 161 | 369 |
| pSB_0332 | 101 | IL5Rat(I69A, I183A) | 162 | 369 |
| pSB_0333 | 102 | IL5Rat(I69A, R188A) | 163 | 369 |
| pSB_0334 | 103 | IL5Rat(I183A, R188A) | 164 | 369 |
| pSB_0335 | 104 | IL5Rat(I69A, I183A, R188A) | 165 | 369 |
| pSB_0336 | 105 | IL5Rat(I69A, S64A, I183A, R188A) | 166 | 369 |
| pSB_0354 | 106 | IL5Rat(I69A, I183A, R188A) | 167 | 369 |
| pSB_0357 | 107 | IL5Rat(S9-KRRQQK) | 168 | 371 |
| pSB_0512 | 108 | IL5Ra(I69A) + (IC_Full) | 169 | 423 |
| pSB_0514 | 109 | IL5Rat(S_CD19_1) | 170 | 384 |
| pSB_0515 | 110 | IL5Rat(S_CD19_2) | 171 | 377 |
| pSB_0516 | 111 | IL5Rat(S_CD19_3) | 172 | 374 |
| pSB_0539 | 112 | IL5Rat(I183A) | 173 | 369 |
| pSB_0540 | 113 | IL5Rat(K186A) | 174 | 369 |
| pSB_0541 | 114 | IL5Rat(R188A) | 175 | 369 |
| pSB_0542 | 115 | IL5Rat(M295A) | 176 | 369 |
| pSB_0543 | 116 | IL5Rat(I183A, K186A) | 177 | 369 |
| pSB_0544 | 117 | IL5Rat(I183A, R188A) | 178 | 369 |
| pSB_0545 | 118 | IL5Rat(I183A, M295A) | 179 | 369 |
| pSB_0546 | 119 | IL5Rat(K186A, R188A) | 180 | 369 |
| pSB_0547 | 120 | IL5Rat(K186A, M295A) | 181 | 369 |
| pSB_0548 | 121 | IL5Rat(R188A, M295A) | 182 | 369 |
| pSB_0549 | 122 | IL5Rat(I183A, K186A, R188A) | 183 | 369 |
| pSB_0550 | 123 | IL5Rat(I183A, K186A, M295A) | 184 | 369 |
| pSB_0551 | 124 | IL5Rat(I183A, R188A, M295A) | 185 | 369 |
| pSB_0552 | 125 | IL5Ra(K186A, R188A, M295A) | 186 | 369 |
| pSB_0553 | 126 | IL5Rat(I183A, K186A, R188A, M295A) | 187 | 369 |
| pSB_0554 | 127 | IL5Rat(S64A) | 188 | 369 |
| pSB_0590 | 128 | IL5RatEC_Her2(TMIC)(S1) | 189 | 374 |
| pSB_0591 | 129 | IL5RatEC_Her2(TMIC)(S2) | 190 | 371 |
| pSB_0592 | 130 | IL5RatEC_CD19(TMIC)(S1) | 191 | 386 |
| pSB_0593 | 131 | IL5RatEC_CD19(TMIC)(S2) | 192 | 379 |
| pSB_0594 | 132 | IL5RatEC_PDGFR(TMIC)(S1) | 193 | 369 |
| pSB_0595 | 133 | IL5RatEC_PDGFR(TMIC)(S2) | 194 | 377 |
| pSB_0596 | 134 | IL5RatEC_PDGFR(TMIC)(S3) | 195 | 383 |
| pSB_0597 | 135 | IL5RatEC_EPH(TMIC)(S1) | 196 | 373 |
| pSB_0598 | 136 | IL5RatEC_EPH(TMIC)(S2) | 197 | 378 |
| pSB_0599 | 137 | IL5RatEC_EPH(TMIC)(S3) | 198 | 385 |
| pSB_0679 | 138 | IL5Rat(K186A, R188A)EC_Her2(TMIC)(S1) | 199 | 374 |
| pSB_0680 | 139 | IL5Rat(K186A, R188A, M295A)EC_Her2(TMIC)(S1) | 200 | 374 |
| pSB_0684 | 140 | IL5Ra(R188A)(fullIC) | 201 | 423 |
| pSB_0685 | 141 | IL5Ra((K186A, R188A)(fullIC) | 202 | 423 |
| pSB_0686 | 142 | IL5Ra((K186A, R188A, M295A)(fullIC) | 203 | 423 |
| pSB_0687 | 143 | IL5Rat(C296A) | 204 | 369 |
| pSB_0688 | 144 | IL5Rat(R297A) | 205 | 369 |
| pSB_0689 | 145 | IL5Rat(E298A) | 206 | 369 |
| pSB_0690 | 146 | IL5Rat(R188G) | 207 | 369 |
| pSB_0693 | 147 | IL5Rat(K186A)EC_Her2(TMIC)(S1) | 208 | 374 |

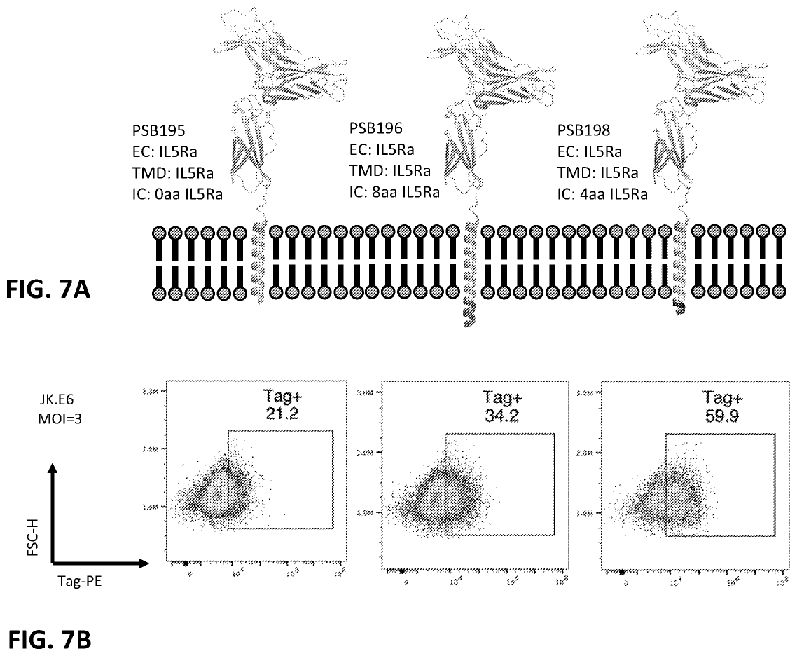

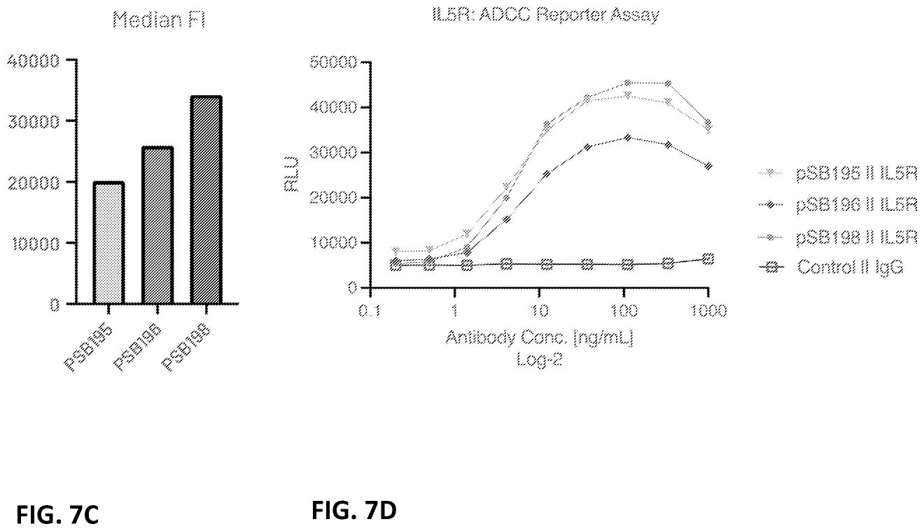

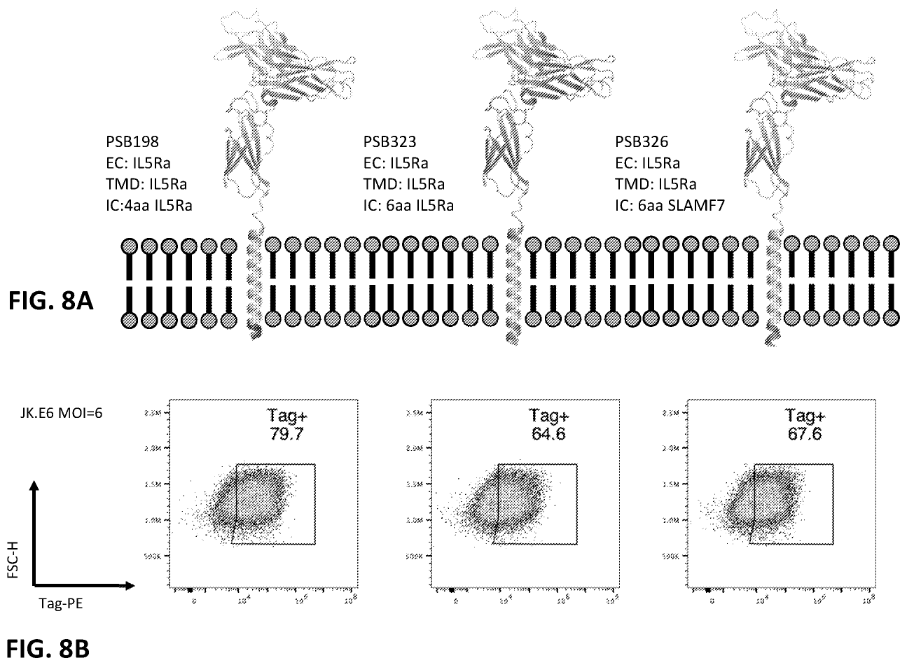

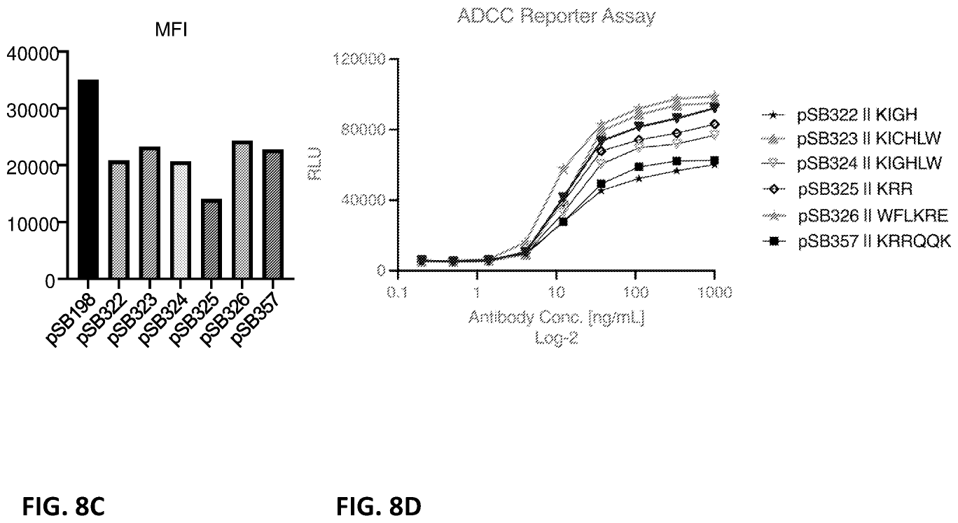

FIG. 7A and FIG. 8A illustrate the structures of several different IL5Rat tags. FIG. 7B and FIG. 8B show percentages of transduced Jurkat cells expressing different IL5Rat tags. FIG. 7C and FIG. 8C show levels of expression (median fluorescence intensity) of different IL5Rat tags on transduced Jurkat cells.

FIG. 7D and FIG. 8D show differences in ablatability of transduced Jurkat cells expressing different IL5Rat tags. In this experiment, ablatability was measured in an ADCC reporter assay employing varying amounts of benralizumab.

These results demonstrate that engineering the transmembrane domain and the intracellular domain of an IL5Rat tag can improve functional properties of this type of cell surface tag.

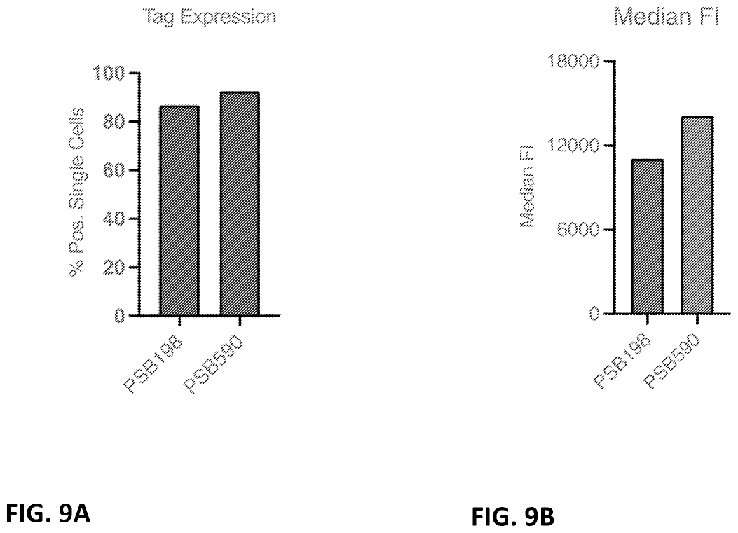

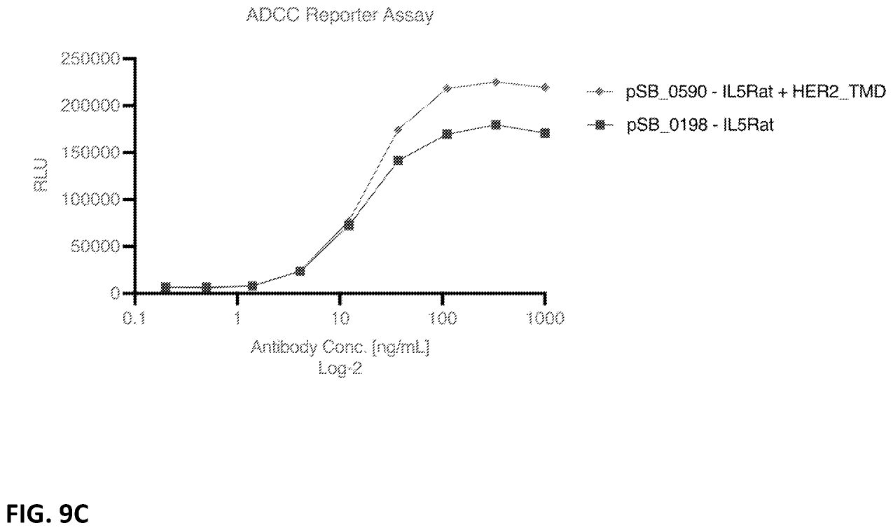

FIG. 9A and FIG. 9B shows percentages of transduced Jurkat cells expressing IL5Rat tags (pSB198 or pSB590), and levels of expression of the IL5Rat tags on transduced Jurkat cells. FIG. 9C shows differences in the ability of benralizumab to bring about ADCC of transduced Jurkat cells expressing the IL5Rat tags. In brief, these results indicate that inclusion of a chimeric HER2 transmembrane domain in the IL5Rat tags results in enhanced expression and benralizumab-mediated ADCC.

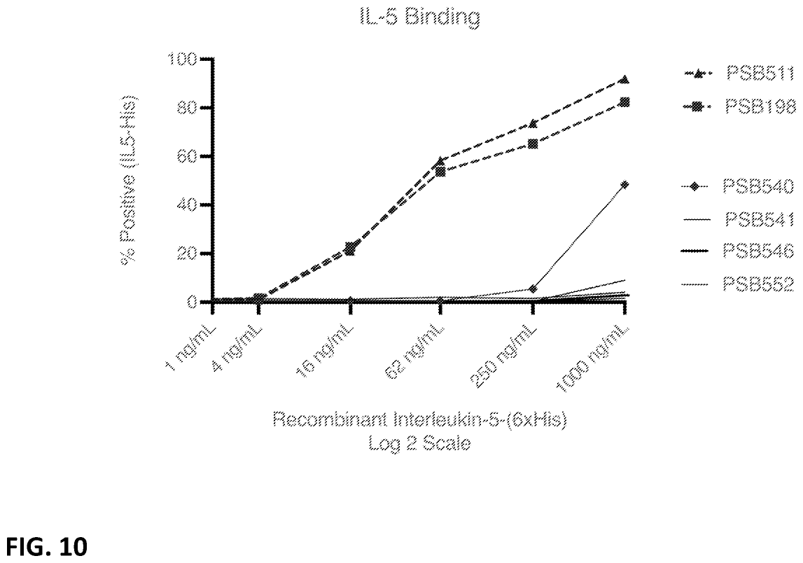

FIG. 10 shows binding of recombinant IL-5 to transduced Jurkat cells expressing various IL5Rat tags. These results indicate that transduced cells expressing IL5Rat tags with mutant extracellular domains (pSB540, pSB541, pSB546, or pSB552) exhibit diminished IL-5 binding relative to transduced cells expressing IL5Rat tags with wild-type extracellular domains (pSB511 or pSB198).

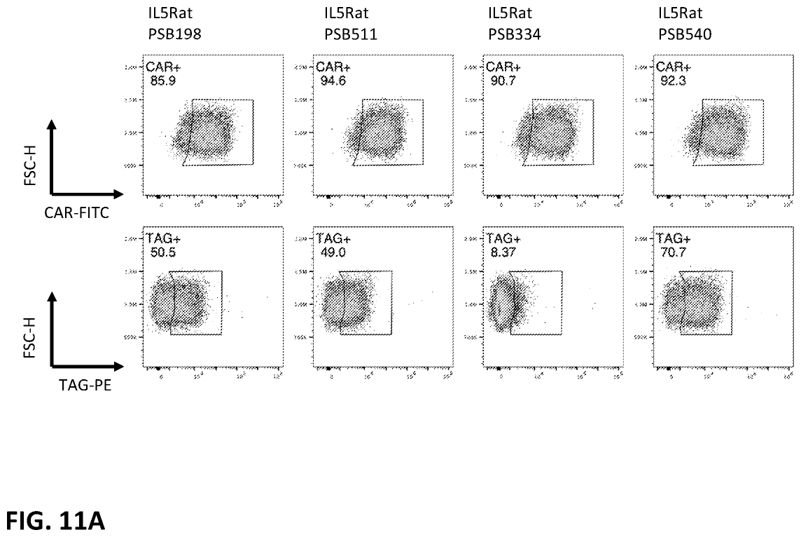

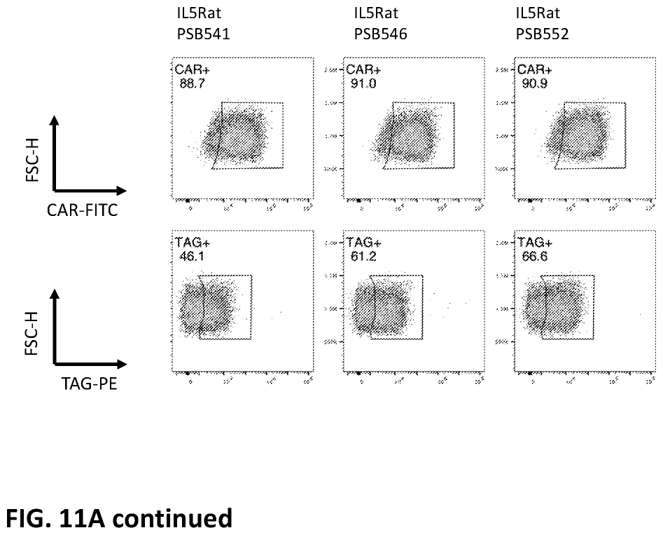

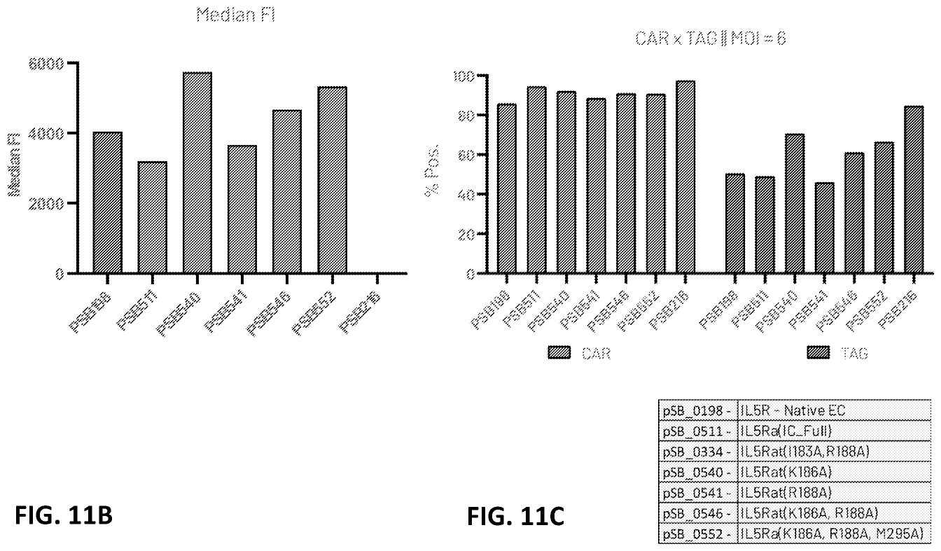

FIG. 11A-11C show that transduced cells retain cell surface expression of IL5Rat tags with mutant extracellular domains, despite reductions in IL-5 binding.

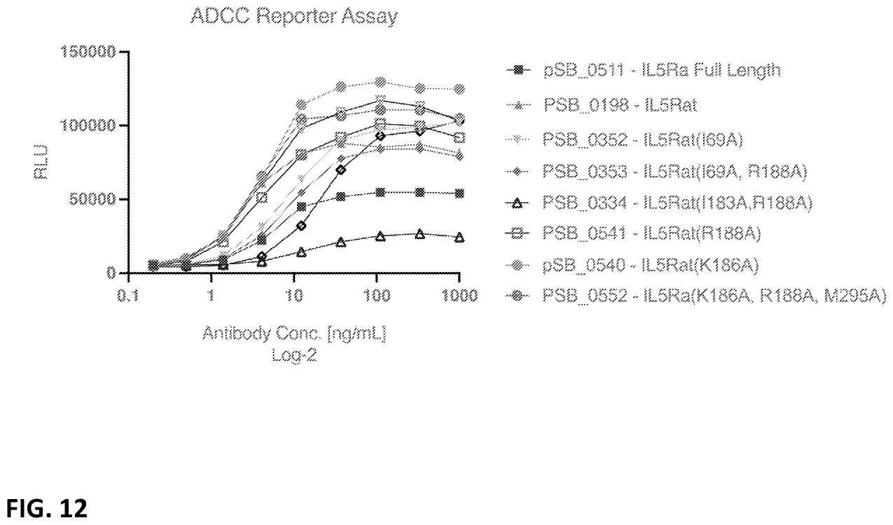

FIG. 12 shows differences in the ability of benralizumab to bring about ADCC of transduced Jurkat cells expressing IL5Rat tags with mutant extracellular domains.

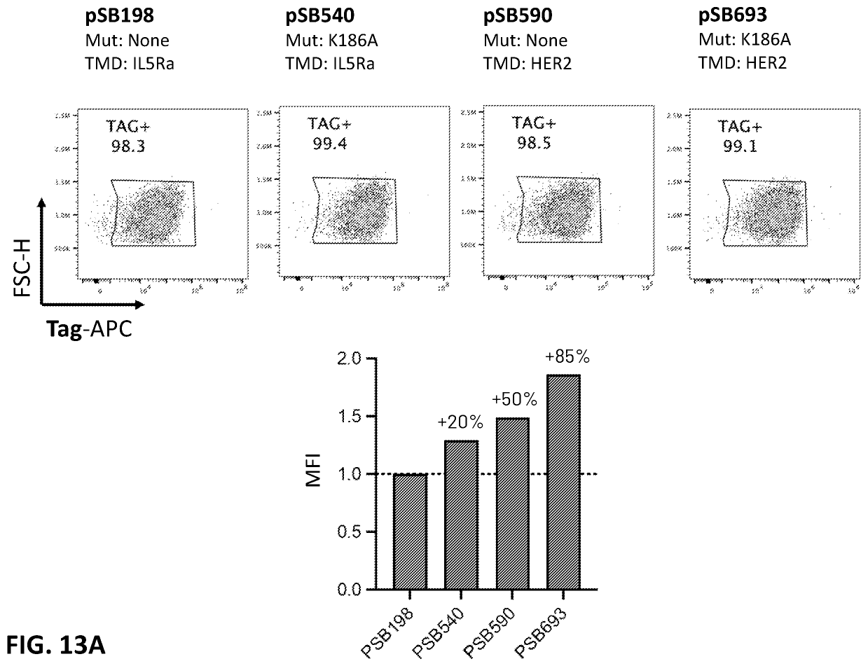

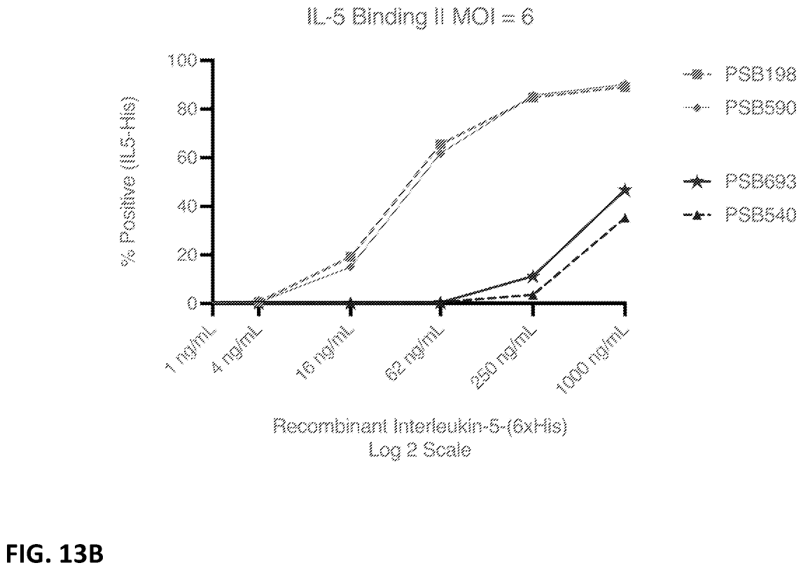

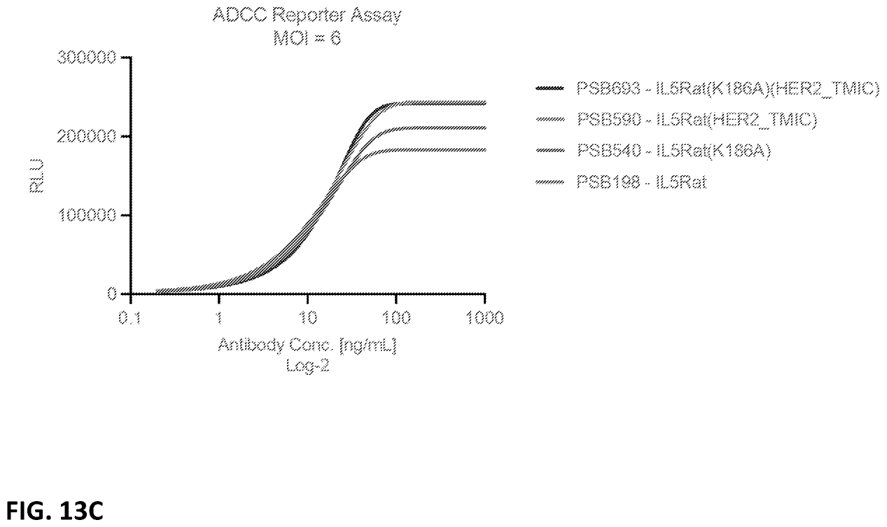

FIG. 13A shows percentages of transduced Jurkat cells expressing different IL5Rat tags, and levels of expression (median fluorescence intensity) of different IL5Rat tags on transduced Jurkat cells. These results indicate that transduced cells expressing either an IL5Rat tag with a mutant extracellular domain (pSB540) and/or a heterologous transmembrane domain (pSB590 and pSB693) have higher levels of IL5Rat tag expression than transduced cells expressing a comparator IL5Rat tag with a wild-type transmembrane domain and a homologous transmembrane domain (pSB198).

FIG. 13B shows binding of recombinant IL-5 to transduced Jurkat cells expressing various IL5Rat tags. These results indicate that transduced cells expressing IL5Rat tags with mutant extracellular domains (pSB540, or pSB693) exhibit diminished IL-5 binding relative to transduced cells expressing IL5Rat tags with wild-type extracellular domains (pSB198 or pSB590).

FIG. 13C shows differences in the ability of benralizumab to bring about ADCC of transduced Jurkat cells expressing various IL5Rat tags.

### Example 4: Expression of a CAR and a Cell Surface Tag in Primary Treg Cells

In this example, sustained expression of a CAR and a cell surface tag (TAG) was assessed in transduced human Treg cells that were subjected to a 14-day expansion protocol. The methods employed in this example were as described in Example 1 above.

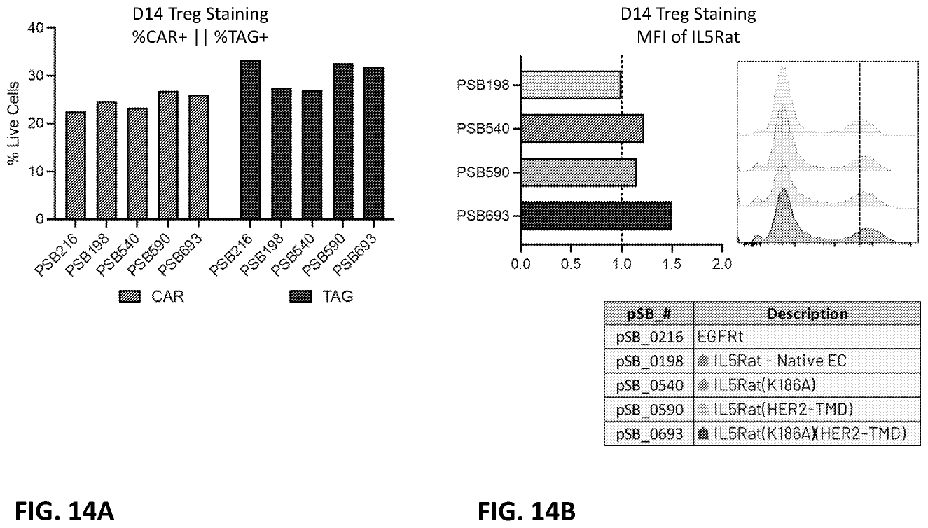

FIG. 14A and FIG. 14B show that CAR and TAG expression is maintained in expanded Treg populations.

It should be understood that the description and the drawings are not intended to limit the invention to the particular form disclosed, but to the contrary, the intention is to cover all modifications, equivalents, and embodiments falling within the spirit and scope of the present invention as defined by the appended claims. Further modifications and alternative embodiments of various aspects of the invention will be apparent to those skilled in the art in view of this description. Accordingly, this description and the drawings are to be construed as illustrative only and are for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed or omitted, and certain features of the invention may be utilized independently, all as would be apparent to one skilled in the art after having the benefit of this description of the invention. Changes may be made in the elements described herein without departing from the spirit and scope of the invention as described in the following claims. Headings used herein are for organizational purposes only and are not meant to be used to limit the scope of the description.

All publications, patents, and patent applications mentioned in this specification are herein incorporated by reference to the same extent as if each individual publication, patent, or patent application was specifically and individually indicated to be incorporated by reference.

We claim:

1. A recombinant polypeptide comprising an extracellular region, and a transmembrane region, wherein the extracellular region comprises some or all of an IL5 receptor alpha (IL5Ra) extracellular domain, and the extracellular region is linked to the transmembrane domain, wherein the recombinant polypeptide cannot function in signal transduction, and optionally wherein the recombinant polypeptide has decreased binding to IL-5.

2. The recombinant polypeptide of claim 1, wherein the recombinant polypeptide comprises an amino acid sequence having at least 80%, 85%, 90%, 95%, or 100% sequence identity with amino acids 32-123 (Domain I) of SEQ ID NO:1.

3. The recombinant polypeptide of claim 2, wherein the recombinant polypeptide further comprises an amino acid sequence having at least 80%, 85%, 90%, 95%, or 100% sequence identity with amino acids 124-242 (Domain II) of SEQ ID NO:1.

4. The recombinant polypeptide of claim 2, wherein the recombinant polypeptide further comprises an amino acid sequence having at least 80%, 85%, 90%, 95%, or 100% sequence identity with amino acids 243-334 (Domain III) of SEQ ID NO:1.

5. The recombinant polypeptide of claim 1, wherein the extracellular region comprises a truncated IL5Ra extracellular region comprising no more than some or all of:
i) the amino acid sequence of SEQ ID NO:2, SEQ ID NO: 60, SEQ ID NO:67, SEQ ID NO: 68 or SEQ ID NO:69;
ii) the amino acid sequence of SEQ ID NO: 61, SEQ ID NO: 70, SEQ ID NO: 71, or SEQ ID NO: 72; and/or
iii) the amino acid sequence of SEQ ID NO 73, SEQ ID NO 74, SEQ ID NO 75, SEQ ID NO 76, or SEQ ID NO 77.

6. The recombinant polypeptide of claim 1, wherein the extracellular region comprises a truncated IL5Ra extracellular region comprising some or all of the amino acid sequence of SEQ ID NO:2, SEQ ID NO: 60, SEQ ID NO:67, SEQ ID NO:68 or SEQ ID NO:69, but not the amino acid sequence of amino acids 124-342 of SEQ ID NO:1.

7. The recombinant polypeptide of claim 1, wherein the extracellular region binds benralizumab.

8. The recombinant polypeptide of claim 1, wherein the transmembrane region comprises an amino acid sequence having at least 80%, 85%, 90%, 95%, or 100% identity with amino acids 343-362 of SEQ ID NO:1.

9. The recombinant polypeptide of claim 1, wherein the transmembrane region comprises an amino acid sequence having at least 80%, 85%, 90%, 95%, or 100% sequence identity with one amino acid sequence selected from the group consisting of SEQ ID NO:3, SEQ ID NO: 4, SEQ ID NO:5, SEQ ID NO:6, SEQ ID NO:7, SEQ ID NO:8, SEQ ID NO:9, SEQ ID NO: 10, SEQ ID NO:11, SEQ ID NO:12, SEQ ID NO:78 and SEQ ID NO:79.

10. The recombinant polypeptide of claim 1, wherein the recombinant polypeptide further comprises an intracellular domain.

11. The recombinant polypeptide of claim 10, wherein the intracellular domain comprises a truncated IL5Ra intracellular domain, optionally wherein the truncated IL5Ra intracellular domain does not associate with Janus kinase 2 (JAK2), optionally wherein the truncated IL5Ra intracellular domain has lost its signal transduction ability.

12. The recombinant polypeptide of claim 11, wherein
(i) the intracellular domain consists of amino acids 363-370 of SEQ ID NO:1; or
(ii) the intracellular domain comprises amino acids 363-366 of SEQ ID NO: 1 but not the amino acid sequence of SEQ ID NO:13.

13. The recombinant polypeptide of claim 10, wherein the intracellular domain is no longer than 25, 30, 35, 40, 45 or 50 amino acids in length and comprises the amino acid sequence of SEQ ID NO:14, SEQ ID NO:80, SEQ ID NO:81, SEQ ID NO: 82, SEQ ID NO:83, SEQ ID NO: 84 or SEQ ID NO:85.

14. The recombinant polypeptide of claim 1, wherein the extracellular domain comprises an amino acid sequence having at least 80%, 85%, 90%, 95%, or 100% sequence identity with amino acids 32-123 (Domain I) or amino acids 32-242 (Domains I and II) or amino acids 32-334 (Domains I, II and III) of SEQ ID NO:1.

15. The recombinant polypeptide of claim 1, wherein the recombinant polypeptide comprises an amino acid sequence having at least 80%, 85%, 90%, 95%, or 100% sequence identity with amino acids 32-370 (Domains I, II and III, transmembrane domain and a fragment of the intracellular domain) of SEQ ID NO:1.

16. The recombinant polypeptide of claim 1, wherein the recombinant polypeptide comprises an amino acid sequence having at least 95% sequence identity with amino acids 32 to 123, or 32 to 242, or 32 to 334, or 32 to 342, or 32 to 370 of SEQ ID NO:1.

17. The recombinant polypeptide of claim 1, further comprising a signal peptide.

18. The recombinant polypeptide of claim 17, wherein the signal peptide comprises an amino acid sequence having at least 80%, 85%, 90%, 95%, or 100% sequence identity with the amino acid sequence of SEQ ID NO:58, SEQ ID NO:15, SEQ ID NO:63, SEQ ID NO:64, SEQ ID NO:65, or SEQ ID NO:66.

19. The recombinant polypeptide of claim 1, further comprising a linker between the extracellular domain and the transmembrane domain.

20. The recombinant polypeptide of claim 19, wherein the linker comprises an amino acid sequence having at least 80%, 85%, 90%, 95%, or 100% sequence identity with the amino acid sequence of SEQ ID NO:16, SEQ ID NO:17, SEQ ID NO: 18, SEQ ID NO: 19, SEQ ID NO: 20, SEQ ID NO:21 and SEQ ID NO:22.

21. The recombinant polypeptide of claim 10, wherein the intracellular domain consists of the amino acid sequence of SEQ ID NO:14, SEQ ID NO:80, SEQ ID NO:81, SEQ ID NO: 82, SEQ ID NO:83, SEQ ID NO:84, SEQ ID NO:85; or an amino acid sequence at least 80%, 85%, 90%, or 95% identical thereto.

22. The recombinant polypeptide of claim 1, wherein the recombinant polypeptide comprises the amino acid sequence:
i) selected from the group consisting of SEQ ID NOs: 23-40, or an amino acid sequence at least 80%, 85%, 90%, or 95% identical thereto; or
(ii) selected from the group consisting of SEQ ID NOs: 148-208, or an amino acid sequence at least 80%, 85%, 90%, or 95% identical thereto; or
(iii) selected from the group consisting of SEQ ID NOs: 23-40 or from the group consisting of 148-208, or an amino acid sequence at least 80%, 85%, 90%, or 95% identical thereto.

23. The recombinant polypeptide of claim 1, comprising a heterologous region of at least 4 amino acids in length, wherein the amino acid sequence of the heterologous region is heterologous to IL5Ra, optionally wherein the amino acid sequence of the heterologous region is not present in the amino acid sequence of human IL5Ra of SEQ ID NO:1.

24. A nucleic acid molecule comprising a coding sequence for the recombinant polypeptide of any one of claims 1-23.

25. The nucleic acid molecule of claim 24, wherein the nucleic acid molecule comprises the nucleotide sequence:
(i) selected from the group consisting of SEQ ID NOs: 41-57 and 86, or a nucleotide sequence at least 80%, 85%, 90%, 95% or 100% identical thereto; or
(ii) selected from the group consisting of SEQ ID NOs: 87-147, or a nucleotide sequence at least 80%, 85%, 90%, 95% or 100% identical thereto; or
(iii) selected from the group consisting of SEQ ID NOs: 41-57 and 86, or from the group consisting of SEQ ID NOs: 87-147, or an nucleotide sequence at least 80%, 85%, 90%, 95% or 100% identical thereto.

26. The nucleic acid molecule of claim 24, further comprising a coding sequence for a chimeric antigen receptor (CAR).

27. The nucleic acid molecule of claim 26, wherein the coding sequences for the recombinant polypeptide and the CAR are operably linked to the same promoter such that the two coding sequences are co-transcribed.

28. The nucleic acid molecule of claim 24, wherein the nucleic acid molecule is present in a viral vector, optionally wherein the viral vector is a lentiviral vector or a retroviral vector.

29. A cell comprising the nucleic acid molecule of claim 24.

30. The cell of claim 29, wherein the cell is a human T cell, optionally wherein the human T cell is a human Treg cell.

31. A pharmaceutical composition comprising: (i) the cell of claim 29; and (ii) a pharmaceutically acceptable carrier.

32. A pharmaceutical composition comprising: (i) the nucleic acid molecule of claim 21; and (ii) a pharmaceutically acceptable carrier.

33. A method of treating a patient in need thereof, comprising administering the cell of claim 29 to the patient, optionally wherein the cell is derived from the patient.

34. A method of treating a patient in need thereof, comprising administering the cell of claim 30 to the patient, wherein the cell expresses a CAR specific for an antigen present in a disease the patient is suffering from.

35. The method of claim 34, further comprising administering to the patient an effective amount of an antibody specific for IL5Ra once the patient has been treated, wherein the antibody elicits cytotoxicity against cells expressing the recombinant polypeptide, optionally wherein the antibody is IgG1 or IgG2.

36. A method of making a genetically engineered human cell, comprising providing an isolated human cell, and introducing the nucleic acid molecule of claim 24 into the human cell.

37. The method of claim 36, wherein the human cell is a human T cell.

38. The method of claim 37, wherein the human cell is a human Treg cell.

39. The method of claim 36, further comprising culturing the human cell under conditions for expression of the recombinant polypeptide on the surface of the genetically engineered human cell.

40. A pharmaceutical composition comprising: (i) a plurality of the cells of claim 29, and (ii) a pharmaceutically acceptable carrier.

41. A pharmaceutical composition comprising: (i) a plurality of the genetically engineered human cells produced by the method of claim 36, and (ii) a pharmaceutically acceptable carrier.


SEQUENCE LISTING (208 sequences)

SEQ ID NO: 1 - protein, 420 aa, Homo sapiens
  REGION 1..420: note = IL-5Ra
MIIVAHVLLILLGATEILQADLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSKGRDWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREWFVIVIMATICFILLILSLICKICHLWIKLFPPIPAPKSNIKDLFVTTNYEKAGSSETEIEVICYIEKPGVETLEDSVF

SEQ ID NO: 2 - protein, 92 aa, Homo sapiens
  REGION 1..92: note = IL-5Ra Domain I
PPVNFTIKVTGLAQVLLQWKPNPDQEQRNVNLEYQVKINAPKEDDYETRITESKCVTILHKGFSASVRTILQNDHSLLASSWASAELHAPPG

SEQ ID NO: 3 - protein, 27 aa, Homo sapiens
  REGION 1..27: note = transmembrane domain
FWVLVVVGGVLACYSLLVTVAFIIFWV

SEQ ID NO: 4 - protein, 23 aa, Homo sapiens
  REGION 1..23: note = transmembrane domain
IATGMVGALLLLLVVALGIGLFM

SEQ ID NO: 5 - protein, 23 aa, Homo sapiens
  REGION 1..23: note = transmembrane domain
SIISAVVGILLVVVLGVVFGILI

SEQ ID NO: 6 - protein, 21 aa, Homo sapiens
  REGION 1..21: note = transmembrane domain
VLLCLLLVPLLLSLFVLGLFL

SEQ ID NO: 7 - protein, 21 aa, Homo sapiens
  REGION 1..21: note = transmembrane domain
IIILVGTAVIAMFFWLLLVII

SEQ ID NO: 8 - protein, 21 aa, Homo sapiens
  REGION 1..21: note = transmembrane domain
LIALVTSGALLAVLGITGYFL

SEQ ID NO: 9 - protein, 21 aa, Homo sapiens
  REGION 1..21: note = transmembrane domain
AAVLVLLVIVIISLIVLVVIW

SEQ ID NO: 10 - protein, 21 aa, Homo sapiens
  REGION 1..21: note = transmembrane domain
IYIWAPLAGTCGVLLLSLVIT

SEQ ID NO: 11 - protein, 22 aa, Homo sapiens
  REGION 1..22: note = transmembrane domain
MALIVLGGVAGLLLFIGLGIFF

SEQ ID NO: 12 - protein, 20 aa, Homo sapiens
  REGION 1..20: note = transmembrane domain
FVIVIMATICFILLILSLIC

SEQ ID NO: 13 - protein, 58 aa, Homo sapiens
  REGION 1..58: note = IL-5Ra intracellular domain
KICHLWIKLFPPIPAPKSNIKDLFVTTNYEKAGSSETEIEVICYIEKPGVETLEDSVF

SEQ ID NO: 14 - protein, 8 aa, Homo sapiens
  REGION 1..8: note = IL-5Ra truncated intracellular domain
KICHLWIK

SEQ ID NO: 15 - protein, 22 aa, Homo sapiens
  REGION 1..22: note = signal peptide
MLLLVTSLLLCELPHPAFLLIP

SEQ ID NO: 16 - protein, 12 aa, Homo sapiens
  REGION 1..12: note = IgG4 hinge
ESKYGPPCPPCP

SEQ ID NO: 17 - protein, 119 aa, Homo sapiens
  REGION 1..119: note = IgG4 hinge to CH3
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 18 - protein, 229 aa, synthetic construct
  REGION 1..229: note = artificial hinge
ESKYGPPCPPCPAPEFDGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - protein, 39 aa, Homo sapiens
  REGION 1..39: note = CD28 hinge
IEVMYPPPYLDNEKSNGTIIHVKGKHLCPSPLFPGPSKP

SEQ ID NO: 20 - protein, 45 aa, Homo sapiens
  REGION 1..45: note = CD8 hinge
TTTPAPRPPTPAPTIASQPLSLRPEACRPAAGGAVHTRGLDFACD

SEQ ID NO: 21 - protein, 5 aa, synthetic construct
  REGION 1..5: note = linker
GGGGS

SEQ ID NO: 22 - protein, 7 aa, synthetic construct
  REGION 1..7: note = linker
GGGSGGG

SEQ ID NO: 23 - protein, 362 aa, Homo sapiens
  REGION 1..362: note = IL-5Ra
MIIVAHVLLILLGATEILQADLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSKGRDWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREWFVIVIMATICFILLILSLIC

SEQ ID NO: 24 - protein, 364 aa, synthetic construct
  REGION 1..364: note = IL-5Ra
MLLLVTSLLLCELPHPAFLLIPDLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSKGRDWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREWFVIVIMATICFILLILSLIC

SEQ ID NO: 25 - protein, 372 aa, synthetic construct
  REGION 1..372: note = IL-5Ra
MLLLVTSLLLCELPHPAFLLIPDLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSKGRDWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREWFVIVIMATICFILLILSLICKICHLWIK

SEQ ID NO: 26 - protein, 372 aa, synthetic construct
  REGION 1..372: note = IL-5Ra
MLLLVTSLLLCELPHPAFLLIPDLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSKGRDWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREWFVIVIMATICFILLILSLICKIGHLWIK

SEQ ID NO: 27 - protein, 368 aa, synthetic construct
  REGION 1..368: note = IL-5Ra
MLLLVTSLLLCELPHPAFLLIPDLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSKGRDWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREWFVIVIMATICFILLILSLICKICH

SEQ ID NO: 28 - protein, 152 aa, synthetic construct
  REGION 1..152: note = IL-5Ra
MLLLVTSLLLCELPHPAFLLIPDLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGGGGSGGGFVIVIMATICFILLILSLIC

SEQ ID NO: 29 - protein, 160 aa, synthetic construct
  REGION 1..160: note = IL-5Ra
MLLLVTSLLLCELPHPAFLLIPDLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGGGGGSGGGGSGGGGSFVIVIMATICFILLILSLIC

SEQ ID NO: 30 - protein, 157 aa, synthetic construct
  REGION 1..157: note = IL-5Ra
MLLLVTSLLLCELPHPAFLLIPDLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGESKYGPPCPPCPFVIVIMATICFILLILSLIC

SEQ ID NO: 31 - protein, 155 aa, synthetic construct
  REGION 1..155: note = IL-5Ra
MIIVAHVLLILLGATEILQADLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGESKYGPPCPPCPFVIVIMATICFILLILSLIC

SEQ ID NO: 32 - protein, 156 aa, synthetic construct
  REGION 1..156: note = IL-5Ra
MLLLVTSLLLCELPHPAFLLIPDLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGGGGSGGGFVIVIMATICFILLILSLICKICH

SEQ ID NO: 33 - protein, 164 aa, synthetic construct
MLLLVTSLLLCELPHPAFLLIPDLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGGGGGSGGGGSGGGGSFVIVIMATICFILLILSLICKICH

SEQ ID NO: 34 - protein, 164 aa, synthetic construct; identical to SEQ ID NO: 33
  REGION 1..164: note = IL-5Ra

SEQ ID NO: 35 - protein, 159 aa, synthetic construct
  REGION 1..159: note = IL-5Ra
MIIVAHVLLILLGATEILQADLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGESKYGPPCPPCPFVIVIMATICFILLILSLICKICH

SEQ ID NO: 36 - protein, 268 aa, synthetic construct
  REGION 1..268: note = IL-5Ra
MLLLVTSLLLCELPHPAFLLIPDLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKFVIVIMATICFILLILSLICKICH

SEQ ID NO: 37 - protein, 378 aa, synthetic construct
  REGION 1..378: note = IL-5Ra
MLLLVTSLLLCELPHPAFLLIPDLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGESKYGPPCPPCPAPEFDGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKFVIVIMATICFILLILSLICKICH

SEQ ID NO: 38 - protein, 260 aa, synthetic construct
  REGION 1..260: note = IL-5Ra
MLLLVTSLLLCELPHPAFLLIPDLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGGGGSGGGPPVNFTIKVTGLAQVLLQWKPNPDQEQRNVNLEYQVKINAPKEDDYETRITESKCVTILHKGFSASVRTILQNDHSLLASSWASAELHAPPGESKYGPPCPPCPFVIVIMATICFILLILSLICKICH

SEQ ID NO: 39 - protein, 268 aa, synthetic construct
  REGION 1..268: note = IL-5Ra
MLLLVTSLLLCELPHPAFLLIPDLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGGGGGSGGGGSGGGGSPPVNFTIKVTGLAQVLLQWKPNPDQEQRNVNLEYQVKINAPKEDDYETRITESKCVTILHKGFSASVRTILQNDHSLLASSWASAELHAPPGESKYGPPCPPCPFVIVIMATICFILLILSLICKICH

SEQ ID NO: 40 - protein, 263 aa, synthetic construct
  REGION 1..263: note = IL-5Ra
MLLLVTSLLLCELPHPAFLLIPDLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGGGGGSGGGGSGGGGSPPVNFTIKVTGLAQVLLQWKPNPDQEQRNVNLEYQVKINAPKEDDYETRITESKCVTILHKGFSASVRTILQNDHSLLASSWASAELHAPPGGGGSGGGFVIVIMATICFILLILSLICKICH

SEQ ID NO: 41 - DNA, 1086 bp, synthetic construct
  source 1..1086: mol_type = other DNA
atgataatcgttgcacacgtgttgcttatccttttgggggccacggaaatactccaggcggatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttattttgtcaaagggccgcgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcaatgtgccgggaagcaggactctggagtgaatggtctcagccaatatatgttgggaatgatgagcataagccccttagggaatggttcgtgatagtcattatggcgacaatctgttttattcttttgattctctccctgatttgt

SEQ ID NO: 42 - DNA, 1092 bp, synthetic construct
  source 1..1092: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttattttgtcaaagggccgcgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcaatgtgccgggaagcaggactctggagtgaatggtctcagccaatatatgttgggaatgatgagcataagccccttagggaatggttcgtgatagtcattatggcgacaatctgttttattcttttgattctctccctgatttgt

SEQ ID NO: 43 - DNA, 1116 bp, synthetic construct
  source 1..1116: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttattttgtcaaagggccgcgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcaatgtgccgggaagcaggactctggagtgaatggtctcagccaatatatgttgggaatgatgagcataagccccttagggaatggttcgtgatagtcattatggcgacaatctgttttattcttttgattctctccctgatttgtaaaatctgtcacctttggatcaag

SEQ ID NO: 44 - DNA, 1116 bp, synthetic construct
  source 1..1116: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttattttgtcaaagggccgcgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcaatgtgccgggaagcaggactctggagtgaatggtctcagccaatatatgttgggaatgatgagcataagccccttagggaatggttcgtgatagtcattatggcgacaatctgttttattcttttgattctctccctgatttgtaaaatcggtcacctttggatcaag

SEQ ID NO: 45 - DNA, 456 bp, synthetic construct
  source 1..456: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtggcggtgggagtggtggcggattcgtgatagtcattatggcgacaatctgttttattcttttgattctctccctgatttgt

SEQ ID NO: 46 - DNA, 456 bp, synthetic construct; identical to SEQ ID NO: 45
  source 1..456: mol_type = other DNA

SEQ ID NO: 47 - DNA, 480 bp, synthetic construct
  source 1..480: mol_type = other DNA
atgcttctcctggtgacaagccttctgctctgtgagttaccacacccagcattcctcctgatcccagatctcctgccagatgaaaaaatttccctcctgccgccagtcaacttcactataaaagtaacaggtcttgcccaagttctcctgcaatggaagcctaatcctgaccaggaacagcgcaatgtgaacctggagtaccaggtgaagataaacgctcctaaagaagatgattatgagacgcgaattacggagagcaaatgcgtcacaatcctgcacaagggcttctctgctagtgtccgaactattctccagaacgatcactctcttctcgcatcatcatgggcgtctgctgaacttcacgcaccaccgggtggtggaggcggatcaggtggtggcggaagtggtgggggaggctctttcgtgatagtcattatggcgacaatctgttttattcttttgattctctccctgatttgt

SEQ ID NO: 48 - DNA, 492 bp, synthetic construct
  source 1..492: mol_type = other DNA
atgcttctcctggtgacaagccttctgctctgtgagttaccacacccagcattcctcctgatcccagatctcctgccagatgaaaaaatttccctcctgccgccagtcaacttcactataaaagtaacaggtcttgcccaagttctcctgcaatggaagcctaatcctgaccaggaacagcgcaatgtgaacctggagtaccaggtgaagataaacgctcctaaagaagatgattatgagacgcgaattacggagagcaaatgcgtcacaatcctgcacaagggcttctctgctagtgtccgaactattctccagaacgatcactctcttctcgcatcatcatgggcgtctgctgaacttcacgcaccaccgggtggtggaggcggatcaggtggtggcggaagtggtgggggaggctctttcgtgatagtcattatggcgacaatctgttttattcttttgattctctccctgatttgtaaaatctgtcac

SEQ ID NO: 49 - DNA, 471 bp, synthetic construct
  source 1..471: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtgaaagtaagtacggccccccttgtccgccttgcccgttcgtgatagtcattatggcgacaatctgttttattcttttgattctctccctgatttgt

SEQ ID NO: 50 - DNA, 465 bp, synthetic construct
  source 1..465: mol_type = other DNA
atgataatcgttgcacacgtgttgcttatccttttgggggccacggaaatactccaggcggatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtgagtccaagtacggaccgccttgtccgccttgcccattcgtgatagtcattatggcgacaatctgttttattcttttgattctctccctgatttgt

SEQ ID NO: 51 - DNA, 483 bp, synthetic construct
  source 1..483: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtgaaagtaagtacggccccccttgtccgccttgcccgttcgtgatagtcattatggcgacaatctgttttattcttttgattctctccctgatttgtaaaatctgtcac

SEQ ID NO: 52 - DNA, 477 bp, synthetic construct
  source 1..477: mol_type = other DNA
atgataatcgttgcacacgtgttgcttatccttttgggggccacggaaatactccaggcggatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtgagtccaagtacggaccgccttgtccgccttgcccattcgtgatagtcattatggcgacaatctgttttattcttttgattctctccctgatttgtaaaatctgtcac

SEQ ID NO: 53 - DNA, 804 bp, synthetic construct
  source 1..804: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtgagtccaagtacggaccgccttgtccgccttgcccagggcaacctagggaaccgcaagtctacactcttccaccgtcccaggaggaaatgacgaaaaatcaggtcagtctgacctgccttgtgaaaggattttacccatccgacatagctgttgagtgggaatcaaacggccagcctgaaaataactacaagacgactccacccgtcttggattcagacgggagcttcttcctgtactcccgattgacagtcgataaaagcagatggcaggaaggtaatgtattctcatgcagcgtaatgcacgaggccctccacaaccattatactcagaaaagtctgagcttgagtctgggtaagttcgtgatagtcattatggcgacaatctgttttattcttttgattctctccctgatttgtaaaatctgtcac

SEQ ID NO: 54 - DNA, 1134 bp, synthetic construct
  source 1..1134: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtgagtccaagtacggaccgccttgtccgccttgcccagctccagaatttgatgggggaccgagcgtctttctcttcccccccaagcctaaagataccctcatgatttccaggacaccagaggttacatgtgtcgtggtggatgtttcccaagaagaccctgaggttcagtttaactggtacgttgatggggtagaagtccacaatgccaagacaaagcccagagaggaacagtttcagagtacatatagggtcgtgagtgtacttaccgtgcttcaccaggactggcttaatggtaaagaatataaatgcaaggtttctaataagggcttgccaagtagtatcgaaaaaactatctcaaaagctaaggggcaacctagggaaccgcaagtctacactcttccaccgtcccaggaggaaatgacgaaaaatcaggtcagtctgacctgccttgtgaaaggattttacccatccgacatagctgttgagtgggaatcaaacggccagcctgaaaataactacaagacgactccacccgtcttggattcagacgggagcttcttcctgtactcccgattgacagtcgataaaagcagatggcaggaaggtaatgtattctcatgcagcgtaatgcacgaggccctccacaaccattatactcagaaaagtctgagcttgagtctgggtaagttcgtgatagtcattatggcgacaatctgttttattcttttgattctctccctgatttgtaaaatctgtcac

SEQ ID NO: 55 - DNA, 780 bp, synthetic construct
  source 1..780: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtggcggtgggagtggtggcggaccgccagtcaacttcactataaaagtaacaggtcttgcccaagttctcctgcaatggaagcctaatcctgaccaggaacagcgcaatgtgaacctggagtaccaggtgaagataaacgctcctaaagaagatgattatgagacgcgaattacggagagcaaatgcgtcacaatcctgcacaagggcttctctgctagtgtccgaactattctccagaacgatcactctcttctcgcatcatcatgggcgtctgctgaacttcacgcaccaccgggtgaaagtaagtacggccccccttgtccgccttgcccgttcgtgatagtcattatggcgacaatctgttttattcttttgattctctccctgatttgtaaaatctgtcac

SEQ ID NO: 56 - DNA, 804 bp, synthetic construct
  source 1..804: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtggtggaggcggatcaggtggtggcggaagtggtgggggaggctctccgccagtcaacttcactataaaagtaacaggtcttgcccaagttctcctgcaatggaagcctaatcctgaccaggaacagcgcaatgtgaacctggagtaccaggtgaagataaacgctcctaaagaagatgattatgagacgcgaattacggagagcaaatgcgtcacaatcctgcacaagggcttctctgctagtgtccgaactattctccagaacgatcactctcttctcgcatcatcatgggcgtctgctgaacttcacgcaccaccgggtgaaagtaagtacggccccccttgtccgccttgcccgttcgtgatagtcattatggcgacaatctgttttattcttttgattctctccctgatttgtaaaatctgtcac

SEQ ID NO: 57 - DNA, 789 bp, synthetic construct
  source 1..789: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtggtggaggcggatcaggtggtggcggaagtggtgggggaggctctccgccagtcaacttcactataaaagtaacaggtcttgcccaagttctcctgcaatggaagcctaatcctgaccaggaacagcgcaatgtgaacctggagtaccaggtgaagataaacgctcctaaagaagatgattatgagacgcgaattacggagagcaaatgcgtcacaatcctgcacaagggcttctctgctagtgtccgaactattctccagaacgatcactctcttctcgcatcatcatgggcgtctgctgaacttcacgcaccaccgggtggcggtgggagtggtggcggattcgtgatagtcattatggcgacaatctgttttattcttttgattctctccctgatttgtaaaatctgtcac

SEQ ID NO: 58 - protein, 20 aa, Homo sapiens
  REGION 1..20: note = IL-5Ra signal peptide
MIIVAHVLLILLGATEILQA

SEQ ID NO: 59 - protein, 322 aa, Homo sapiens
  REGION 1..322: note = IL-5Ra extracellular domain
DLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSKGRDWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREW

SEQ ID NO: 60 - protein, 92 aa, Homo sapiens; identical to SEQ ID NO: 2
  REGION 1..92: note = IL-5Ra Domain I (32-123 aa)

SEQ ID NO: 61 - protein, 119 aa, Homo sapiens
  REGION 1..119: note = IL-5Ra Domain II (124-242 aa)
SPGTSIVNLTCTTNTTEDNYSRLRSYQVSLHCTWLVGTDAPEDTQYFLYYRYGSWTEECQEYSKDTLGRNIACWFPRTFILSKGRDWLAVLVNGSSKHSAIRPFDQLFALHAIDQINPP

SEQ ID NO: 62 - protein, 92 aa, Homo sapiens
  REGION 1..92: note = IL-5Ra Domain III (243-334 aa)
LNVTAEIEGTRLSIQWEKPVSAFPIHCFDYEVKIHNTRNGYLQIEKLMTNAFISIIDDLSKYDVQVRAAVSSMCREAGLWSEWSQPIYVGND

SEQ ID NO: 63 - protein, 19 aa, Homo sapiens
  REGION 1..19: note = signal peptide
MQSKVLLAVALWLCVETRA

SEQ ID NO: 64 - protein, 22 aa, Homo sapiens
  REGION 1..22: note = signal peptide
MELAALCRWGLLLALLPPGAAS

SEQ ID NO: 65 - protein, 23 aa, Homo sapiens
  REGION 1..23: note = signal peptide
MGTSHPAFLVLGCLLTGLSLILC

SEQ ID NO: 66 - protein, 22 aa, Homo sapiens
  REGION 1..22: note = signal peptide
MAGSPTCLTLIYILWQLTGSAA

SEQ ID NO: 67 - protein, 103 aa, Homo sapiens
  REGION 1..103: note = IL5Ra Domain I
DLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPG

SEQ ID NO: 68 - protein, 103 aa, Homo sapiens
  REGION 1..103: note = IL5Ra Domain I
DLLPDEKISLLPPVNFTIKVTGLAQVLLQWKPNPDQEQRNVNLEYQVKINAPKEDDYETRITESKCVTALHKGFSASVRTILQNDHSLLASSWASAELHAPPG

SEQ ID NO: 69 - protein, 103 aa, Homo sapiens
  REGION 1..103: note = IL5Ra Domain I
DLLPDEKISLLPPVNFTIKVTGLAQVLLQWKPNPDQEQRNVNLEYQVKINAPKEDDYETRITEAKCVTILHKGFSASVRTILQNDHSLLASSWASAELHAPPG

SEQ ID NO: 70 - protein, 119 aa, Homo sapiens
  REGION 1..119: note = IL5Ra Domain II
SPGTSIVNLTCTTNTTEDNYSRLRSYQVSLHCTWLVGTDAPEDTQYFLYYRYGSWTEECQEYSKDTLGRNIACWFPRTFALSKGRDWLAVLVNGSSKHSAIRPFDQLFALHAIDQINPP

SEQ ID NO: 71 - protein, 119 aa, Homo sapiens
  REGION 1..119: note = IL5Ra Domain II
SPGTSIVNLTCTTNTTEDNYSRLRSYQVSLHCTWLVGTDAPEDTQYFLYYRYGSWTEECQEYSKDTLGRNIACWFPRTFILSAGRDWLAVLVNGSSKHSAIRPFDQLFALHAIDQINPP

SEQ ID NO: 72 - protein, 119 aa, Homo sapiens
  REGION 1..119: note = IL5Ra Domain II
SPGTSIVNLTCTTNTTEDNYSRLRSYQVSLHCTWLVGTDAPEDTQYFLYYRYGSWTEECQEYSKDTLGRNIACWFPRTFILSKGADWLAVLVNGSSKHSAIRPFDQLFALHAIDQINPP

SEQ ID NO: 73 - protein, 100 aa, Homo sapiens
  REGION 1..100: note = IL5Ra Domain III
LNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREW

SEQ ID NO: 74 - protein, 100 aa, Homo sapiens
  REGION 1..100: note = IL5Ra Domain III
LNVTAEIEGTRLSIQWEKPVSAFPIHCFDYEVKIHNTRNGYLQIEKLMTNAFISIIDDLSKYDVQVRAAVSSACREAGLWSEWSQPIYVGNDEHKPLREW

SEQ ID NO: 75 - protein, 100 aa, Homo sapiens
  REGION 1..100: note = IL5Ra Domain III
LNVTAEIEGTRLSIQWEKPVSAFPIHCFDYEVKIHNTRNGYLQIEKLMTNAFISIIDDLSKYDVQVRAAVSSMAREAGLWSEWSQPIYVGNDEHKPLREW

SEQ ID NO: 76 - protein, 100 aa, Homo sapiens
  REGION 1..100: note = IL5Ra Domain III
LNVTAEIEGTRLSIQWEKPVSAFPIHCFDYEVKIHNTRNGYLQIEKLMTNAFISIIDDLSKYDVQVRAAVSSMCAEAGLWSEWSQPIYVGNDEHKPLREW

SEQ ID NO: 77 - protein, 100 aa, Homo sapiens
  REGION 1..100: note = IL5Ra Domain III
LNVTAEIEGTRLSIQWEKPVSAFPIHCFDYEVKIHNTRNGYLQIEKLMTNAFISIIDDLSKYDVQVRAAVSSMCRAAGLWSEWSQPIYVGNDEHKPLREW

SEQ ID NO: 78 - protein, 22 aa, Homo sapiens
VSAVTLAYLIFCLCSLVGILHL

SEQ ID NO: 79 - protein, 21 aa, Homo sapiens
IVAVIFGLLLGAALLLGILVF

SEQ ID NO: 80 - protein, 10 aa, Homo sapiens
LRTVKRANGG

SEQ ID NO: 81 - protein, 9 aa, Homo sapiens
KRRQQKIRK

SEQ ID NO: 82 - protein, 17 aa, Homo sapiens
KQKPRYEIRWRVIESIS

SEQ ID NO: 83 - protein, 12 aa, Homo sapiens
QRALVLRRKRKR

SEQ ID NO: 84 - protein, 8 aa, Homo sapiens
WFLKRERQ

SEQ ID NO: 85 - protein, 16 aa, Homo sapiens
RSRRAQRQRQQRQRDR

SEQ ID NO: 86 - DNA, 1104 bp, synthetic construct
  source 1..1104: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttattttgtcaaagggccgcgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcaatgtgccgggaagcaggactctggagtgaatggtctcagccaatatatgttgggaatgatgagcataagccccttagggaatggttcgtgatagtcattatggcgacaatctgttttattcttttgattctctccctgatttgtaaaatctgtcac

SEQ ID NO: 87 - DNA, 1089 bp, Homo sapiens
  source 1..1089: mol_type = unassigned DNA
atgataatcgttgcacacgtgttgcttatccttttgggggccacggaaatactccaggcggatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttattttgtcaaagggccgcgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcaatgtgccgggaagcaggactctggagtgaatggtctcagccaatatatgttgggaatgatgagcataagccccttagggaatggttcgtgatagtcattatggcgacaatctgttttattcttttgattctctccctgatttgttga

SEQ ID NO: 88 - DNA, 1095 bp, synthetic construct
  source 1..1095: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttattttgtcaaagggccgcgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcaatgtgccgggaagcaggactctggagtgaatggtctcagccaatatatgttgggaatgatgagcataagccccttagggaatggttcgtgatagtcattatggcgacaatctgttttattcttttgattctctccctgatttgttga

SEQ ID NO: 89 - DNA, 1119 bp, synthetic construct
  source 1..1119: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttattttgtcaaagggccgcgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcaatgtgccgggaagcaggactctggagtgaatggtctcagccaatatatgttgggaatgatgagcataagccccttagggaatggttcgtgatagtcattatggcgacaatctgttttattcttttgattctctccctgatttgtaaaatctgtcacctttggatcaagtga

SEQ ID NO: 90 - DNA, 1119 bp, synthetic construct
  source 1..1119: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttattttgtcaaagggccgcgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcaatgtgccgggaagcaggactctggagtgaatggtctcagccaatatatgttgggaatgatgagcataagccccttagggaatggttcgtgatagtcattatggcgacaatctgttttattcttttgattctctccctgatttgtaaaatcggtcacctttggatcaagtga

SEQ ID NO: 91 - DNA, 1107 bp, synthetic construct
  source 1..1107: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttattttgtcaaagggccgcgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcaatgtgccgggaagcaggactctggagtgaatggtctcagccaatatatgttgggaatgatgagcataagccccttagggaatggttcgtgatagtcattatggcgacaatctgttttattcttttgattctctccctgatttgtaaaatctgtcactga

SEQ ID NO: 92 - DNA, 1107 bp, synthetic construct
  source 1..1107: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttattttgtcaaagggccgcgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcaatgtgccgggaagcaggactctggagtgaatggtctcagccaatatatgttgggaatgatgagcataagccccttagggaatggttcgtgatagtcattatggcgacaatctgttttattcttttgattctctccctgatttgtaaaatcggccactga

SEQ ID NO: 93 - DNA, 1113 bp, synthetic construct
  source 1..1113: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttattttgtcaaagggccgcgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcaatgtgccgggaagcaggactctggagtgaatggtctcagccaatatatgttgggaatgatgagcataagccccttagggaatggttcgtgatagtcattatggcgacaatctgttttattcttttgattctctccctgatttgtaaaatctgtcacctttggtga

SEQ ID NO: 94 - DNA, 1113 bp, synthetic construct
  source 1..1113: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttattttgtcaaagggccgcgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcaatgtgccgggaagcaggactctggagtgaatggtctcagccaatatatgttgggaatgatgagcataagccccttagggaatggttcgtgatagtcattatggcgacaatctgttttattcttttgattctctccctgatttgtaaaatcggccacctttggtga

SEQ ID NO: 95 - DNA, 1104 bp, synthetic construct
  source 1..1104: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttattttgtcaaagggccgcgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcaatgtgccgggaagcaggactctggagtgaatggtctcagccaatatatgttgggaatgatgagcataagccccttagggaatggttcgtgatagtcattatggcgacaatctgttttattcttttgattctctccctgatttgtaagcgcaggtga

SEQ ID NO: 96 - DNA, 1113 bp, synthetic construct
  source 1..1113: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttattttgtcaaagggccgcgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcaatgtgccgggaagcaggactctggagtgaatggtctcagccaatatatgttgggaatgatgagcataagccccttagggaatggttcgtgatagtcattatggcgacaatctgttttattcttttgattctctccctgatttgttggtttctcaagagggaatga

SEQ ID NO: 97 - DNA, 1107 bp, synthetic construct
  source 1..1107: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccgccttgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttattttgtcaaagggccgcgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcaatgtgccgggaagcaggactctggagtgaatggtctcagccaatatatgttgggaatgatgagcataagccccttagggaatggttcgtgatagtcattatggcgacaatctgttttattcttttgattctctccctgatttgtaaaatctgtcactga

SEQ ID NO: 98 - DNA, 1107 bp, synthetic construct
  source 1..1107: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgaggctaagtgcgttaccatattgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttattttgtcaaagggccgcgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcaatgtgccgggaagcaggactctggagtgaatggtctcagccaatatatgttgggaatgatgagcataagccccttagggaatggttcgtgatagtcattatggcgacaatctgttttattcttttgattctctccctgatttgtaaaatctgtcactga

SEQ ID NO: 99 - DNA, 1107 bp, synthetic construct
  source 1..1107: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttattttgtcaaagggcgccgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcaatgtgccgggaagcaggactctggagtgaatggtctcagccaatatatgttgggaatgatgagcataagccccttagggaatggttcgtgatagtcattatggcgacaatctgttttattcttttgattctctccctgatttgtaaaatctgtcactga

SEQ ID NO: 100 - DNA, 1107 bp, synthetic construct
  source 1..1107: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgaggctaagtgcgttaccgccttgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttattttgtcaaagggccgcgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcaatgtgccgggaagcaggactctggagtgaatggtctcagccaatatatgttgggaatgatgagcataagccccttagggaatggttcgtgatagtcattatggcgacaatctgttttattcttttgattctctccctgatttgtaaaatctgtcactga

SEQ ID NO: 101 - DNA, 1107 bp, synthetic construct
  source 1..1107: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccgccttgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttgccttgtcaaagggccgcgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcaatgtgccgggaagcaggactctggagtgaatggtctcagccaatatatgttgggaatgatgagcataagccccttagggaatggttcgtgatagtcattatggcgacaatctgttttattcttttgattctctccctgatttgtaaaatctgtcactga

SEQ ID NO: 102 - DNA, 1107 bp, synthetic construct
  source 1..1107: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccgccttgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttattttgtcaaagggcgccgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcaatgtgccgggaagcaggactctggagtgaatggtctcagccaatatatgttgggaatgatgagcataagccccttagggaatggttcgtgatagtcattatggcgacaatctgttttattcttttgattctctccctgatttgtaaaatctgtcactga

SEQ ID NO: 103 - DNA, 1107 bp, synthetic construct
  source 1..1107: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttgccttgtcaaagggcgccgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcaatgtgccgggaagcaggactctggagtgaatggtctcagccaatatatgttgggaatgatgagcataagccccttagggaatggttcgtgatagtcattatggcgacaatctgttttattcttttgattctctccctgatttgtaaaatctgtcactga

SEQ ID NO: 104 - DNA, 1107 bp, synthetic construct
  source 1..1107: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccgccttgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttgccttgtcaaagggcgccgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcaatgtgccgggaagcaggactctggagtgaatggtctcagccaatatatgttgggaatgatgagcataagccccttagggaatggttcgtgatagtcattatggcgacaatctgttttattcttttgattctctccctgatttgtaaaatctgtcactga

SEQ ID NO: 105 - DNA, 1107 bp, synthetic construct
  source 1..1107: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgaggctaagtgcgttaccgccttgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttgccttgtcaaagggcgccgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcaatgtgccgggaagcaggactctggagtgaatggtctcagccaatatatgttgggaatgatgagcataagccccttagggaatggttcgtgatagtcattatggcgacaatctgttttattcttttgattctctccctgatttgtaaaatctgtcactga

SEQ ID NO: 106 - DNA, 1107 bp, synthetic construct
  source 1..1107: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccgccttgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttgctttgtcaaagggcgccgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcaatgtgccgggaagcaggactctggagtgaatggtctcagccaatatatgttgggaatgatgagcataagccccttagggaatggttcgtgatagtcattatggcgacaatctgttttattcttttgattctctccctgatttgtaaaatctgtcactga

SEQ ID NO: 107 - DNA, 1113 bp, synthetic construct
  source 1..1113: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttattttgtcaaagggccgcgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcaatgtgccgggaagcaggactctggagtgaatggtctcagccaatatatgttgggaatgatgagcataagccccttagggaatggttcgtgatagtcattatggcgacaatctgttttattcttttgattctctccctgatttgtaagcgcaggcagcaaaaatga

SEQ ID NO: 108 - DNA, 1269 bp, synthetic construct
  source 1..1269: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccgccttgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttattttgtcaaagggccgcgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcaatgtgccgggaagcaggactctggagtgaatggtctcagccaatatatgttgggaatgatgagcataagccccttagggaatggttcgtgatagtcattatggcgacaatctgttttattcttttgattctctccctgatttgtaaaatctgtcacctttggatcaagctcttcccacctataccagctccgaaatcaaatataaaagacctttttgttacaacgaattacgaaaaagcgggttcaagtgagactgaaattgaggtgatttgttatatcgagaaacctggcgtagaaactctcgaagattctgtcttttga

SEQ ID NO: 109 - DNA, 1152 bp, synthetic construct
  source 1..1152: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttattttgtcaaagggccgcgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcaatgtgccgggaagcaggactctggagtgaatggtctcagccaatatatgttgggaatgatgagcataagccccttagggaatggttcgtgatagtcattatggcgacaatctgttttattcttttgattctctccctgatttgtcaacgagcactggttctgagacgaaagcggaagcgaatgacggaccctacccgacggtga

SEQ ID NO: 110 - DNA, 1131 bp, synthetic construct
  source 1..1131: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttattttgtcaaagggccgcgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcaatgtgccgggaagcaggactctggagtgaatggtctcagccaatatatgttgggaatgatgagcataagccccttagggaatggttcgtgatagtcattatggcgacaatctgttttattcttttgattctctccctgatttgtcaacgagcactggttctgagacgaaagcggaagcgatga

SEQ ID NO: 111 - DNA, 1122 bp, synthetic construct
  source 1..1122: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttattttgtcaaagggccgcgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcaatgtgccgggaagcaggactctggagtgaatggtctcagccaatatatgttgggaatgatgagcataagccccttagggaatggttcgtgatagtcattatggcgacaatctgttttattcttttgattctctccctgatttgtcaacgagcactggttctgagacgaaagtga

SEQ ID NO: 112 - DNA, 1107 bp, synthetic construct
  source 1..1107: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttgccttgtcaaagggccgcgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcaatgtgccgggaagcaggactctggagtgaatggtctcagccaatatatgttgggaatgatgagcataagccccttagggaatggttcgtgatagtcattatggcgacaatctgttttattcttttgattctctccctgatttgtaaaatctgtcactga

SEQ ID NO: 113 - DNA, 1107 bp, synthetic construct
  source 1..1107: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttattttgtcagcaggccgcgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcaatgtgccgggaagcaggactctggagtgaatggtctcagccaatatatgttgggaatgatgagcataagccccttagggaatggttcgtgatagtcattatggcgacaatctgttttattcttttgattctctccctgatttgtaaaatctgtcactga

SEQ ID NO: 114 - DNA, 1107 bp, synthetic construct
  source 1..1107: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttattttgtcaaagggcgctgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcaatgtgccgggaagcaggactctggagtgaatggtctcagccaatatatgttgggaatgatgagcataagccccttagggaatggttcgtgatagtcattatggcgacaatctgttttattcttttgattctctccctgatttgtaaaatctgtcactga

SEQ ID NO: 115 - DNA, 1107 bp, synthetic construct
  source 1..1107: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttattttgtcaaagggccgcgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcagcctgccgggaagcaggactctggagtgaatggtctcagccaatatatgttgggaatgatgagcataagccccttagggaatggttcgtgatagtcattatggcgacaatctgttttattcttttgattctctccctgatttgtaaaatctgtcactga

SEQ ID NO: 116 - DNA, 1107 bp, synthetic construct
  source 1..1107: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttgccttgtcagcaggccgcgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcaatgtgccgggaagcaggactctggagtgaatggtctcagccaatatatgttgggaatgatgagcataagccccttagggaatggttcgtgatagtcattatggcgacaatctgttttattcttttgattctctccctgatttgtaaaatctgtcactga

SEQ ID NO: 117 - DNA, 1107 bp, synthetic construct
  source 1..1107: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttgccttgtcaaagggcgctgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcaatgtgccgggaagcaggactctggagtgaatggtctcagccaatatatgttgggaatgatgagcataagccccttagggaatggttcgtgatagtcattatggcgacaatctgttttattcttttgattctctccctgatttgtaaaatctgtcactga

SEQ ID NO: 118 - DNA, 1107 bp, synthetic construct
  source 1..1107: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttgccttgtcaaagggccgcgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcagcctgccgggaagcaggactctggagtgaatggtctcagccaatatatgttgggaatgatgagcataagccccttagggaatggttcgtgatagtcattatggcgacaatctgttttattcttttgattctctccctgatttgtaaaatctgtcactga

SEQ ID NO: 119 - DNA, 1107 bp, synthetic construct
  source 1..1107: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttattttgtcagcaggcgctgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcaatgtgccgggaagcaggactctggagtgaatggtctcagccaatatatgttgggaatgatgagcataagccccttagggaatggttcgtgatagtcattatggcgacaatctgttttattcttttgattctctccctgatttgtaaaatctgtcactga

SEQ ID NO: 120 - DNA, 1107 bp, synthetic construct
  source 1..1107: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttattttgtcaaagggcgctgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcagcctgccgggaagcaggactctggagtgaatggtctcagccaatatatgttgggaatgatgagcataagccccttagggaatggttcgtgatagtcattatggcgacaatctgttttattcttttgattctctccctgatttgtaaaatctgtcactga

SEQ ID NO: 121 - DNA, 1107 bp, synthetic construct
  source 1..1107: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttgccttgtcagcaggcgctgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcaatgtgccgggaagcaggactctggagtgaatggtctcagccaatatatgttgggaatgatgagcataagccccttagggaatggttcgtgatagtcattatggcgacaatctgttttattcttttgattctctccctgatttgtaaaatctgtcactga

SEQ ID NO: 122 - DNA, 1107 bp, synthetic construct; identical to SEQ ID NO: 121
  source 1..1107: mol_type = other DNA

SEQ ID NO: 123 - DNA, 1107 bp, synthetic construct
  source 1..1107: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttgccttgtcagcaggccgcgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcagcctgccgggaagcaggactctggagtgaatggtctcagccaatatatgttgggaatgatgagcataagccccttagggaatggttcgtgatagtcattatggcgacaatctgttttattcttttgattctctccctgatttgtaaaatctgtcactga

SEQ ID NO: 124 - DNA, 1107 bp, synthetic construct
  source 1..1107: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttgccttgtcaaagggcgctgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcagcctgccgggaagcaggactctggagtgaatggtctcagccaatatatgttgggaatgatgagcataagccccttagggaatggttcgtgatagtcattatggcgacaatctgttttattcttttgattctctccctgatttgtaaaatctgtcactga

SEQ ID NO: 125 - DNA, 1107 bp, synthetic construct
  source 1..1107: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttattttgtcagcaggcgctgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcagcctgccgggaagcaggactctggagtgaatggtctcagccaatatatgttgggaatgatgagcataagccccttagggaatggttcgtgatagtcattatggcgacaatctgttttattcttttgattctctccctgatttgtaaaatctgtcactga

SEQ ID NO: 126 - DNA, 1107 bp, synthetic construct
  source 1..1107: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttgccttgtcagcaggcgctgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcagcctgccgggaagcaggactctggagtgaatggtctcagccaatatatgttgggaatgatgagcataagccccttagggaatggttcgtgatagtcattatggcgacaatctgttttattcttttgattctctccctgatttgtaaaatctgtcactga

SEQ ID NO: 127 - DNA, 1107 bp, synthetic construct
  source 1..1107: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgaggccaagtgcgttaccatattgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttattttgtcaaagggccgcgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcaatgtgccgggaagcaggactctggagtgaatggtctcagccaatatatgttgggaatgatgagcataagccccttagggaatggttcgtgatagtcattatggcgacaatctgttttattcttttgattctctccctgatttgtaaaatctgtcactga

SEQ ID NO: 128 - DNA, 1122 bp, synthetic construct
  source 1..1122: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttattttgtcaaagggccgcgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcaatgtgccgggaagcaggactctggagtgaatggtctcagccaatatatgttgggaatgatgagcataagccccttagggaatggtcaataatcagcgcggttgttgggattcttttggttgttgttctcggagtagtcttcggaatccttataaagcgcaggcagcaaaaatga

SEQ ID NO: 129 - DNA, 1113 bp, synthetic construct
  source 1..1113: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttattttgtcaaagggccgcgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcaatgtgccgggaagcaggactctggagtgaatggtctcagccaatatatgttgggaatgatgagcataagccccttagggaatggtcaataatcagcgcggttgttgggattcttttggttgttgttctcggagtagtcttcggaatccttataaagcgcaggtga

SEQ ID NO: 130 - DNA, 1158 bp, synthetic construct
  source 1..1158: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttattttgtcaaagggccgcgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcaatgtgccgggaagcaggactctggagtgaatggtctcagccaatatatgttgggaatgatgagcataagccccttagggaatgggtgtccgcagtgacgctcgcctatctgatattctgcttgtgcagtcttgttggcatattgcatctccaacgagcactggttctgagacgaaagcggaagcgaatgacggaccctacccgacggtga

SEQ ID NO: 131 - DNA, 1137 bp, synthetic construct
  source 1..1137: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttattttgtcaaagggccgcgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcaatgtgccgggaagcaggactctggagtgaatggtctcagccaatatatgttgggaatgatgagcataagccccttagggaatgggtgtccgcagtgacgctcgcctatctgatattctgcttgtgcagtcttgttggcatattgcatctccaacgagcactggttctgagacgaaagcggaagcgatga

SEQ ID NO: 132 - DNA, 1107 bp, synthetic construct
  source 1..1107: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttattttgtcaaagggccgcgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcaatgtgccgggaagcaggactctggagtgaatggtctcagccaatatatgttgggaatgatgagcataagccccttagggaatgggccgccgtcttggtgctccttgtaatcgtgattatcagtctgattgtcttggtagtaatatggaaacagaagtga

SEQ ID NO: 133 - DNA, 1131 bp, synthetic construct
  source 1..1131: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttattttgtcaaagggccgcgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcaatgtgccgggaagcaggactctggagtgaatggtctcagccaatatatgttgggaatgatgagcataagccccttagggaatgggccgccgtcttggtgctccttgtaatcgtgattatcagtctgattgtcttggtagtaatatggaaacagaagcccaggtacgagataagatggagatga

SEQ ID NO: 134 - DNA, 1149 bp, synthetic construct
  source 1..1149: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttattttgtcaaagggccgcgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcaatgtgccgggaagcaggactctggagtgaatggtctcagccaatatatgttgggaatgatgagcataagccccttagggaatgggccgccgtcttggtgctccttgtaatcgtgattatcagtctgattgtcttggtagtaatatggaaacagaagcccaggtacgagataagatggagagtcatagagtcaataagttga

SEQ ID NO: 135 - DNA, 1119 bp, synthetic construct
  source 1..1119: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttattttgtcaaagggccgcgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcaatgtgccgggaagcaggactctggagtgaatggtctcagccaatatatgttgggaatgatgagcataagccccttagggaatggggtggagaaattgtggcagtgattttcggcctgctgctgggggccgctttactgcttgggatcttagtgtttaggtcccggagatga

SEQ ID NO: 136 - DNA, 1134 bp, synthetic construct
  source 1..1134: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttattttgtcaaagggccgcgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcaatgtgccgggaagcaggactctggagtgaatggtctcagccaatatatgttgggaatgatgagcataagccccttagggaatggggtggagaaattgtggcagtgattttcggcctgctgctgggggccgctttactgcttgggatcttagtgtttaggtcccggagagcgcagaggcagcgatga

SEQ ID NO: 137 - DNA, 1155 bp, synthetic construct
  source 1..1155: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttattttgtcaaagggccgcgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcaatgtgccgggaagcaggactctggagtgaatggtctcagccaatatatgttgggaatgatgagcataagccccttagggaatggggtggagaaattgtggcagtgattttcggcctgctgctgggggccgctttactgcttgggatcttagtgtttaggtcccggagagcgcagaggcagcgacaacagcggcaaagagatcgctga

SEQ ID NO: 138 - DNA, 1122 bp, synthetic construct
  source 1..1122: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttattttgtcagcaggcgctgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcaatgtgccgggaagcaggactctggagtgaatggtctcagccaatatatgttgggaatgatgagcataagccccttagggaatggtcaataatcagcgcggttgttgggattcttttggttgttgttctcggagtagtcttcggaatccttataaagcgcaggcagcaaaaatga

SEQ ID NO: 139 - DNA, 1122 bp, synthetic construct
  source 1..1122: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttattttgtcagcaggcgctgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcagcctgccgggaagcaggactctggagtgaatggtctcagccaatatatgttgggaatgatgagcataagccccttagggaatggtcaataatcagcgcggttgttgggattcttttggttgttgttctcggagtagtcttcggaatccttataaagcgcaggcagcaaaaatga

SEQ ID NO: 140 - DNA, 1269 bp, synthetic construct
  source 1..1269: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttattttgtcaaagggcgctgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcaatgtgccgggaagcaggactctggagtgaatggtctcagccaatatatgttgggaatgatgagcataagccccttagggaatggttcgtgatagtcattatggcgacaatctgttttattcttttgattctctccctgatttgtaaaatctgtcacctttggatcaagctcttcccacctataccagctccgaaatcaaatataaaagacctttttgttacaacgaattacgaaaaagcgggttcaagtgagactgaaattgaggtgatttgttatatcgagaaacctggcgtagaaactctcgaagattctgtcttttga

SEQ ID NO: 141 - DNA, 1269 bp, synthetic construct
  source 1..1269: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttattttgtcagcaggcgctgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcaatgtgccgggaagcaggactctggagtgaatggtctcagccaatatatgttgggaatgatgagcataagccccttagggaatggttcgtgatagtcattatggcgacaatctgttttattcttttgattctctccctgatttgtaaaatctgtcacctttggatcaagctcttcccacctataccagctccgaaatcaaatataaaagacctttttgttacaacgaattacgaaaaagcgggttcaagtgagactgaaattgaggtgatttgttatatcgagaaacctggcgtagaaactctcgaagattctgtcttttga

SEQ ID NO: 142 - DNA, 1269 bp, synthetic construct
  source 1..1269: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttattttgtcagcaggcgctgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcagcctgccgggaagcaggactctggagtgaatggtctcagccaatatatgttgggaatgatgagcataagccccttagggaatggttcgtgatagtcattatggcgacaatctgttttattcttttgattctctccctgatttgtaaaatctgtcacctttggatcaagctcttcccacctataccagctccgaaatcaaatataaaagacctttttgttacaacgaattacgaaaaagcgggttcaagtgagactgaaattgaggtgatttgttatatcgagaaacctggcgtagaaactctcgaagattctgtcttttga

SEQ ID NO: 143 - DNA, 1107 bp, synthetic construct
  source 1..1107: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttattttgtcaaagggccgcgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcaatggcccgggaagcaggactctggagtgaatggtctcagccaatatatgttgggaatgatgagcataagccccttagggaatggttcgtgatagtcattatggcgacaatctgttttattcttttgattctctccctgatttgtaaaatctgtcactga

SEQ ID NO: 144 - DNA, 1107 bp, synthetic construct
  source 1..1107: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttattttgtcaaagggccgcgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcaatgtgcgccgaagcaggactctggagtgaatggtctcagccaatatatgttgggaatgatgagcataagccccttagggaatggttcgtgatagtcattatggcgacaatctgttttattcttttgattctctccctgatttgtaaaatctgtcactga

SEQ ID NO: 145 - DNA, 1107 bp, synthetic construct
  source 1..1107: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttattttgtcaaagggccgcgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcaatgtgccgggccgcaggactctggagtgaatggtctcagccaatatatgttgggaatgatgagcataagccccttagggaatggttcgtgatagtcattatggcgacaatctgttttattcttttgattctctccctgatttgtaaaatctgtcactga

SEQ ID NO: 146 - DNA, 1107 bp, synthetic construct
  source 1..1107: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttattttgtcaaagggcggcgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcaatgtgccgggaagcaggactctggagtgaatggtctcagccaatatatgttgggaatgatgagcataagccccttagggaatggttcgtgatagtcattatggcgacaatctgttttattcttttgattctctccctgatttgtaaaatctgtcactga

SEQ ID NO: 147 - DNA, 1122 bp, synthetic construct
  source 1..1122: mol_type = other DNA
atgcttctcctggtgacaagccttctgctctgtgagttaccacacccagcattcctcctgatcccagatctcctgccagatgaaaaaatttccctcctgccgccagttaactttaccataaaggttacgggacttgctcaagtactcctgcagtggaagccgaaccctgatcaagaacagagaaatgtaaatctcgaataccaggtgaaaataaacgcacccaaggaagatgactatgaaacccgaattactgagagcaagtgcgttaccatattgcataaagggtttagtgctagtgtcagaactatactccagaatgaccacagtttgttggcaagcagttgggccagcgctgagctgcacgcgcctccgggtagccccggtacatccatagttaacctgacttgcaccaccaacacaactgaagacaattattcccgcctgaggtcttatcaagtgtcactgcactgcacttggctggtagggacggatgcgccggaggatacacaatacttcctttattataggtatgggtcctggacagaggaatgtcaagagtactctaaggacacactcgggaggaatatcgcgtgttggtttccccgaacttttattttgtcagcaggccgcgattggctcgcggttttggtaaacggctcatctaaacatagtgctataaggccattcgaccagctgttcgcactccatgctatcgaccagattaaccctccacttaatgtaactgcagagatcgaaggaactagactctccatacagtgggagaaacctgtatccgctttcccgattcactgctttgactatgaggtcaagatccacaacacgcgcaacggttacttgcaaatagagaagctcatgacgaatgcgtttatatctattattgatgacctgtctaaatacgatgttcaggttcgagccgcagttagttcaatgtgccgggaagcaggactctggagtgaatggtctcagccaatatatgttgggaatgatgagcataagccccttagggaatggtcaataatcagcgcggttgttgggattcttttggttgttgttctcggagtagtcttcggaatccttataaagcgcaggcagcaaaaatga

SEQ ID NO: 148 - protein, 362 aa, Homo sapiens; identical to SEQ ID NO: 23

SEQ ID NO: 149 - protein, 364 aa, synthetic construct; identical to SEQ ID NO: 24

SEQ ID NO: 150 - protein, 372 aa, synthetic construct; identical to SEQ ID NO: 25

SEQ ID NO: 151 - protein, 372 aa, synthetic construct; identical to SEQ ID NO: 26

SEQ ID NO: 152 - protein, 368 aa, synthetic construct; identical to SEQ ID NO: 27

SEQ ID NO: 153 - protein, 368 aa, synthetic construct
MLLLVTSLLLCELPHPAFLLIPDLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSKGRDWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREWFVIVIMATICFILLILSLICKIGH

SEQ ID NO: 154 - protein, 370 aa, synthetic construct
MLLLVTSLLLCELPHPAFLLIPDLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSKGRDWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREWFVIVIMATICFILLILSLICKICHLW

SEQ ID NO: 155 - protein, 370 aa, synthetic construct
MLLLVTSLLLCELPHPAFLLIPDLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSKGRDWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREWFVIVIMATICFILLILSLICKIGHLW

SEQ ID NO: 156 - protein, 367 aa, synthetic construct
MLLLVTSLLLCELPHPAFLLIPDLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSKGRDWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREWFVIVIMATICFILLILSLICKRR

SEQ ID NO: 157 - protein, 370 aa, synthetic construct
MLLLVTSLLLCELPHPAFLLIPDLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSKGRDWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREWFVIVIMATICFILLILSLICWFLKRE

SEQ ID NO: 158 - protein, 368 aa, synthetic construct
MLLLVTSLLLCELPHPAFLLIPDLLPDEKISLLPPVNFTIKVTGLAQVLLQWKPNPDQEQRNVNLEYQVKINAPKEDDYETRITESKCVTALHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSKGRDWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREWFVIVIMATICFILLILSLICKICH

SEQ ID NO: 159 - protein, 368 aa, synthetic construct
MLLLVTSLLLCELPHPAFLLIPDLLPDEKISLLPPVNFTIKVTGLAQVLLQWKPNPDQEQRNVNLEYQVKINAPKEDDYETRITEAKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSKGRDWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREWFVIVIMATICFILLILSLICKICH

SEQ ID NO: 160 - protein, 368 aa, synthetic construct
MLLLVTSLLLCELPHPAFLLIPDLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSKGADWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREWFVIVIMATICFILLILSLICKICH

SEQ ID NO: 161 - protein, 368 aa, synthetic construct
MLLLVTSLLLCELPHPAFLLIPDLLPDEKISLLPPVNFTIKVTGLAQVLLQWKPNPDQEQRNVNLEYQVKINAPKEDDYETRITEAKCVTALHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSKGRDWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREWFVIVIMATICFILLILSLICKICH

SEQ ID NO: 162 - protein, 368 aa, synthetic construct
MLLLVTSLLLCELPHPAFLLIPDLLPDEKISLLPPVNFTIKVTGLAQVLLQWKPNPDQEQRNVNLEYQVKINAPKEDDYETRITESKCVTALHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFALSKGRDWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREWFVIVIMATICFILLILSLICKICH

SEQ ID NO: 163 - protein, 368 aa, synthetic construct
MLLLVTSLLLCELPHPAFLLIPDLLPDEKISLLPPVNFTIKVTGLAQVLLQWKPNPDQEQRNVNLEYQVKINAPKEDDYETRITESKCVTALHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSKGADWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREWFVIVIMATICFILLILSLICKICH

SEQ ID NO: 164 - protein, 368 aa, synthetic construct
MLLLVTSLLLCELPHPAFLLIPDLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFALSKGADWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREWFVIVIMATICFILLILSLICKICH

SEQ ID NO: 165 - protein, 368 aa, synthetic construct
MLLLVTSLLLCELPHPAFLLIPDLLPDEKISLLPPVNFTIKVTGLAQVLLQWKPNPDQEQRNVNLEYQVKINAPKEDDYETRITESKCVTALHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFALSKGADWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREWFVIVIMATICFILLILSLICKICH

SEQ ID NO: 166 - protein, 368 aa, synthetic construct
MLLLVTSLLLCELPHPAFLLIPDLLPDEKISLLPPVNFTIKVTGLAQVLLQWKPNPDQEQRNVNLEYQVKINAPKEDDYETRITEAKCVTALHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFALSKGADWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREWFVIVIMATICFILLILSLICKICH

SEQ ID NO: 167 - protein, 368 aa, synthetic construct; identical to SEQ ID NO: 165

SEQ ID NO: 168 - protein, 370 aa, synthetic construct
MLLLVTSLLLCELPHPAFLLIPDLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSKGRDWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREWFVIVIMATICFILLILSLICKRRQQK

SEQ ID NO: 169 - protein, 422 aa, synthetic construct
MLLLVTSLLLCELPHPAFLLIPDLLPDEKISLLPPVNFTIKVTGLAQVLLQWKPNPDQEQRNVNLEYQVKINAPKEDDYETRITESKCVTALHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSKGRDWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREWFVIVIMATICFILLILSLICKICHLWIKLFPPIPAPKSNIKDLFVTTNYEKAGSSETEIEVICYIEKPGVETLEDSVF

SEQ ID NO: 170 - protein, 383 aa, synthetic construct
MLLLVTSLLLCELPHPAFLLIPDLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSKGRDWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREWFVIVIMATICFILLILSLICQRALVLRRKRKRMTDPTRR

SEQ ID NO: 171 - protein, 376 aa, synthetic construct
MLLLVTSLLLCELPHPAFLLIPDLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSKGRDWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREWFVIVIMATICFILLILSLICQRALVLRRKRKR

SEQ ID NO: 172 - protein, 373 aa, synthetic construct
MLLLVTSLLLCELPHPAFLLIPDLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSKGRDWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREWFVIVIMATICFILLILSLICQRALVLRRK

SEQ ID NO: 173 - protein, 368 aa, synthetic construct
MLLLVTSLLLCELPHPAFLLIPDLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFALSKGRDWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREWFVIVIMATICFILLILSLICKICH

SEQ ID NO: 174 - protein, 368 aa, synthetic construct
MLLLVTSLLLCELPHPAFLLIPDLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSAGRDWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREWFVIVIMATICFILLILSLICKICH

SEQ ID NO: 175 - protein, 368 aa, synthetic construct; identical to SEQ ID NO: 160

SEQ ID NO: 176 - protein, 368 aa, synthetic construct
MLLLVTSLLLCELPHPAFLLIPDLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSKGRDWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSACREAGLWSEWSQPIYVGNDEHKPLREWFVIVIMATICFILLILSLICKICH

SEQ ID NO: 177 - protein, 368 aa, synthetic construct
MLLLVTSLLLCELPHPAFLLIPDLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFALSAGRDWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREWFVIVIMATICFILLILSLICKICH

SEQ ID NO: 178 - protein, 368 aa, synthetic construct; identical to SEQ ID NO: 164

SEQ ID NO: 179 - protein, 368 aa, synthetic construct
MLLLVTSLLLCELPHPAFLLIPDLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFALSKGRDWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSACREAGLWSEWSQPIYVGNDEHKPLREWFVIVIMATICFILLILSLICKICH

SEQ ID NO: 180 - protein, 368 aa, synthetic construct
MLLLVTSLLLCELPHPAFLLIPDLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSAGADWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREWFVIVIMATICFILLILSLICKICH

SEQ ID NO: 181 - protein, 368 aa, synthetic construct
MLLLVTSLLLCELPHPAFLLIPDLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSKGADWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSACREAGLWSEWSQPIYVGNDEHKPLREWFVIVIMATICFILLILSLICKICH

SEQ ID NO: 182 - protein, 368 aa, synthetic construct
MLLLVTSLLLCELPHPAFLLIPDLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFALSAGADWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREWFVIVIMATICFILLILSLICKICH

SEQ ID NO: 183 - protein, 368 aa, synthetic construct; identical to SEQ ID NO: 182

SEQ ID NO: 184 - protein, 368 aa, synthetic construct
MLLLVTSLLLCELPHPAFLLIPDLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFALSAGRDWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSACREAGLWSEWSQPIYVGNDEHKPLREWFVIVIMATICFILLILSLICKICH

SEQ ID NO: 185 - protein, 368 aa, synthetic construct
MLLLVTSLLLCELPHPAFLLIPDLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFALSKGADWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSACREAGLWSEWSQPIYVGNDEHKPLREWFVIVIMATICFILLILSLICKICH

SEQ ID NO: 186 - protein, 368 aa, synthetic construct
MLLLVTSLLLCELPHPAFLLIPDLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSAGADWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSACREAGLWSEWSQPIYVGNDEHKPLREWFVIVIMATICFILLILSLICKICH

SEQ ID NO: 187 - protein, 368 aa, synthetic construct
MLLLVTSLLLCELPHPAFLLIPDLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFALSAGADWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSACREAGLWSEWSQPIYVGNDEHKPLREWFVIVIMATICFILLILSLICKICH

SEQ ID NO: 188 - protein, 368 aa, synthetic construct; identical to SEQ ID NO: 159

SEQ ID NO: 189 - protein, 373 aa, synthetic construct
MLLLVTSLLLCELPHPAFLLIPDLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSKGRDWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREWSIISAVVGILLVVVLGVVFGILIKRRQQK

SEQ ID NO: 190 - protein, 370 aa, synthetic construct
MLLLVTSLLLCELPHPAFLLIPDLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSKGRDWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREWSIISAVVGILLVVVLGVVFGILIKRR

SEQ ID NO: 191 - protein, 385 aa, synthetic construct
MLLLVTSLLLCELPHPAFLLIPDLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSKGRDWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREWVSAVTLAYLIFCLCSLVGILHLQRALVLRRKRKRMTDPTRR

SEQ ID NO: 192 - protein, 378 aa, synthetic construct
MLLLVTSLLLCELPHPAFLLIPDLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSKGRDWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREWVSAVTLAYLIFCLCSLVGILHLQRALVLRRKRKR

SEQ ID NO: 193 - protein, 368 aa, synthetic construct
MLLLVTSLLLCELPHPAFLLIPDLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSKGRDWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREWAAVLVLLVIVIISLIVLVVIWKQK

SEQ ID NO: 194 - protein, 376 aa, synthetic construct
MLLLVTSLLLCELPHPAFLLIPDLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSKGRDWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREWAAVLVLLVIVIISLIVLVVIWKQKPRYEIRWR

SEQ ID NO: 195 - protein, 382 aa, synthetic construct
MLLLVTSLLLCELPHPAFLLIPDLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSKGRDWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREWAAVLVLLVIVIISLIVLVVIWKQKPRYEIRWRVIESIS

SEQ ID NO: 196 - protein, 372 aa, synthetic construct
MLLLVTSLLLCELPHPAFLLIPDLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSKGRDWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREWGGEIVAVIFGLLLGAALLLGILVFRSRR

SEQ ID NO: 197 - protein, 377 aa, synthetic construct
MLLLVTSLLLCELPHPAFLLIPDLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSKGRDWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREWGGEIVAVIFGLLLGAALLLGILVFRSRRAQRQR

SEQ ID NO: 198 - protein, 384 aa, synthetic construct
MLLLVTSLLLCELPHPAFLLIPDLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSKGRDWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREWGGEIVAVIFGLLLGAALLLGILVFRSRRAQRQRQQRQRDR

SEQ ID NO: 199 - protein, 373 aa, synthetic construct
MLLLVTSLLLCELPHPAFLLIPDLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSAGADWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREWSIISAVVGILLVVVLGVVFGILIKRRQQK

SEQ ID NO: 200 - protein, 373 aa, synthetic construct
MLLLVTSLLLCELPHPAFLLIPDLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSAGADWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSACREAGLWSEWSQPIYVGNDEHKPLREWSIISAVVGILLVVVLGVVFGILIKRRQQK

SEQ ID NO: 201 - protein, 422 aa, synthetic construct
MLLLVTSLLLCELPHPAFLLIPDLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSKGADWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREWFVIVIMATICFILLILSLICKICHLWIKLFPPIPAPKSNIKDLFVTTNYEKAGSSETEIEVICYIEKPGVETLEDSVF

SEQ ID NO: 202 - protein, 422 aa, synthetic construct
MLLLVTSLLLCELPHPAFLLIPDLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSAGADWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREWFVIVIMATICFILLILSLICKICHLWIKLFPPIPAPKSNIKDLFVTTNYEKAGSSETEIEVICYIEKPGVETLEDSVF

SEQ ID NO: 203 - protein, 422 aa, synthetic construct
MLLLVTSLLLCELPHPAFLLIPDLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSAGADWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSACREAGLWSEWSQPIYVGNDEHKPLREWFVIVIMATICFILLILSLICKICHLWIKLFPPIPAPKSNIKDLFVTTNYEKAGSSETEIEVICYIEKPGVETLEDSVF

SEQ ID NO: 204 - protein, 368 aa, synthetic construct
MLLLVTSLLLCELPHPAFLLIPDLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSKGRDWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMAREAGLWSEWSQPIYVGNDEHKPLREWFVIVIMATICFILLILSLICKICH

SEQ ID NO: 205 - protein, 368 aa, synthetic construct
MLLLVTSLLLCELPHPAFLLIPDLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSKGRDWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCAEAGLWSEWSQPIYVGNDEHKPLREWFVIVIMATICFILLILSLICKICH

SEQ ID NO: 206 - protein, 368 aa, synthetic construct
MLLLVTSLLLCELPHPAFLLIPDLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSKGRDWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCRAAGLWSEWSQPIYVGNDEHKPLREWFVIVIMATICFILLILSLICKICH

SEQ ID NO: 207 - protein, 368 aa, synthetic construct
MLLLVTSLLLCELPHPAFLLIPDLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSKGGDWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREWFVIVIMATICFILLILSLICKICH

SEQ ID NO: 208 - protein, 373 aa, synthetic construct
MLLLVTSLLLCELPHPAFLLIPDLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSAGRDWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREWSIISAVVGILLVVVLGVVFGILIKRRQQK